(19) United States
(12) Patent Application Publication
(10) Pub. No.: US 2023/0162359 A1
(43) Pub. Date: May 25, 2023

(54) DIAGNOSTIC ASSISTANCE METHOD AND DEVICE

(71) Applicant: MEDI WHALE INC., Seoul (KR)

(72) Inventors: Tae Geun CHOI, Seoul (KR); Geun Yeong LEE, Seoul (KR); Young Nam KIM, Seoul (KR)

(21) Appl. No.: 18/089,767

(22) Filed: Dec. 28, 2022

Related U.S. Application Data
(63) Continuation of application No. PCT/KR2020/014951, filed on Oct. 29, 2020.

(30) Foreign Application Priority Data
Jun. 29, 2020 (KR) 10-2020-0079142

(51) Int. Cl. G06T 7/00; A61B 5/00; G06V 10/40; G06V 10/22; G16H 50/20; G16H 30/40

(52) U.S. Cl. CPC G06T 7/0012; A61B 5/4842; G06V 10/40; G06V 10/22; G16H 50/20; G16H 30/40; G06T 2207/20084; G06T 2207/30041; G06T 2207/20081; G06T 2207/30016; G06T 2207/30048

(57) ABSTRACT

An aspect of the present invention relates to a diagnostic assistance device for acquiring diagnostic assistance information by using a neural network model and based on an eye image, the diagnostic assistance device comprising: an eye image acquisition unit for acquiring a target eye image; and a processing unit for acquiring diagnostic assistance information by using a neural network model learned to acquire diagnostic assistance information and based on the target eye image. The neural network model includes first diagnostic assistance neural network model and second diagnostic assistance neural network model for acquiring second diagnostic assistance information. The first diagnostic assistance neural network model includes first common portion for acquiring first feature set and first individual portion for acquiring first diagnostic assistance information, and the second diagnostic assistance neural network model includes first common portion for acquiring first feature set and second individual portion for acquiring second diagnostic assistance information.

### CROSS-REFERENCE TO RELATED APPLICATIONS

This application is a bypass continuation of International PCT Application No. PCT/KR2020/014951, filed on Oct. 29, 2020, which claims priority to Republic of Korea patent application No. 10-2020-0079142, filed on Jun. 29, 2020, which are incorporated by reference herein in their entirety.

### TECHNICAL FIELD

The present invention relates to a diagnosis assistance method and apparatus which uses a neutral network model, and to a diagnosis assistance method and apparatus which obtains a plurality of diagnosis assistance information.

### BACKGROUND ART

The fundus examination is a diagnosis assistance material frequently utilized in ophthalmology since it is able to observe the abnormalities of the retina, optic nerve, and macula and allows the results to be confirmed by relatively simple imaging. In recent years, the fundus examination has been increasingly used because, through the fundus examination, it is able to observe not only eye diseases but also a degree of blood vessel damage caused by chronic diseases such as hypertension and diabetes by a non-invasive method.

Meanwhile, due to the recent rapid development of deep learning technology, the development of diagnostic artificial intelligence has been actively carried out in the field of medical diagnosis, especially the field of image-based diagnosis. Global companies such as Google and IBM have invested heavily in the development of artificial intelligence for analyzing a variety of medical video data, including large-scale data input through collaborations with the medical community. Some companies have succeeded in developing an artificial intelligence diagnostic tool that outputs superior diagnostic results.

However, since it is able to non-invasively observe blood vessels in the body when fundus images are used, there has been a demand for expanding the application of diagnosis using fundus images not only for eye diseases but also for systemic diseases.

### SUMMARY

One object of the present invention is to provide a diagnosis assistance neural network model which obtains a plurality of information.

Objects to be achieved by the present invention are not limited to those mentioned above, and other unmentioned objects should be clearly understood by one of ordinary skill in the art to which the present invention pertains from the present specification and the accompanying drawings.

According to one aspect of the present invention, there is provided a diagnosis assistance apparatus which uses a neural network model including at least one neural network layer and is configured to obtain diagnosis assistance information based on an eye image, the diagnosis assistance apparatus including: an eye image obtaining unit configured to obtain a target eye image which is obtained from eyes of a subject; and a processing unit configured to use a neural network model trained to obtain diagnosis assistance information based on the eye image, and obtain the diagnosis assistance information based on the target eye image, wherein the neural network model includes: first diagnosis assistance neural network model configured to obtain first diagnosis assistance information based on the target eye image; and second diagnosis assistance neural network model configured to obtain second diagnosis assistance information which is different from the first diagnosis assistance information, based on the target eye image, wherein the first diagnosis assistance neural network model includes: first common portion configured to obtain first feature set based on the target eye image; and first individual portion configured to obtain the first diagnosis assistance information based on the first feature set, wherein the second diagnosis assistance neural network model includes: the first common portion configured to obtain the first feature set based on the target eye image; and second individual portion configured to obtain the second diagnosis assistance information based on the first feature set, wherein the first individual portion is trained based on first training data, and the first individual portion is trained based on second training data which is different from the first training data at least in part.

According to another aspect of the present invention, there is provided a method for assisting a diagnosis by using a diagnosis assistance apparatus, the diagnosis assistance apparatus including an eye image obtaining unit configured to obtain an eye image, and a processing unit configured to obtain diagnosis assistance information based on the eye image by using a neural network model, the neural network model including at least one neural network layer and being trained to obtain the diagnosis assistance information based on the eye image, wherein the neural network model includes: first diagnosis assistance neural network model configured to obtain first diagnosis assistance information based on the eye image; and second diagnosis assistance neural network model configured to obtain second diagnosis assistance information based on the eye image, wherein the first diagnosis assistance neural network model includes first common portion and first individual portion, and the second diagnosis assistance neural network model includes the first common portion and second individual portion, wherein the diagnosis assistance method includes: obtaining, by the eye image obtaining unit, a target eye image which is obtained from eyes of a subject; obtaining, by the processing unit, the first feature set based on the target eye image through the first common portion; obtaining, by the processing unit, the first diagnosis assistance information based at least in part on the first feature set through the first individual portion; and obtaining, by the processing unit, the second diagnosis assistance information based at least in part on the first feature set through the second individual portion, wherein the first individual portion is trained based on first training data, and the second individual portion is trained based on second training data which is different from the first training data at least in part.

Technical solutions of the present invention are not limited to those mentioned above, and other unmentioned technical solutions should be clearly understood by one of ordinary skill in the art to which the present invention pertains from the present specification and the accompanying drawing.

According to the present invention, there may be provided a method or an apparatus for assisting a diagnosis based on an eye image.

Advantageous effects of the present invention are not limited to those mentioned above, and other unmentioned advantageous effects should be clearly understood by one of ordinary skill in the art to which the present invention pertains from the present specification and the accompanying drawings.

### BRIEF DESCRIPTION OF DRAWINGS

FIG. 1 illustrates a diagnosis assistance system according to an embodiment of the present invention.

FIG. 2 is a block diagram for describing a training device according to an embodiment of the present invention.

FIG. 3 is a block diagram for describing the training device in more detail according to another embodiment of the present invention.

FIG. 4 is a block diagram for describing a diagnostic device according to an embodiment of the present invention.

FIG. 5 is a view for describing the diagnostic device according to another embodiment of the present invention.

FIG. 6 illustrates a diagnosis assistance system according to an embodiment of the present invention.

FIG. 7 is a block diagram for describing a client device according to an embodiment of the present invention.

FIG. 8 is a view for describing a diagnosis assistance process according to an embodiment of the present invention.

FIG. 9 is a view for describing a configuration of a training unit according to an embodiment of the present invention.

FIG. 10 is a conceptual diagram for describing an image data set according to an embodiment of the present invention.

FIG. 11 is a view for describing image resizing according to an embodiment of the present invention.

FIG. 12 is a view for describing expansion of an image data set according to an embodiment of the present invention.

FIG. 13 is a block diagram for describing a training process of a neural network model according to an embodiment of the present invention.

FIG. 14 is a block diagram for describing a training process of a neural network model according to an embodiment of the present invention.

FIG. 15 is a view for describing a control method of a training device according to an embodiment of the present invention.

FIG. 16 is a view for describing a control method of a training device according to an embodiment of the present invention.

FIG. 17 is a view for describing a control method of a training device according to an embodiment of the present invention.

FIG. 18 is a view for describing a configuration of a diagnostic unit according to an embodiment of the present invention.

FIG. 19 is a view for describing diagnosis target data according to an embodiment of the present invention.

FIG. 20 is a view for describing a diagnostic process according to an embodiment of the present invention.

FIG. 21 is a view for describing a parallel diagnosis assistance system according to some embodiments of the present invention.

FIG. 22 is a view for describing a parallel diagnosis assistance system according to some embodiments of the present invention.

FIG. 23 is a view for describing a configuration of a training device including a plurality of training units according to an embodiment of the present invention.

FIG. 24 is a view for describing a parallel training process according to an embodiment of the present invention.

FIG. 25 is a view for describing the parallel training process according to another embodiment of the present invention.

FIG. 26 is a block diagram for describing a diagnostic unit according to an embodiment of the present invention.

FIG. 27 is a view for describing a diagnosis assistance process according to an embodiment of the present invention.

FIG. 28 is a view for describing a diagnosis assistance system according to an embodiment of the present invention.

FIG. 29 is a view for describing a graphical user interface according to an embodiment of the present invention.

FIG. 30 is a view for describing a graphical user interface according to an embodiment of the present invention.

FIG. 31 is a view for describing a diagnosis assistance neural network model according to an embodiment of the present invention.

FIG. 32 is a view for describing a diagnosis assistance neural network model according to an embodiment of the present invention.

FIG. 33 is a view for describing a diagnosis assistance neural network model according to an embodiment of the present invention.

FIG. 34 is a view for describing a diagnosis assistance neural network model according to an embodiment of the present invention.

FIG. 35 is a view for describing a diagnosis assistance neural network model according to an embodiment of the present invention.

FIG. 36 is a view for describing a diagnosis assistance neural network model according to an embodiment of the present invention.

FIG. 37 is a view for describing a diagnosis assistance neural network model according to an embodiment of the present invention.

FIG. 38 is a view for describing a diagnosis assistance neural network model according to an embodiment of the present invention.

FIG. 39 is a view for describing a diagnosis assistance neural network model according to an embodiment of the present invention.

FIG. 40 is a view for describing a training data set according to an embodiment of the present invention.

FIG. 41 is a view for describing a training method of a diagnosis assistance neural network model according to an embodiment of the present invention.

FIG. 42 is a view for describing a parallel diagnosis assistance system according to some embodiments of the present invention.

FIG. 43 is a view for describing a training method of a diagnosis assistance neural network model according to an embodiment of the present invention.

FIG. 44 is a view for describing a training method of a diagnosis assistance neural network model according to an embodiment of the present invention.

FIG. 45 is a view for describing a training method of a diagnosis assistance neural network model according to an embodiment of the present invention.

FIG. 46 is a view for describing a training method of a diagnosis assistance neural network model according to an embodiment of the present invention.

FIG. 47 is a view for describing a training data set according to an embodiment of the present invention.

FIG. 48 is a view for describing a training method of a diagnosis assistance neural network model according to an embodiment of the present invention.

FIG. 49 is a view for describing a training method of a diagnosis assistance neural network model according to an embodiment of the present invention.

FIG. 50 is a view for describing a training method of a diagnosis assistance neural network model according to an embodiment of the present invention.

FIG. 51 is a view for describing a diagnosis assistance method according to an embodiment of the present invention.

FIG. 52 is a view for describing a diagnosis assistance neural network model according to an embodiment of the present invention.

FIG. 53 is a view for describing a diagnosis assistance neural network model according to an embodiment of the present invention.

FIG. 54 is a view for describing a diagnosis assistance neural network model according to an embodiment of the present invention.

FIG. 55 is a view for describing a diagnosis assistance neural network model according to an embodiment of the present invention.

FIG. 56 is a view for describing a diagnosis assistance neural network model according to an embodiment of the present invention.

FIG. 57 is a view for describing a diagnosis assistance neural network model according to an embodiment of the present invention.

FIG. 58 is a view for describing a training method of a diagnosis assistance neural network model according to an embodiment of the present invention.

FIG. 59 is a view for describing a training method of a diagnosis assistance neural network model according to an embodiment of the present invention.

FIG. 60 is a view for describing a training method of a diagnosis assistance neural network model according to an embodiment of the present invention.

FIG. 61 is a view for describing a diagnosis assistance method according to an embodiment of the present invention.

FIG. 62 is a view for describing a diagnosis assistance neural network model according to an embodiment of the present invention.

FIG. 63 is a view for describing a training method of a diagnosis assistance neural network model according to an embodiment of the present invention.

FIG. 64 is a view for describing a diagnosis assistance method according to an embodiment.

FIG. 65 is a view for describing an image format changing method according to an embodiment of the present invention.

FIG. 66 is a view for describing a method for classifying an eye image according to an embodiment.

FIG. 67 is a view for describing image pre-processing according to an embodiment of the present invention.

FIG. 68 is a view for describing an embodiment for determining whether an eye image satisfies a predetermined criterion.

FIG. 69 is a view for describing a diagnosis assistance neural network model according to an embodiment of the disclosure;

FIG. 70 is a view for describing a diagnosis assistance neural network model according to an embodiment of the present invention.

FIG. 71 is a view for describing a diagnosis assistance method according to an embodiment of the present invention.

FIG. 72 is a view for describing a diagnosis assistance method according to an embodiment of the present invention.

### DETAILED DESCRIPTION

The foregoing objects, features and advantages of the present invention will become more apparent from the following detailed description related to the accompanying drawings. It should be understood, however, that various modifications may be applied to the invention, and the invention may have various embodiments. Hereinafter, specific embodiments, which are illustrated in the drawings, will be described in detail.

In the drawings, the thicknesses of layers and regions are exaggerated for clarity. When it is indicated that an element or layer is “on” or “above” another element or layer, this includes a case in which another layer or element is interposed therebetween as well as a case in which the element or layer is directly above the other element or layer. In principle, like reference numerals designate like elements throughout the specification. In the following description, like reference numerals are used to designate elements which have the same function within the same idea illustrated in the drawings of each embodiment.

When detailed description of known functions or configurations related to the present invention is deemed to unnecessarily blur the gist of the invention, the detailed description thereof will be omitted. Also, numerals (e.g., first, second, etc.) used in the description herein are merely identifiers for distinguishing one element from another element.

In addition, the terms “module” and “unit” used to refer to elements in the following description are given or used in combination only in consideration of ease of writing the specification, and the terms themselves do not have distinct meanings or roles.

A method according to an embodiment may be implemented in the form of a program command that can be executed through various computer means and may be recorded in a computer-readable medium. The computer-readable medium may include program commands, data files, data structures, and the like alone or in combination. The program commands recorded in the medium may be those specially designed and configured for the embodiment or those known to those skilled in the art of computer software and usable. Examples of the computer-readable recording medium include magnetic media such as a hard disk, a floppy disk, and a magnetic tape, optical media such as compact disk-read only memory (CD-ROM), and a digital versatile disk (DVD), magneto-optical media such as a floptical disk, and hardware devices such as a read only memory (ROM), a random access memory (RAM), and a flash memory specially configured to store and execute a program command. Examples of the program command include high-level language codes that may be executed by a computer using an interpreter or the like as well as machine language codes generated by a compiler. The above-mentioned hardware device may be configured to operate as one or more software modules to execute operations according to an embodiment, and vice versa.

### 1. Diagnosis Assistance Using Fundus Image

### 1.1 System and Process for Diagnosis Assistance

### 1.1.1 Purpose and Definition

Hereinafter, a system and method for diagnosis assistance for assisting in determination of the presence of a disease or illness based on a fundus image or the presence of an abnormality which is a basis of the determination will be described. Particularly, a system or method for diagnosis assistance in which a neural network model for diagnosing a disease is constructed using a deep learning technique and detection of the presence of a disease or abnormal findings is assisted using the constructed model will be described.

A machine learning model described in the present specification may be designed based on various machine learning libraries. For example, the machine learning model may refer to various types of models which are designed based on supervised, unsupervised, semi-supervised, or reinforcement learning artificial intelligence (AI) algorithms, such as decision tree, a random forest algorithm, a stochastic gradient descent algorithm, a neural network algorithm, a k-nearest neighbor algorithm, linear regression, logistic regression, support vector machine, K-means, hierarchical cluster analysis (HCA), expectation maximation, principal component analysis (PCA), kernel PCA, locally-linear embedding (LLE), t-distributed stochastic neighbor embedding (t-SNE), Apriori, Eclat, etc.

Hereinafter, the machine learning model will be described as a neural network model for convenience unless particularly described otherwise, but it is obvious that this model does not necessarily mean only a model form that is based on a neural network algorithm, and may be replaced with models that are based on other algorithms with the range of functions and purposes of the invention described in the present specification.

According to an embodiment of the present invention, a system or method for diagnosis assistance in which diagnostic information related to the presence of a disease, findings information used in diagnosis of the presence of a disease, or the like are obtained based on a fundus image and diagnosis is assisted using the obtained information may be provided.

According to an embodiment of the present invention, a system or method for diagnosis assistance in which diagnosis of an eye disease is assisted based on a fundus image may be provided. For example, a system or method for diagnosis assistance in which diagnosis is assisted by obtaining diagnostic information related to the presence of glaucoma, cataract, macular degeneration, retinopathy of prematurity of a testee may be provided.

According to another embodiment of the present invention, a system or method for diagnosis assistance in which diagnosis of a disease other than an eye disease (for example, a systemic disease or a chronic disease) is assisted may be provided. For example, a system or method for diagnosis assistance in which diagnosis is assisted by obtaining diagnostic information on a systemic disease such as hypertension, diabetes, Alzheimer's, cytomegalovirus, stroke, heart disease, and arteriosclerosis may be provided.

According to still another embodiment of the present invention, a system or method for diagnosis assistance for detecting abnormal fundus findings that may be used in diagnosis of an eye disease or other diseases may be provided. For example, a system or method for diagnosis assistance for obtaining findings information such as abnormal color of the entire fundus, opacity of crystalline lens, abnormal cup-to-disc (C/D) ratio, macular abnormalities (e.g., macular hole), an abnormal diameter or course of a blood vessel, an abnormal diameter of the retinal artery, retinal hemorrhage, generation of exudate, and drusen may be provided.

In the specification, diagnosis assistance information may be understood as encompassing diagnostic information according to determination of the presence of a disease, findings information which is a basis of the determination, or the like.

### 1.1.2 Configuration of Diagnosis Assistance System

According to an embodiment of the present invention, a diagnosis assistance system may be provided.

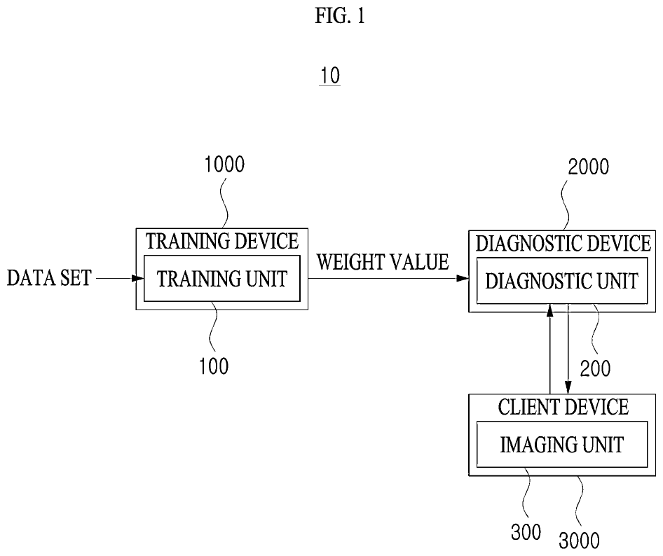

FIG. 1 illustrates a diagnosis assistance system 10 according to an embodiment of the present invention. Referring to FIG. 1, the diagnosis assistance system 10 may include a training device 1000 configured to train a diagnostic model, a diagnostic device 2000 configured to perform diagnosis using the diagnostic model, and a client device 3000 configured to obtain a diagnosis request. The diagnosis assistance system 10 may include a plurality of training devices, a plurality of diagnostic devices, or a plurality of client devices.

The training device 1000 may include a training unit 100. The training unit 100 may perform training of a neural network model. For example, the training unit 100 may obtain a fundus image data set and perform training of a neural network model that detects a disease or abnormal findings from a fundus image.

The diagnostic device 2000 may include a diagnostic unit 200. The diagnostic unit 200 may perform diagnosis of a disease or obtain assistance information used for the diagnosis by using a neural network model. For example, the diagnostic unit 200 may obtain diagnosis assistance information by using a diagnostic model trained by the training unit.

The client device 3000 may include an imaging unit 300. The imaging unit 300 may capture a fundus image. The client device may be an ophthalmic fundus imaging device. Alternatively, the client device 3000 may be a handheld device such as a smartphone or a tablet personal computer (PC).

In the diagnosis assistance system 10 according to the present embodiment, the training device 1000 may obtain a data set and train a neural network model to determine a neural network model to be used in diagnosis assistance, the diagnostic device may obtain diagnosis assistance information according to a diagnosis target image by using the determined neural network model when an information request is obtained from the client device, and the client device may request the diagnostic device for information and obtain diagnosis assistance information transmitted in response to the request.

A diagnosis assistance system according to another embodiment may include a diagnostic device configured to train a diagnostic model and perform diagnosis using the same and may include a client device. A diagnosis assistance system according to still another embodiment may include a diagnostic device configured to train a diagnostic model, obtain a diagnosis request, and perform diagnosis. A diagnosis assistance system according to yet another embodiment may include a training device configured to train a diagnostic model and a diagnostic device configured to obtain a diagnosis request and perform diagnosis.

The diagnosis assistance system disclosed herein is not limited to the above-described embodiments and may be implemented in any form including a training unit configured to train a model, a diagnostic unit configured to obtain diagnosis assistance information according to the trained image, and an imaging unit configured to obtain a diagnosis target image.

Hereinafter, some embodiments of each device constituting the system will be described.

### 1.1.2.1 Training Device

A training device according to an embodiment of the present invention may train a neural network model that assists diagnosis.

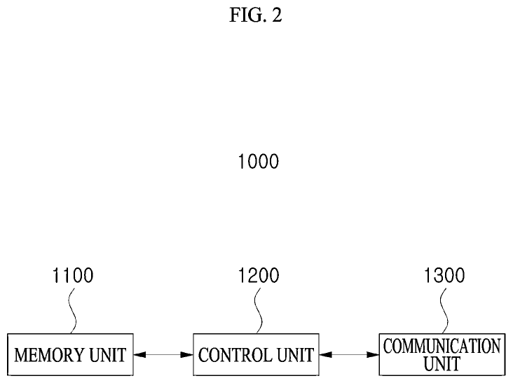

FIG. 2 is a block diagram for describing a training device 1000 according to an embodiment of the present invention. Referring to FIG. 2, the training device 1000 may include a control unit 1200 and a memory unit 1100.

The training device 1000 may include the control unit 1200. The control unit 1200 may control operation of the training device 1000.

The control unit 1200 may include one or more of a central processing unit (CPU), a random access memory (RAM), a graphic processing unit (GPU), one or more microprocessors, and an electronic component capable of processing input data according to predetermined logic.

The control unit 1200 may read a system program and various processing programs stored in the memory unit 1100. For example, the control unit 1200 may develop a data processing process for performing diagnosis assistance which will be described below, a diagnostic process, and the like in a RAM and perform various processes according to a developed program. The control unit 1200 may perform training of a neural network model which will be described below.

The training device 1000 may include the memory unit 1100. The memory unit 1100 may store data required for training and a training model.

The memory unit 1100 may be implemented using a nonvolatile semiconductor memory, a hard disk, a flash memory, a RAM, a ROM, an electrically erasable programmable ROM (EEPROM), or other tangible nonvolatile recording media.

The memory unit 1100 may store various processing programs, parameters for processing programs, result data of such processing, or the like. For example, the memory unit 1100 may store a data processing process program for performing diagnosis assistance which will be described below, a diagnostic process program, parameters for executing each program, data obtained according to execution of such programs (for example, processed data or diagnosis result values), and the like.

The training device 1000 may include a separate training unit (or training module). The training unit may train a neural network model. The training will be described in more detail below in Section “2. Training process.”

The training unit may be included in the above-described control unit 1200. The training unit may be stored in the above-described memory unit 1100. The training unit may be implemented by partial configurations of the above-described control unit 1200 and memory unit 1100. For example, the training unit may be stored in the memory unit 1100 and driven by the control unit 1200.

The training device 1000 may further include a communication unit 1300. The communication unit 1300 may communicate with an external device. For example, the communication unit 1300 may communicate with a diagnostic device, a server device, or a client device which will be described below. The communication unit 1300 may perform wired or wireless communication. The communication unit 1300 may perform bidirectional or unidirectional communication.

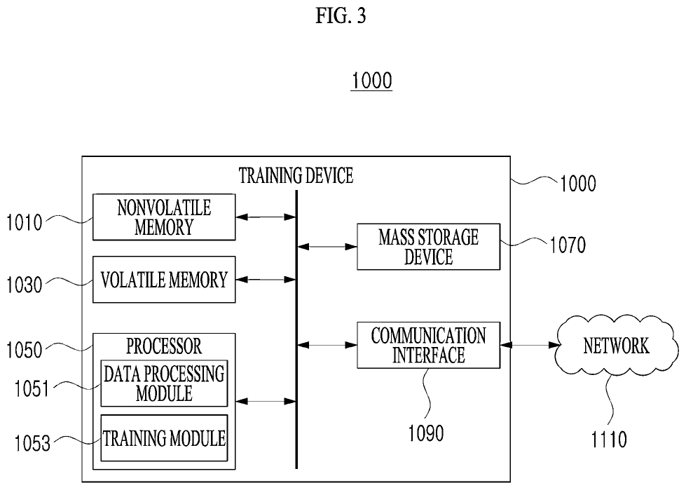

FIG. 3 is a block diagram for describing the training device 1000 in more detail according to another embodiment of the present invention. Referring to FIG. 3, the training device 1000 may include a processor 1050, a volatile memory 1030, a nonvolatile memory 1010, a mass storage device 1070, and a communication interface 1090.

The processor 1050 of the training device 1000 may include a data processing module 1051 and a training module 1053. The processor 1050 may process a data set stored in the mass storage device or nonvolatile memory through the data processing module 1051. The processor 1050 may train a diagnosis assistance neural network model through the training module 1053. The processor 1050 may include a local memory. The communication interface 1090 may be connected to a network 1110.

However, the training device 1000 illustrated in FIG. 3 is merely an example, and the configuration of the training device 1000 according to the present invention is not limited thereto. Particularly, the data processing module or training module may be provided at locations different from those illustrated in FIG. 3.

### 1.1.2.2 Diagnostic Device

A diagnostic device may obtain diagnosis assistance information using a neural network model.

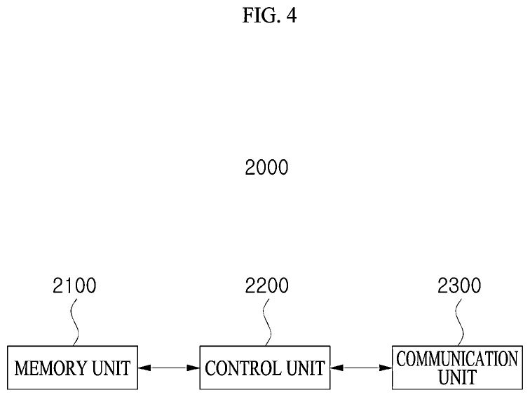

FIG. 4 is a block diagram for describing a diagnostic device 2000 according to an embodiment of the present invention. Referring to FIG. 4, the diagnostic device 2000 may include a control unit 2200 and a memory unit 2100.

The control unit 2200 may generate diagnosis assistance information using a diagnosis assistance neural network model. The control unit 2200 may obtain diagnostic data for diagnosis (for example, fundus data of a testee) and obtain diagnosis assistance information predicted by the diagnostic data using a trained diagnosis assistance neural network model.

The memory unit 2100 may store a trained diagnosis assistance neural network model. The memory unit 2100 may store parameters, variables, and the like of a diagnosis assistance neural network model.

The diagnostic device 2000 may further include a communication unit 2300. The communication unit 2300 may communicate with a training device and/or a client device. For example, the diagnostic device 2000 may be provided in the form of a server that communicates with a client device. This will be described in more detail below.

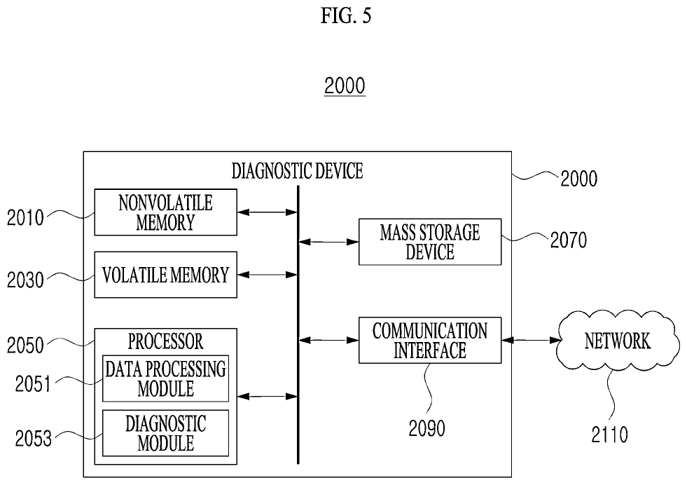

FIG. 5 is a view for describing the diagnostic device 2000 according to another embodiment of the present invention. Referring to FIG. 5, the diagnostic device 2000 according to an embodiment of the present invention may include a processor 2050, a volatile memory 2030, a nonvolatile memory 2010, a mass storage device 2070, and a communication interface 2090.

The processor 2050 of the diagnostic device may include a data processing module 2051 and a diagnostic module 2053. The processor 2050 may process diagnostic data through the data processing module 2051 and obtain diagnosis assistance information according to the diagnostic data through the diagnostic module 2053.

### 1.1.2.3 Server Device

According to an embodiment of the present invention, a diagnosis assistance system may include a server device. The diagnosis assistance system according to an embodiment of the present invention may also include a plurality of server devices.

The server device may store and/or drive a neural network model. The server device may store weights constituting a trained neural network model. The server device may collect or store data used in diagnosis assistance.

The server device may output a result of a diagnosis assistance process using a neural network model to a client device. The server device may obtain feedback from the client device. The server device may operate similar to the above-described diagnostic device.

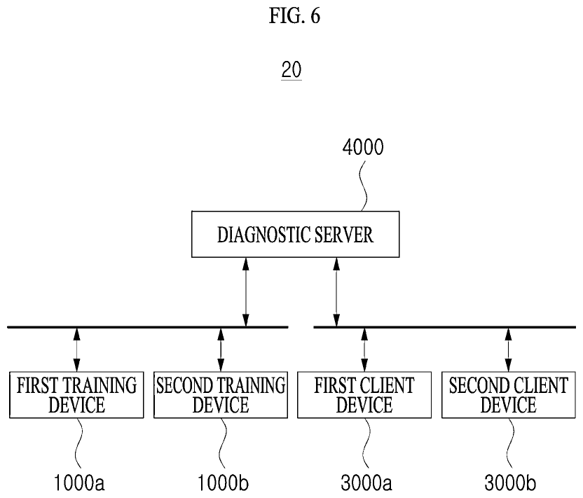

FIG. 6 illustrates a diagnosis assistance system 20 according to an embodiment of the present invention. Referring to FIG. 6, the diagnosis assistance system 20 according to an embodiment of the present invention may include a diagnostic server 4000, a training device, and a client device.

The diagnostic server 4000, i.e., server device, may communicate with a plurality of training devices or a plurality of diagnostic devices. Referring to FIG. 6, the diagnostic server 4000 may communicate with a first training device 1000a and a second training device 1000b. Referring to FIG. 6, the diagnostic server 4000 may communicate with a first client device 3000a and a second client device 3000b.

For example, the diagnostic server 4000 may communicate with the first training device 1000a configured to train a first diagnosis assistance neural network model that obtains a first diagnosis assistance information and the second training device 1000b configured to train a second diagnosis assistance neural network model that obtains a second diagnosis assistance information.

The diagnostic server 4000 may store the first diagnosis assistance neural network model that obtains the first diagnosis assistance information and the second diagnosis assistance neural network model that obtains the second diagnosis assistance information, obtain diagnosis assistance information in response to a request for obtaining diagnosis assistance information from the first client device 3000a or the second client device 3000b, and transmit the obtained diagnosis assistance information to the first client device 3000a or the second client device 3000b.

Alternatively, the diagnostic server 4000 may communicate with the first client device 3000a that requests for the first diagnosis assistance information and the second client device 3000b that requests for the second diagnosis assistance information.

### 1.1.2.4 Client Device

A client device may request a diagnostic device or a server device for diagnosis assistance information. The client device may obtain data required for diagnosis and transmit the obtained data to the diagnostic device.

The client device may include a data obtaining unit. The data obtaining unit may obtain data required for diagnosis assistance. The data obtaining unit may be an imaging unit configured to obtain an image used in a diagnosis assistance model.

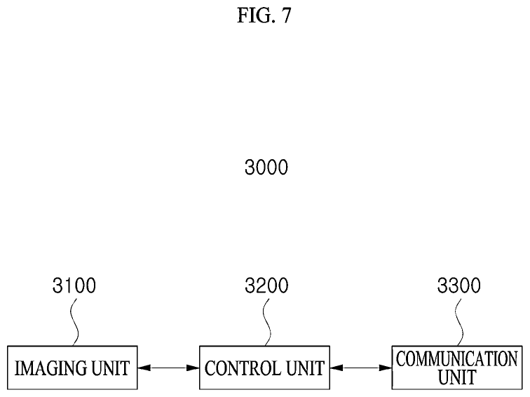

FIG. 7 is a block diagram for describing the client device 3000 according to an embodiment of the present invention. Referring to FIG. 7, the client device 3000 according to an embodiment of the present invention may include an imaging unit 3100, a control unit 3200, and a communication unit 3300.

The imaging unit 3100 may obtain image or video data. The imaging unit 3100 may obtain a fundus image. However, in the client device 3000, the imaging unit 3100 may also be substituted with another form of data obtaining unit.

The communication unit 3300 may communicate with an external device, e.g., a diagnostic device or a server device. The communication unit 3300 may perform wired or wireless communication.

The control unit 3200 may control the imaging unit 3100 to obtain images or data. The control unit 3200 may control the imaging unit 3100 to obtain a fundus image. The control unit 3200 may transmit the obtained fundus image to the diagnostic device. The control unit may transmit an image obtained through the imaging unit 3100 to the server device through the communication unit 3300 and obtain diagnosis assistance information generated based on the obtained image.

Although not illustrated, the client device may further include an output unit. The output unit may include a display configured to output a video or an image or may include a speaker configured to output sound. The output unit may output video or image data obtained by the imaging unit. The output unit may output diagnosis assistance information obtained from the diagnostic device.

Although not illustrated, the client device may further include an input unit. The input unit may obtain a user input. For example, the input unit may obtain a user input that requests for diagnosis assistance information. The input unit may obtain information on a user who evaluates diagnosis assistance information obtained from the diagnostic device.

In addition, although not illustrated, the client device may further include a memory unit. The memory unit may store an image obtained by the imaging unit.

### 1.1.3 Outline of Diagnosis Assistance Process

A diagnosis assistance process may be performed by a diagnosis assistance system or a diagnosis assistance device disclosed herein. The diagnosis assistance process may be taken into consideration by being mainly divided into a training process for training a diagnosis assistance model used in diagnosis assistance and a diagnostic process using the diagnosis assistance model.

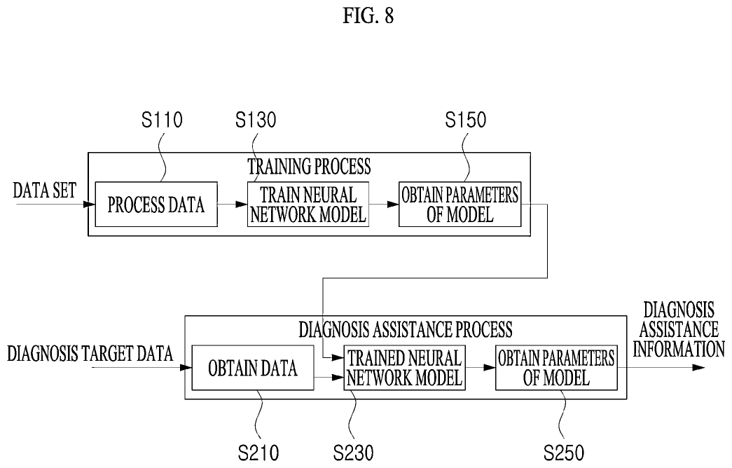

FIG. 8 is a view for describing a diagnosis assistance process according to an embodiment of the present invention. Referring to FIG. 8, the diagnosis assistance process according to an embodiment of the present invention may include a training process including obtaining and processing data (S110), training a neural network model (S130), and obtaining variables of the trained neural network model (S150) and a diagnosis assistance process including obtaining diagnosis target data (S210), using a neural network model trained based on the diagnosis target data (S230), and obtaining diagnosis assistance information using the trained neural network model (S250).

More specifically, the training process may include a data processing process in which input training image data is processed to a state in which the data may be used for model training and a training process in which a model is trained using the processed data. The training process may be performed by the above-described training device.

The diagnostic process may include a data processing process in which input examination target image data is processed to a state in which diagnosis using a neural network model may be performed and a diagnostic process in which diagnosis is performed using the processed data. The diagnostic process may be performed by the above-described diagnostic device or server device.

Hereinafter, each process will be described.

### 1.2 Training Process

According to an embodiment of the present invention, a process for training a neural network model may be provided. As a specific example, a process for training a neural network model that performs or assists diagnosis based on a fundus image may be disclosed.

The training process which will be described below may be performed by the above-described training device.

### 1.2.1 Training Unit

According to an embodiment of the present invention, a training process may be performed by a training unit. The training unit may be provided in the above-described training device.

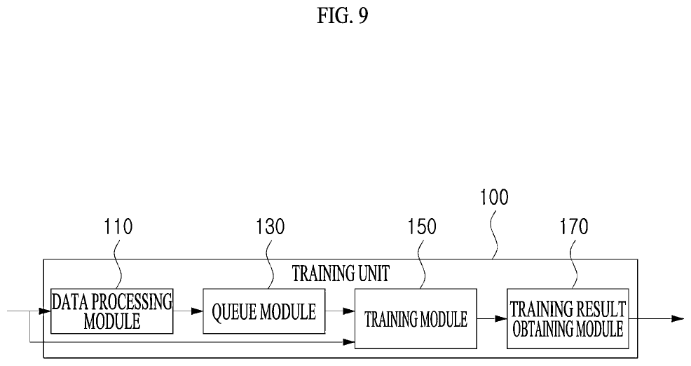

FIG. 9 is a view for describing a configuration of a training unit 100 according to an embodiment of the present invention. Referring to FIG. 9, the training unit 100 may include a data processing module 110, a queue module 130, a training module 150, and a training result obtaining module 170. As will be described below, the modules may perform individual steps of a data processing process and a training process. However, not all of the elements described with reference to FIG. 9 and functions performed by the elements are essential, and some elements may be added or omitted according to a form of training.

### 1.2.2 Data Processing Process

### 1.2.2.1 Obtaining Image Data

According to an embodiment of the present invention, a data set may be obtained. According to an embodiment of the present invention, a data processing module may obtain a data set.

The data set may be an image data set. Specifically, the data set may be a fundus image data set. The fundus image data set may be obtained using a general non-mydriatic fundus camera or the like. A fundus image may be a panorama image. The fundus image may be a red-free image. The fundus image may be an infrared image. The fundus image may be an autofluorescence image. The image data may be obtained in any one format among JPG, PNG, DCM (DICOM), BMP, GIF, and TIFF.

The data set may include a training data set. The data set may include a test data set. The data set may include a validation data set. In other words, the data set may be assigned as at least one of a training data set, a test data set, and a validation data set.

The data set may be determined in consideration of diagnosis assistance information that is desired to be obtained using a neural network model trained through the corresponding data set. For example, when it is desired to train a neural network model that obtains diagnosis assistance information related to cataract, an infrared fundus image data set may be determined as a data set to be obtained. Alternatively, when it is desired to train a neural network model that obtains diagnosis assistance information related to macular degeneration, an obtained data set may be an autofluorescence fundus image data set.

Individual data included in a data set may include a label. There may be a plurality of labels. In other words, individual data included in a data set may be labeled in relation to at least one feature. For example, a data set may be a fundus image data set including a plurality of fundus image data, and each fundus image data may include a label related to diagnostic information (for example, the presence of a specific disease) and/or a label related to findings information (for example, whether a specific site is abnormal) according to the corresponding image.

As another example, a data set may be a fundus image data set, and each fundus image data may include a label related to peripheral information on the corresponding image. For example, each fundus image data may include a label related to peripheral information including left eye/right eye information on whether the corresponding fundus image is an image of the left eye or an image of the right eye, gender information on whether the corresponding fundus image is a fundus image of a female or a fundus image of a male, age information on the age of a testee to which the corresponding fundus image belongs, and the like.

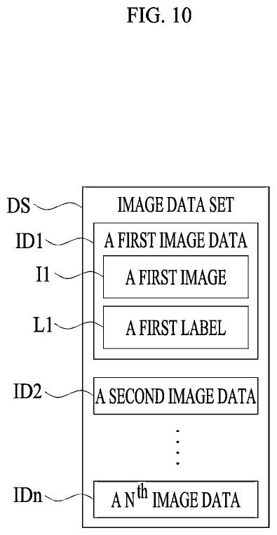

FIG. 10 is a conceptual diagram for describing an image data set DS according to an embodiment of the present invention. Referring to FIG. 10, the image data set DS according to an embodiment of the present invention may include a plurality of image data ID. Each image data ID may include an image I and a label L assigned to the image. Referring to FIG. 10, the image data set DS may include a first image data ID1 and a second image data ID2. The first image data ID1 may include a first image I1 and a first label L1 corresponding to the first image.

Although the case in which a single image data includes a single label has been described above with reference to FIG. 10, a single image data may include a plurality of labels as described above.

### 1.2.2.2 Image Resizing

According to an embodiment of the present invention, the size of an obtained piece of image data may be adjusted. That is, images may be resized. According to an embodiment of the present invention, image resizing may be performed by the data processing module of the above-described training unit.

The size or aspect ratio of an image may be adjusted. Sizes of a plurality of obtained images may be adjusted so that the images have a certain size. Alternatively, the sizes of the images may be adjusted so that the images have a certain aspect ratio. Resizing an image may include applying an image conversion filter to an image.

When the sizes or capacities of obtained individual images are excessively large or small, the size or volume of an image may be adjusted to convert the image to an appropriate size. Alternatively, when the sizes or capacities of individual images vary, the sizes or capacities may be made uniform through resizing.

According to an embodiment, a volume of an image may be adjusted. For example, when a volume of an image exceeds an appropriate range, the image may be reduced through down-sampling. Alternatively, when a volume of an image is below an appropriate range, the image may be enlarged through up-sampling or interpolating.

According to another embodiment, an image may be cut or pixels may be added to an obtained image to adjust the size or aspect ratio of the image. For example, when a portion unnecessary for training is included in an image, a portion of the image may be cropped to remove the unnecessary portion. Alternatively, when a portion of the image is cut away and a set aspect ratio is not met, a column or row may be added to the image to adjust the aspect ratio of the image. In other words, a margin or padding may be added to the image to adjust the aspect ratio.

According to still another embodiment, the volume and the size or aspect ratio of the image may be adjusted together. For example, when a volume of an image is large, the image may be down-sampled to reduce the volume of the image, and an unnecessary portion included in the reduced image may be cropped to convert the image to appropriate image data.

According to another embodiment of the present invention, an orientation of image data may be changed.

As a specific example, when a fundus image data set is used as a data set, the volume or size of each fundus image may be adjusted. Cropping may be performed to remove a margin portion excluding a fundus portion of a fundus image, or padding may be performed to supplement a cut-away portion of a fundus image and adjust an aspect ratio thereof.

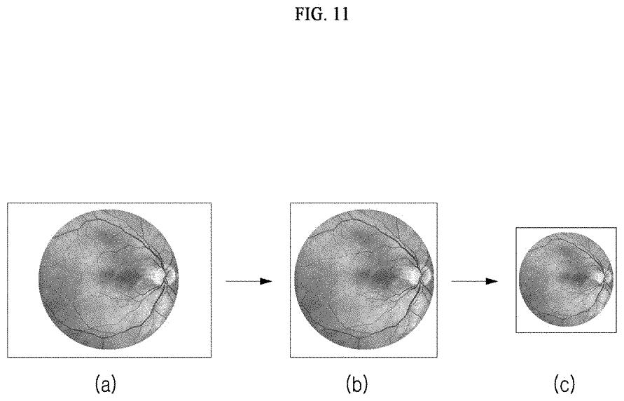

FIG. 11 is a view for describing image resizing according to an embodiment of the present invention. Referring to FIG. 11, an obtained fundus image may be resized by an image resizing process according to an embodiment of the present invention.

Specifically, an original fundus image (a) may be cropped as shown in (b) so that a margin portion unnecessary for obtaining diagnostic information is removed or the size thereof may be reduced as shown in (c) for enhancing the training efficiency.

### 1.2.2.3 Image Pre-Processing

According to an embodiment of the present invention, image pre-processing may be performed. When an input image is used as it is in training, an overfitting phenomenon may occur as a result of a training for unnecessary characteristics, and the training efficiency may also be degraded.

To prevent this, image data may be appropriately pre-processed to serve a purpose of training, thereby improving the efficiency and performance of training. For example, pre-processing of a fundus image may be performed to facilitate detection of abnormal symptoms of an eye disease, or pre-processing of a fundus image may be performed so that changes in retinal vessels or blood flow are emphasized.

Image pre-processing may be performed by the data processing module of the above-described training unit. The data processing module may obtain a resized image and perform pre-processing required for training.

Image pre-processing may be performed on the above-mentioned resized image. However, content of the invention disclosed herein is not limited thereto, and image pre-processing may also be performed without the resizing process. Pre-processing an image may include applying a pre-processing filter to the image.

According to an embodiment, a blur filter may be applied to an image. A Gaussian filter may be applied to an image. A Gaussian blur filter may also be applied to an image. Alternatively, a deblur filter which sharpens an image may be applied to the image.

According to another embodiment, a filter that adjusts or modulates color of an image may be applied. For example, a filter that changes values of some components of RGB values constituting an image or binarizes the image may be applied.

According to still another embodiment, a filter that causes a specific element in an image to be emphasized may be applied to the image. For example, pre-processing that causes a blood vessel element to be emphasized from each image may be performed on fundus image data. In this case, the pre-processing that causes a blood vessel element to be emphasized may include applying one or more filters sequentially or in combination.

According to an embodiment of the present invention, image pre-processing may be performed in consideration of a characteristic of diagnosis assistance information that is desired to be obtained. For example, when it is desired to obtain diagnosis assistance information related to findings such as retinal hemorrhage, drusen, microaneurysms, and exudates, pre-processing that converts an obtained fundus image into a red-free fundus image may be performed.

### 1.2.2.4 Image Augmentation

According to an embodiment of the present invention, an image may be augmented or expanded. Image augmentation may be performed by the data processing module of the above-described training unit.

Augmented images may be used for improving performance of training a neural network model. For example, when an amount of data for training a neural network model is insufficient, existing training image data may be modulated to increase the number of data for training, and modulated (or modified) images may be used together with an original image, thereby increasing the number of training image data. Accordingly, overfitting may be suppressed, layers of a model may be formed deeper, and accuracy of prediction may be improved.

For example, expansion of image data may be performed by reversing the left and right of an image, cutting (cropping) a part of the image, correcting a color value of the image, or adding artificial noise to the image. As a specific example, cutting a part of the image may be performed by cutting a partial region of an element constituting an image or randomly cutting partial regions. In addition, image data may be expanded by reversing the left and right of the image data, reversing the top and bottom of the image data, rotating the image data, resizing the image data to a certain ratio, cropping the image data, padding the image data, adjusting color of the image data, or adjusting brightness of the image data.

For example, the above-described augmentation or expansion of image data may be generally applied to a training data set. However, the augmentation or expansion of image data may also be applied to other data sets, for example, a test data set, i.e., a data set for testing a model on which training using training data and validation using validation data have been completed.

As a specific example, when a fundus image data set is used as a data set, an augmented fundus image data set may be obtained by randomly applying one or more processes of reversing an image, cutting an image, adding noise to an image, and changing color of an image to increase the number of data.

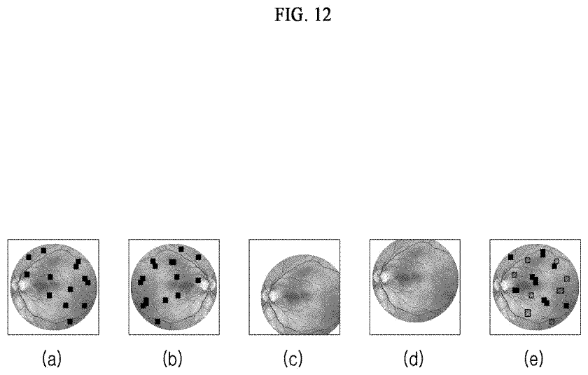

FIG. 12 is a view for describing expansion of an image data set according to an embodiment of the present invention. Referring to FIG. 12, an image according to embodiments of the present invention may be deformed to improve prediction accuracy of a neural network model.

Specifically, referring to FIG. 12, partial regions may be dropped out from an image according to embodiments of the present invention as shown in (a), the left and right of the image may be reversed as shown in (b), a central point of the image may be moved as shown in (c) and (d), and color of partial regions of the image may be modulated as shown in (e).

### 1.2.2.5 Image Serialization

According to an embodiment of the present invention, image data may be serialized. An image may be serialized by the data processing module of the above-described training unit. A serializing module may serialize pre-processed image data and transmit the serialized image data to a queue module.

When image data is used as it is in training, since the image data has an image file format such as JPG, PNG, and DCM, decoding is necessary. However, when training is performed through decoding every time, performance of training a model may be degraded. Accordingly, training may be performed using an serialized image instead of using the image file as it is in training. Therefore, image data may be serialized to improve the performance and speed of training. The image data being serialized may be image data to which one or more steps of the above-described image resizing and image pre-processing are applied or may be image data on which neither the image resizing nor the image pre-processing has been processed.

Each piece of image data included in an image data set may be converted to a string format. Image data may be converted to a binarized data format. Particularly, image data may be converted to a data format suitable for use in training a neural network model. For example, image data may be converted to the TFRecord format for use in training a neural network model using Tensorflow.

As a specific example, when a fundus image set is used as a data set, the obtained fundus image set may be converted to the TFRecord format and used in training a neural network model.

### 1.2.2.6 Queue

A queue may be used for solving a data bottleneck phenomenon. The queue module of the above-described training unit may store image data in a queue and transmit the image data to a training module.

Particularly, when a training process is performed by using a CPU and a GPU together, a bottleneck phenomenon between the CPU and the GPU may be minimized, access to a database may be facilitated, and the memory usage efficiency may be enhanced by using a queue.

A queue may store data used in training a neural network model. The queue may store image data. The image data stored in the queue may be image data on which at least one of the above-described data processing processes (that is, resizing, pre-processing, and augmentation) are processed or may be image data that is unchanged after being obtained.

A queue may store image data, preferably, serialized image data as described above. The queue may store image data and supply the image data to a neural network model. The queue may transfer image data in batch size to a neural network model.

A queue may provide image data. The queue may provide data to a training module which will be described below. As data is extracted from the training module, the number of data accumulated in the queue may be decreased.

When the number of data stored in the queue is decreased to a reference number or lower as training of a neural network model is performed, the queue may request for supplementation of data. The queue may request for supplementation of a specific type of data. When the queue requests the training unit for supplementation of data, the training unit may supplement the queue with data.

A queue may be provided in a system memory of the training device. For example, the queue may be formed in a RAM of a CPU. In this case, the size, i.e., volume, of the queue may be set according to the capacity of the RAM of the CPU. A first-in-first-out (FIFO) queue, a primary queue, or a random queue may be used as the queue.

### 1.2.3 Training Process

According to an embodiment of the present invention, a training process of a neural network model may be disclosed.

According to an embodiment of the present invention, training of a neural network model may be performed by the above-described training device. A training process may be performed by the control unit of the training device. A training process may be performed by the training module of the above-described training unit.

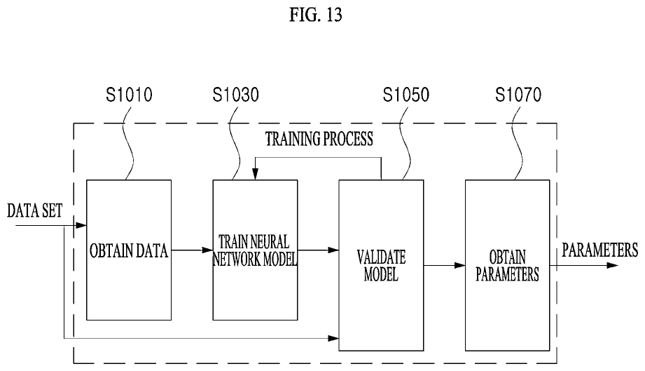

FIG. 13 is a block diagram for describing a training process of a neural network model according to an embodiment of the present invention. Referring to FIG. 13, a training process of a neural network model according to an embodiment of the present invention may be performed by obtaining data (S1010), training a neural network model (S1030), validating the trained model (S1050), and obtaining variables of the trained model (S1070).

Hereinafter, some embodiments of a training process of a neural network model will be described with reference to FIG. 13.

### 1.2.3.1 Data Input

A data set for training a diagnosis assistance neural network model may be obtained.

Obtained data may be an image data set processed by the above-described data processing process. For example, a data set may include fundus image data which is adjusted in size, has a pre-processing filter applied thereto, is augmented and then serialized.

In training a neural network model, a training data set may be obtained and used. In validating the neural network model, a validation data set may be obtained and used. In testing the neural network model, a test data set may be obtained and used. Each data set may include fundus images and labels.

A data set may be obtained from a queue. The data set may be obtained in batches from the queue. For example, when sixty data sets are designated as the size of a batch, sixty data sets may be extracted at a time from the queue. The size of a batch may be limited by the capacity of a RAM of a GPU.

A data set may be randomly obtained from a queue by the training module. Data sets may also be obtained in order of being accumulated in the queue.

The training module may extract a data set by designating a configuration of a data set to be obtained from the queue. For example, the training module may extract fundus image data having a left eye label of a specific subject and fundus image data having a right eye label of the specific subject to be used together in training.

The training module may obtain a data set having a specific label from the queue. For example, the training module may obtain fundus image data in which a diagnostic information label is abnormal label from the queue. The training module may obtain a data set from the queue by designating a ratio between numbers of data according to certain labels. For example, the training module may obtain a fundus image data set from the queue so that the number of fundus image data in which a diagnostic information label is abnormal and the number of fundus image data in which the diagnostic information label is normal has a 1:1 ratio.

### 1.2.3.2 Model Design

A neural network model may be a diagnosis assistance model that outputs diagnosis assistance information based on image data. A structure of a diagnosis assistance neural network model for obtaining diagnosis assistance information may have a predetermined form. The neural network model may include a plurality of layers.

A neural network model may be implemented in the form of a classifier that generates diagnosis assistance information. The classifier may perform binary classification or multiclass classification. For example, a neural network model may be a binary classification model that classifies input data as a normal or abnormal class in relation to target diagnosis assistance information such as a specific disease or abnormal symptoms. Alternatively, a neural network model may be a multiclass classification model that classifies input data into a plurality of classes in relation to a specific characteristic (for example, a degree of disease progression). Alternatively, a neural network model may be implemented as a regression model that outputs specific values related to a specific disease.

A neural network model may include a convolutional neural network (CNN). As a CNN structure, at least one of AlexNet, LENET, NIN, VGGNet, ResNet, WideResnet, GoogleNet, FractaNet, DenseNet, FitNet, RitResNet, HighwayNet, MobileNet, and DeeplySupervisedNet may be used. The neural network model may be implemented using a plurality of CNN structures.

For example, a neural network model may be implemented to include a plurality of VGGNet blocks. As a more specific example, a neural network model may be provided by coupling between a first structure in which a 3×3 CNN layer having 64 filters, a batch normalization (BN) layer, and a ReLu layer are sequentially coupled and a second block in which a 3×3 CNN layer having 128 filters, a ReLu layer, and a BN layer are sequentially coupled.

A neural network model may include a max pooling layer subsequent to each CNN block and include a global average pooling (GAP) layer, a fully connected (FC) layer, and an activation layer (for example, sigmoid, softmax, and the like) at an end.

### 1.2.3.3 Model Training

A neural network model may be trained using a training data set.

A neural network model may be trained using a labeled data set. However, a training process of a diagnosis assistance neural network model described herein is not limited thereto, and a neural network model may also be trained in an unsupervised form using unlabeled data.

Training of a neural network model may be performed by obtaining a result value using a neural network model to which arbitrary weights are assigned based on training image data, comparing the obtained result value with a label value of the training data, and performing backpropagation according to an error therebetween to optimize the weights. Also, training of a neural network model may be affected by a result of validating the model, a result of testing the model, and/or feedback on the model received from the diagnosis step.

The above-described training of a neural network model may be performed using Tensorflow. However, the present invention is not limited thereto, and a framework such as Theano, Keras, Caffe, Torch, and Microsoft Cognitive Toolkit (CNTK) may also be used in training a neural network model.

### 1.2.3.4 Model Validation

A neural network model may be validated using a validation data set. Validation of a neural network model may be performed by obtaining a result value related to a validation data set from a neural network model which has been trained and comparing the result value with a label of the validation data set. The validation may be performed by measuring accuracy of the result value. Parameters of a neural network model (for example, weights and/or bias) or hyperparameters (for example, learning rate) of the neural network model may be adjusted according to a validation result.

For example, the training device according to an embodiment of the present invention may train a neural network model that predicts diagnosis assistance information based on a fundus image and compare diagnosis assistance information on a validated fundus image of the trained model with a validation label corresponding to the validated fundus image to perform validation of the diagnosis assistance neural network model.

In validation of a neural network model, an external data set, that is, a data set having a distinguished factor not included in a training data set, may be used. For example, the external data set may be a data set in which factors such as race, environment, age, and gender are distinguished from the training data set.

### 1.2.3.5 Model Test

A neural network model may be tested using a test data set.

Although not illustrated in FIG. 13, according to the training process according to an embodiment of the present invention, a neural network model may be tested using a test data set which is differentiated from a training data set and a validation data set. Parameters of a neural network model (for example, weights and/or bias) or hyperparameters (for example, learning rate) of the neural network model may be adjusted according to a test result.

For example, the training device according to an embodiment of the present invention may obtain a result value which has test fundus image data, which has not been used in the training and validation, as input from the neural network model which has been trained to predict diagnosis assistance information based on a fundus image and may perform testing of the diagnosis assistance neural network model which has been trained and validated.

In testing of the neural network model, an external data set, that is, a data set having a factor distinguished from the training data set and/or validation data set, may be used.

### 1.2.3.6 Output of Result

As a result of training a neural network model, optimized parameter values of the model may be obtained. As training of the model using a test data set as described above is repeatedly performed, more appropriate parameter (variable) values may be obtained. When the training is sufficiently performed, optimized values of weights and/or bias may be obtained.

According to an embodiment of the present invention, a trained neural network model and/or parameters or variables of the trained neural network model may be stored in the training device and/or diagnostic device (or server). The trained neural network model may be used in predicting diagnosis assistance information by the diagnostic device and/or client device. Also, the parameters or variables of the trained neural network model may be updated by feedback obtained from the diagnostic device or client device.

### 1.2.3.7 Model Ensemble

According to an embodiment of the present invention, in a process of training a single diagnosis assistance neural network model, a plurality of sub-models may be simultaneously trained. The plurality of sub-models may have different layer structures.

In this case, the diagnosis assistance neural network model according to an embodiment of the present invention may be implemented by combining a plurality of sub-neural network models. In other words, training of a neural network model may be performed using an ensemble technique in which a plurality of sub-neural network models are combined.

When a diagnosis assistance neural network model is configured by forming an ensemble, since prediction may be performed by synthesizing results predicted from various forms of sub-neural network models, accuracy of result prediction may be further improved.

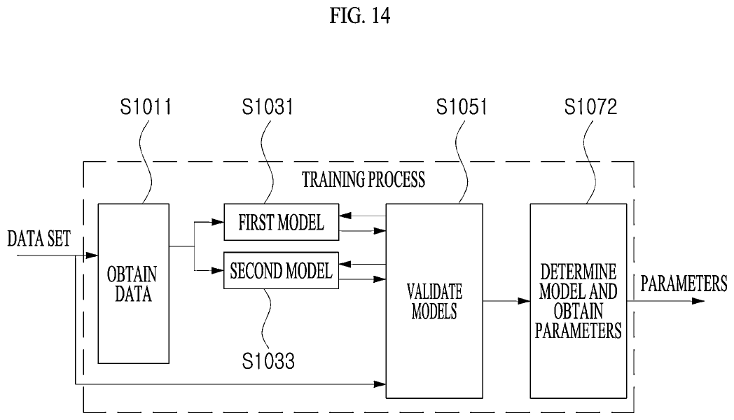

FIG. 14 is a block diagram for describing a training process of a neural network model according to an embodiment of the present invention. Referring to FIG. 14, the training process of a neural network model according to an embodiment of the present invention may include obtaining a data set (S1011), training a first model (that is, first neural network model) and a second model (that is, second neural network model) using the obtained data (S1031, S1033), validating the trained first neural network model and second neural network model (S1051), and determining a final neural network model and obtaining parameters or variables thereof (S1072).

Hereinafter, some embodiments of the training process of a neural network model will be described with reference to FIG. 14.

According to an embodiment of the present invention, a plurality of sub-neural network models may obtain the same training data set and individually generate output values. In this case, an ensemble of the plurality of sub-neural network models may be determined as a final neural network model, and parameter values related to each of the plurality of sub-neural network models may be obtained as training results. An output value of the final neural network model may be set to an average value of the output values by the sub-neural network models. Alternatively, in consideration of accuracy obtained as a result of validating each of the sub-neural network models, the output value of the final neural network model may be set to a weighted average value of the output values of the sub-neural network models.

As a more specific example, when a neural network model includes a first sub-neural network model and a second sub-neural network model, optimized parameter values of the first sub-neural network model and optimized parameter values of the second sub-neural network model may be obtained by machine learning. In this case, an average value of output values (for example, probability values related to specific diagnosis assistance information) obtained from the first sub-neural network model and second sub-neural network model may be determined as an output value of the final neural network model.

According to another embodiment of the present invention, accuracy of individual sub-neural network models may be evaluated based on output values by each of the plurality of sub-neural network models. In this case, any one of the plurality of sub-neural network models may be selected based on the accuracy and determined as the final neural network model. A structure of the determined sub-neural network model and parameter values of the determined sub-neural network model obtained as a result of training may be stored.

As a more specific example, when a neural network model includes a first sub-neural network model and a second sub-neural network model, accuracies of the first sub-neural network model and second sub-neural network model may be obtained, and a more accurate sub-neural network model may be determined as the final neural network model.

According to still another embodiment of the present invention, one or more sub-neural network models among a plurality of neural network models may be combined, ensembles of the one or more combined sub-neural network models may be formed, and each ensemble may be evaluated, wherein a combination of sub-neural network models which forms the most accurate ensemble among the plurality of ensembles may be determined as a final neural network model. In this case, an ensemble may be formed for all possible cases of selecting one or more of the plurality of sub-neural network models, and a combination of sub-neural network models which is evaluated to be the most accurate may be determined as a final neural network model.

As a more specific example, when a neural network model includes a first sub-neural network model and a second sub-neural network model, accuracy of the first sub-neural network model, accuracy of the second sub-neural network model, and accuracy of an ensemble of the first and second sub-neural network models may be compared, and a sub-neural network model combination of the most accurate case may be determined as a final neural network model.

### 1.2.4 Embodiment 1—Control Method of Training Device

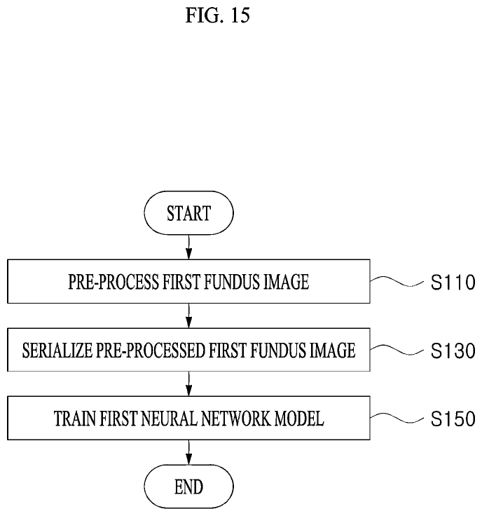

FIG. 15 is a view for describing a control method of a training device according to an embodiment of the present invention.

Referring to FIG. 15, the control method of a training device according to an embodiment of the present invention may include pre-processing a first fundus image (S110), serializing the pre-processed first fundus image (S130), and training a first neural network model (S150).

The control method of a training device according to an embodiment of the present invention may be a control method of a training device included in a system including a training device configured to obtain a first training data set including a plurality of fundus images, process the fundus images included in the first training data set, and train a first neural network model using the first training data set and a diagnostic device configured to obtain a target fundus image for obtaining diagnosis assistance information and obtain the diagnosis assistance information based on the target fundus image by using the trained first neural network model.

The pre-processing of the first fundus image (S110) may further include pre-processing the first fundus image so that the first fundus image included in the first training data set is converted to a format suitable for training the first neural network model.

The control method of the training device according to an embodiment of the present invention may include the serializing of the pre-processed first fundus image (S130). The first fundus image may be serialized to a format that facilitates training of the neural network model.

In this case, the training of the first neural network model (S150) may further include training the first neural network model that classifies the target fundus image as a first label or a second label by using the serialized first fundus image.

The training device may obtain a second training data set which includes the plurality of fundus images and at least partially differs from the first training data set and may train a second neural network model using the second training data set.

According to an embodiment of the present invention, the control method of the training device may further include pre-processing a second fundus image so that the second fundus image included in the second data training set is suitable for training the second neural network model, serializing the pre-processed second fundus image, and training the second neural network model that classifies the target fundus image as a third label or a fourth label by using the serialized second fundus image.

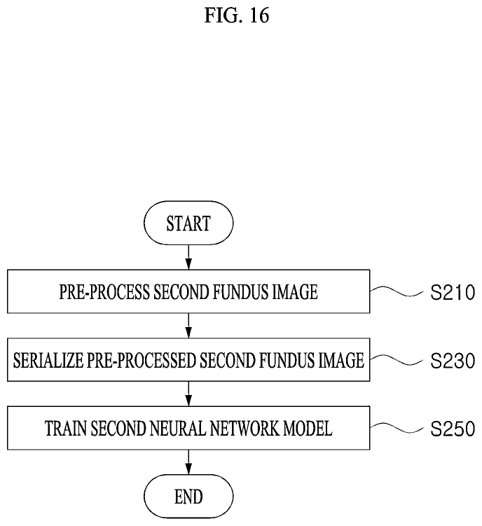

FIG. 16 is a view for describing a control method of a training device according to an embodiment of the present invention. Referring to FIG. 16, the control method of a training device according to an embodiment of the present invention may include pre-processing a second fundus image (S210), serializing the pre-processed second fundus image (S230), and training a second neural network model (S250).

Although, for convenience of description, it has been depicted in FIG. 16 that the pre-processing of the second fundus image, the serializing of the second fundus image, and the training using the second fundus image may be performed subsequent to the pre-processing of the first fundus image, the serializing of the first fundus image, and the training using the first fundus image, content of the invention is not limited thereto.

The pre-processing of the second fundus image included in the second training data set, the serializing of the second fundus image, and the training using the second fundus image may be performed independently of the above-described pre-processing of the first fundus image, serializing of the first fundus image, and training using the first fundus image. The pre-processing of the second fundus image included in the second training data set, the serializing of the second fundus image, and the training using the second fundus image may be performed in parallel with the above-described pre-processing of the first fundus image, serializing of the first fundus image, and training using the first fundus image. In other words, the pre-processing of the second fundus image included in the second training data set, the serializing of the second fundus image, and the training using the second fundus image are not necessarily performed subsequent or prior to the above-described pre-processing of the first fundus image, serializing of the first fundus image, and training using the first fundus image. The process related to the first fundus image and the process related to the second fundus image may be performed without dependence on each other.

First pre-processing performed in relation to the fundus image included in the first training data set may be distinguished from second pre-processing performed in relation to the fundus image included in the second training data set. For example, the first pre-processing may be pre-processing for emphasizing a blood vessel, and the second pre-processing may be pre-processing for modulating color. Each pre-processing may be determined in consideration of diagnosis assistance information desired to be obtained through each neural network model.

The control method of the training device according to an embodiment of the present invention may further include validating the first neural network model by evaluating accuracy of the trained first neural network model by using a first validation data set that is at least partially distinguished from the first training data set and validating the second neural network model by evaluating accuracy of the trained second neural network model by using a second validation data set that is at least partially distinguished from the second training data set. In this case, validation of the first neural network model and validation of the second neural network model may be performed independently of each other.

Serialized first fundus images may be sequentially stored in a first queue, and a predetermined unit volume of the serialized fundus images stored in the first queue may be used each time in training the first neural network model. Serialized second fundus images may be sequentially stored in a second queue distinguished from the first queue, and a predetermined unit volume of the serialized fundus images stored in the second queue may be used each time in training the second neural network model.

The first neural network model may include a first sub-neural network model and a second sub-neural network model. In this case, classifying a target fundus image as the first label or the second label may be performed by simultaneously taking into consideration a first predicted value predicted by the first sub-neural network model and a second predicted value predicted by the second sub-neural network model.

The second neural network model may include a third sub-neural network model and a fourth sub-neural network model. In this case, classifying a target fundus image as the third label or the fourth label may be performed by simultaneously taking into consideration a third predicted value predicted by the third sub-neural network model and a fourth predicted value predicted by the fourth sub-neural network model.

The first training data set may include at least some of fundus images labeled with the first label, and the second training data set may include at least some of fundus images labeled with the third label. In this case, the fundus images labeled with the first label may be the same as at least some of the fundus images labeled with the third label.

The first label may be a normal label indicating that a subject corresponding to the target fundus image is normal in relation to a first finding, and the second label may be an abnormal label indicating that the subject is abnormal in relation to a second finding.

The pre-processing of the first fundus image may include cropping the first fundus image so that a reference aspect ratio is satisfied and changing the size of the first fundus image.

The pre-processing of the first fundus image may further include, by a processing unit, applying a blood vessel emphasizing filter to the fundus image so that a blood vessel included in the first fundus image is emphasized.

Serialized first fundus images may be sequentially stored in a queue, and a predetermined number of the serialized first fundus images stored in the queue may be used each time in training the first neural network model. When the capacity of the serialized first fundus images which have not been used in the training of the first neural network model is reduced to a reference amount or lower, the queue may request for supplementation of the serialized first fundus images.

The first finding may be any one of a finding of retinal hemorrhage, a finding of generation of retinal exudates, a finding of opacity of crystalline lens, and a finding of diabetic retinopathy.

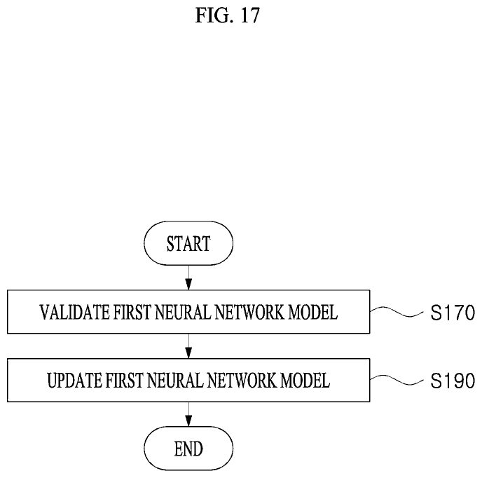

FIG. 17 is a view for describing a control method of a training device according to an embodiment of the present invention.

Referring to FIG. 17, the control method of the training device according to an embodiment of the present invention may further include validating the first neural network model (S170) and updating the first neural network model (S190).

The validating of the first neural network model (S170) may further include validating the first neural network model by evaluating accuracy of the trained first neural network model by using the first validation data set that is at least partially distinguished from the first training data set.

The updating of the first neural network model (S190) may further include updating the first neural network model by reflecting a validation result obtained from the validating of the first neural network model (S170).

Meanwhile, the first neural network model may include a first sub-neural network model and a second sub-neural network model. In this case, the training of the first neural network model may include validating the first sub-neural network model using the first validation data set to obtain accuracy of the first sub-neural network model, validating the second sub-neural network model using the first validation data set to obtain accuracy of the second sub-neural network model, and comparing the accuracy of the first sub-neural network model and the accuracy of the second sub-neural network model to determine a more accurate sub-neural network model as the final neural network model.

### 1.3. Diagnosis Assistance Process

According to an embodiment of the present invention, a diagnosis assistance process (or diagnostic process) in which diagnosis assistance information is obtained using a neural network model may be provided. As a specific example, by the diagnosis assistance process, diagnosis assistance information (for example, diagnostic information or findings information) may be predicted through a diagnosis assistance neural network model trained using a fundus image.

The diagnosis assistance process which will be described below may be performed by a diagnostic device.

### 1.3.1 Diagnostic Unit

According to an embodiment of the present invention, a diagnostic process may be performed by a diagnostic unit 200. The diagnostic unit 200 may be provided in the above-described diagnostic device.

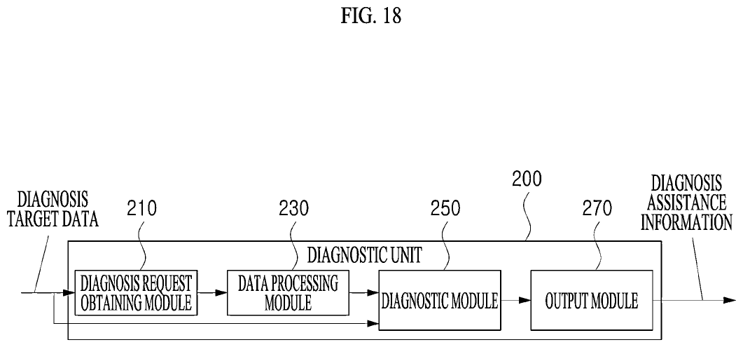

FIG. 18 is a view for describing a configuration of the diagnostic unit 200 according to an embodiment of the present invention. Referring to FIG. 18, the diagnostic unit 200 may include a diagnosis request obtaining module 210, a data processing module 230, a diagnostic module 250, and an output module 270.

As will be described below, the modules may perform individual steps of a data processing process and a training process. However, not all of the elements described with reference to FIG. 18 and functions performed by the elements are essential, and some elements may be added or omitted according to an aspect of diagnosis.

### 1.3.2 Obtaining Data and Diagnosis Request

The diagnostic device according to an embodiment of the present invention may obtain diagnosis target data and obtain diagnosis assistance information based on the obtained diagnosis target data. The diagnosis target data may be image data. The obtaining of the data and obtaining of a diagnosis request may be performed by the diagnosis request obtaining module of the above-described diagnostic unit.

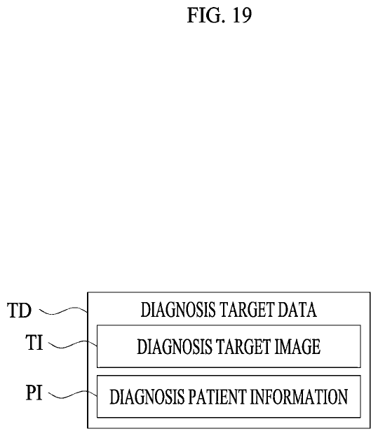

FIG. 19 is a view for describing diagnosis target data TD according to an embodiment of the present invention. Referring to FIG. 19, the diagnosis target data TD may include a diagnosis target image TI and diagnosis target subject information PI.

The diagnosis target image TI may be an image for obtaining diagnosis assistance information on a diagnosis target subject. For example, the diagnosis target image may be a fundus image. The diagnosis target image TI may have any one format among JPG, PNG, DCM (DICOM), BMP, GIF, and TIFF.

The diagnosis subject information PI may be information for identifying a subject to be diagnosed. Alternatively, the diagnosis subject information PI may be characteristic information of a subject or an image to be diagnosed. For example, the diagnosis subject information PI may include information such as the date and time of imaging and imaging equipment of an image to be diagnosed or information such as an identification (ID) number, an ID, name, age, or weight of a subject to be diagnosed. When the image to be diagnosed is a fundus image, the diagnosis subject information PI may further include eye-related information such as left eye/right eye information on whether the corresponding fundus image is an image of the left eye or an image of the right eye.

The diagnostic device may obtain a diagnosis request. The diagnostic device may obtain diagnosis target data together with the diagnosis request. When the diagnosis request is obtained, the diagnostic device may obtain diagnosis assistance information using a trained diagnosis assistance neural network model. The diagnostic device may obtain a diagnosis request from a client device. Alternatively, the diagnostic device may obtain a diagnosis request from a user through a separately-provided input means.

### 1.3.3 Date Processing Process

Obtained data may be processed. Data processing may be performed by the data processing module of the above-described diagnostic unit.

Generally, a data processing process may be performed similar to the data processing process in the above-described training process. Hereinafter, the data processing process in the diagnostic process will be described focusing on differences from the data processing process in the training process.

In the diagnostic process, the diagnostic device may obtain data as in the training process. In this case, the obtained data may have the same format as the data obtained in the training process. For example, when the training device has trained a diagnosis assistance neural network model using image data in the DCM format in the training process, the diagnostic device may obtain the DCM image and obtain diagnosis assistance information using the trained neural network model.

In the diagnostic process, the obtained image to be diagnosed may be resized similar to the image data used in the training process. To efficiently perform prediction of diagnosis assistance information through the trained diagnosis assistance neural network model, the form of the image to be diagnosed may be adjusted to have a suitable volume, size, and/or aspect ratio.

For example, when an image to be diagnosed is a fundus image, resizing of the image such as removing an unnecessary portion of the image or reducing the size of the image may be performed to predict diagnostic information based on the fundus image.

In the diagnostic process, similar to the image data used in the training process, a pre-processing filter may be applied to the obtained image to be diagnosed. A suitable filter may be applied to the image to be diagnosed so that accuracy of prediction of diagnosis assistance information through a trained diagnosis assistance neural network model is further improved.

For example, when an image to be diagnosed is a fundus image, pre-processing that facilitates prediction of correct diagnostic information, for example, image pre-processing that causes a blood vessel to be emphasized or image pre-processing that causes a specific color to be emphasized or weakened, may be applied to the image to be diagnosed.

In the diagnostic process, similar to the image data used in the training process, the obtained image to be diagnosed may be serialized. The image to be diagnosed may be converted to a form that facilitates driving of a diagnostic model in a specific work frame or may be serialized.

The serializing of the image to be diagnosed may be omitted. This may be because, in the diagnostic process, the number of data processed at one time by a processor is not large unlike in the training process, and thus the burden on data processing speed is relatively small.

In the diagnostic process, similar to the image data used in the training process, the obtained image to be diagnosed may be stored in a queue. However, since the number of data being processed is smaller in the diagnostic process in comparison to that in the training process, storing data in a queue may also be omitted.

Meanwhile, since an increase in the number of data is not required in the diagnostic process, it is preferable that, in order to obtain accurate diagnosis assistance information, the process of data augmentation or image augmentation is not used, unlike in the training process.

### 1.3.4 Diagnostic Process

According to an embodiment of the present invention, a diagnostic process using a trained neural network model may be disclosed. The diagnostic process may be performed by the above-described diagnostic device. The diagnostic process may be performed by the above-described diagnostic server. The diagnostic process may be performed by the control unit of the above-described diagnostic device. The diagnostic process may be performed by the diagnostic module of the above-described diagnostic unit.

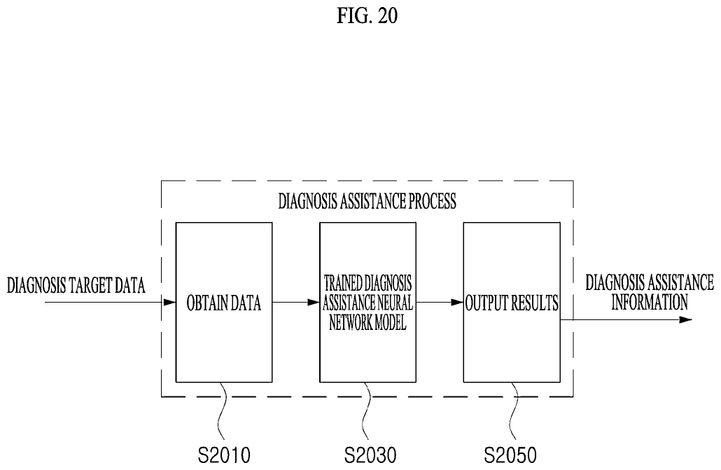

FIG. 20 is a view for describing a diagnostic process according to an embodiment of the present invention. Referring to FIG. 20, the diagnostic process may include obtaining diagnosis target data (S2010), using a trained neural network model (S2030), and obtaining and outputting a result corresponding to the obtained diagnosis target data (S2050). However, data processing may be selectively performed.

Hereinafter, each step of the diagnostic process will be described with reference to FIG. 20.

### 1.3.4.1 Data Input

According to an embodiment of the present invention, the diagnostic module may obtain diagnosis target data. The obtained data may be data processed as described above. For example, the obtained data may be a subject's fundus image data to which pre-processing that causes the size to be adjusted and a blood vessel to be emphasized is applied. According to an embodiment of the present invention, a left eye image and a right eye image of a single subject may be input together as diagnosis target data.

### 1.3.4.2 Data Classification

A diagnosis assistance neural network model provided in the form of a classifier may classify input diagnosis target images into a positive class or a negative class in relation to a predetermined label.

A trained diagnosis assistance neural network model may receive diagnosis target data and output a predicted label. The trained diagnosis assistance neural network model may output a predicted value of diagnosis assistance information. Diagnosis assistance information may be obtained using the trained diagnosis assistance neural network model. The diagnosis assistance information may be determined based on the predicted label.

For example, the diagnosis assistance neural network model may predict diagnostic information (that is, information on the presence of a disease) or findings information (that is, information on the presence of abnormal findings) related to an eye disease or a systemic disease of the subject. In this case, the diagnostic information or findings information may be output in the form of a probability. For example, the probability that the subject has a specific disease or the probability that there may be a specific abnormal finding in the subject's fundus image may be output. When a diagnosis assistance neural network model provided in the form of a classifier is used, a predicted label may be determined in consideration of whether an output probability value (or predicted score) exceeds a threshold value.

As a specific example, a diagnosis assistance neural network model may output a probability value with respect to the presence of diabetic retinopathy in a subject with the subject's fundus image as a diagnosis target image. When a diagnosis assistance neural network model in the form of a classifier that assumes 1 as normal is used, a subject's fundus image may be input to the diagnosis assistance neural network model, and in relation to whether the subject has diabetic retinopathy, a normal: abnormal probability value may be obtained in the form of 0.74:0.26 or the like.

Although the case in which data is classified using the diagnosis assistance neural network model in the form of a classifier has been described herein, the present invention is not limited thereto, and a specific diagnosis assistance numerical value (for example, blood pressure or the like) may also be predicted using a diagnosis assistance neural network model implemented in the form of a regression model.

According to another embodiment of the present invention, suitability information on an image may be obtained. The suitability information may indicate whether a diagnosis target image is suitable for obtaining diagnosis assistance information using a diagnosis assistance neural network model.

The suitability information of an image may be quality information. The quality information or suitability information may indicate whether a diagnosis target image reaches a reference level.

For example, when a diagnosis target image has a defect due to a defect of imaging equipment or an influence of an illumination during imaging, a result indicating that the diagnosis target image is unsuitable may be output as suitability information of the corresponding diagnosis target image. When a diagnosis target image includes noise at a predetermined level or higher, the diagnosis target image may be determined as being unsuitable.

The suitability information may be a value predicted using a neural network model. Alternatively, the suitability information may be information obtained through a separate image analysis process.

According to an embodiment, even when an image is classified as unsuitable, diagnosis assistance information may be obtained based on the unsuitable image.

According to an embodiment, an image classified as unsuitable may be reexamined by a diagnosis assistance neural network model.

In this case, the diagnosis assistance neural network model that performs the reexamination may differ from a diagnosis assistance neural network model that performs initial examination. For example, the diagnostic device may store a first diagnosis assistance neural network model and a second diagnosis assistance neural network model, and an image classified as unsuitable through the first diagnosis assistance neural network model may be examined through the second diagnosis assistance neural network model.

According to still another embodiment of the present invention, a class activation map (CAM) may be obtained from a trained neural network model. Diagnosis assistance information may include a CAM. The CAM may be obtained together with other diagnosis assistance information.

The CAM may be obtained optionally. For example, the CAM may be extracted and/or output when diagnostic information or findings information obtained by a diagnosis assistance model is classified into an abnormal class.

### 1.3.5 Output of Diagnosis Assistance Information

Diagnosis assistance information may be determined based on a label predicted from a diagnosis assistance neural network model.

Output of diagnosis assistance information may be performed by the output module of the above-described diagnostic unit. Diagnosis assistance information may be output from the diagnostic device to a client device. Diagnosis assistance information may be output from the diagnostic device to a server device. Diagnosis assistance information may be stored in the diagnostic device or diagnostic server. Diagnosis assistance information may be stored in a separately-provided server device or the like.

Diagnosis assistance information may be managed by being formed into a database. For example, obtained diagnosis assistance information may be stored and managed together with a diagnosis target image of a subject according to an identification number of the corresponding subject. In this case, the diagnosis target image and diagnosis assistance information of the subject may be managed in chronological order. By managing the diagnosis assistance information and diagnosis target image in time series, tracking personal diagnostic information and managing history thereof may be facilitated.

Diagnosis assistance information may be provided to a user. The diagnosis assistance information may be provided to the user through an output means of a diagnostic device or client device. The diagnosis assistance information may be output through a visual or aural output means provided in the diagnostic device or client device so that the user may recognize the diagnosis assistance information.

According to an embodiment of the present invention, an interface for effectively providing diagnosis assistance information to a user may be provided. Such a user interface will be described in more detail below in Section “5. User interface.”

When a CAM is obtained by a neural network model, an image of the CAM may be provided together. The image of the CAM may be selectively provided. For example, the CAM image may not be provided when diagnostic information obtained through a diagnosis assistance neural network model is normal findings information or normal diagnostic information, and the CAM image may be provided together for more accurate clinical diagnosis when the obtained diagnostic information is abnormal findings information or abnormal diagnostic information.

When an image is classified as unsuitable, suitability information of the image may be provided together. For example, when an image is classified as unsuitable, diagnosis assistance information and “unsuitable” judgment information obtained according to the corresponding image may be provided together.

A diagnosis target image that has been judged to be unsuitable may be classified as an image to be retaken. In this case, a retake guide for a target subject of the image classified as an image to be retaken may be provided together with the suitability information.

Meanwhile, in response to providing of diagnosis assistance information obtained through a neural network model, feedback related to training of the neural network model may be obtained. For example, feedback for adjusting a parameter or hyperparameter related to training of the neural network model may be obtained. The feedback may be obtained through a user input unit provided in the diagnostic device or client device.

According to an embodiment of the present invention, diagnosis assistance information corresponding to a diagnosis target image may include level information. The level information may be selected among a plurality of levels. The level information may be determined based on diagnostic information and/or findings information obtained through a neural network model. The level information may be determined in consideration of suitability information or quality information of a diagnosis target image. When a neural network model is a classifier model that performs multiclass classification, the level information may be determined in consideration of a class into which a diagnosis target image is classified by the neural network model. When a neural network model is a regression model that outputs a numerical value related to a specific disease, the level information may be determined in consideration of the output numerical value.

For example, diagnosis assistance information obtained corresponding to a diagnosis target image may include any one level information selected from a first level information and a second level information. When abnormal findings information or abnormal diagnostic information is obtained through a neural network model, the first level information may be selected as the level information. When abnormal findings information or abnormal diagnostic information is not obtained through a neural network model, the second level information may be selected as the level information. Alternatively, the first level information may be selected as the level information when a numerical value obtained through a neural network model exceeds a reference numerical value, and the second level information may be selected as the level information when the obtained numerical value is less than the reference numerical value. The first level information may indicate that strong abnormal information is present in a diagnosis target image compared with the second level information.

Meanwhile, a third level information may be selected as the level information when the quality of a diagnosis target image is determined to a reference quality or lower using image analysis or a neural network model. Alternatively, diagnosis assistance information may include the third level information together with the first or second level information.

When diagnosis assistance information includes the first level information, a first user guide may be output through an output means. The first user guide may indicate that a more precise test is required for a subject (i.e. patient) corresponding to the diagnosis assistance information. For example, the first user guide may indicate that secondary diagnosis (for example, diagnosis in a separate medical institution or a hospital transfer procedure) is required for the subject. Alternatively, the first user guide may indicate treatment required for the subject. As a specific example, when abnormal information on macular degeneration of the subject is obtained by diagnosis assistance information, the first user guide may include injection prescription and a guide on a hospital transfer procedure (for example, a list of hospitals to which transfer is possible) related to the subject.

When diagnosis assistance information includes the second level information, a second user guide may be output through an output means. The second user guide may include future care plans related to the subject corresponding to the diagnosis assistance information. For example, the second user guide may indicate the time of next visit and the next medical course.

When diagnosis target information includes the third level information, a third user guide may be output through an output means. The third user guide may indicate that a diagnosis target image has to be retaken. The third user guide may include information on the quality of the diagnosis target image. For example, the third user guide may include information on an artifact present in a diagnosis target image (for example, whether the artifact is a bright artifact or a dark artifact, or the degree thereof).

### 1.4 Diagnosis Assistance System for Multiple Labels

According to an embodiment of the present invention, a diagnosis assistance system for performing prediction on a plurality of labels (for example, a plurality of diagnosis assistance information) may be provided. For this, a diagnosis assistance neural network of the above-mentioned diagnosis assistance system may be designed to perform prediction on a plurality of labels.

Alternatively, in the above-mentioned diagnosis assistance system, a plurality of diagnosis assistance neural networks that perform prediction on different labels may be used in parallel. Hereinafter, such a parallel diagnosis assistance system will be described.

### 1.4.1 Configuration of Parallel Diagnosis Assistance System

According to an embodiment of the present invention, a parallel diagnosis assistance system for obtaining a plurality of diagnosis assistance information may be provided. The parallel diagnosis assistance system may train a plurality of neural network models for obtaining a plurality of diagnosis assistance information and obtain the plurality of diagnosis assistance information using the trained plurality of neural network models.

For example, the parallel diagnosis assistance system may train, based on fundus images, a first neural network model that obtains a first diagnosis assistance information related to the presence of an eye disease of a subject and a second neural network model that obtains a second diagnosis assistance information related to the presence of a systemic disease of the subject and may output the diagnosis assistance information related to the presence of an eye disease and the presence of a systemic disease of the subject by using the trained first neural network model and the second neural network model.

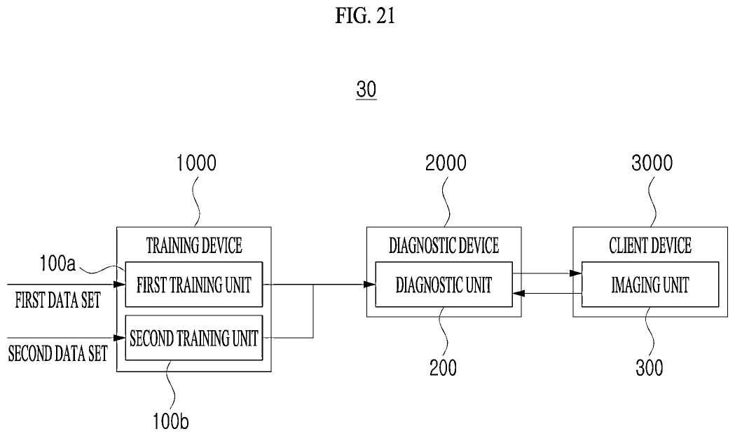

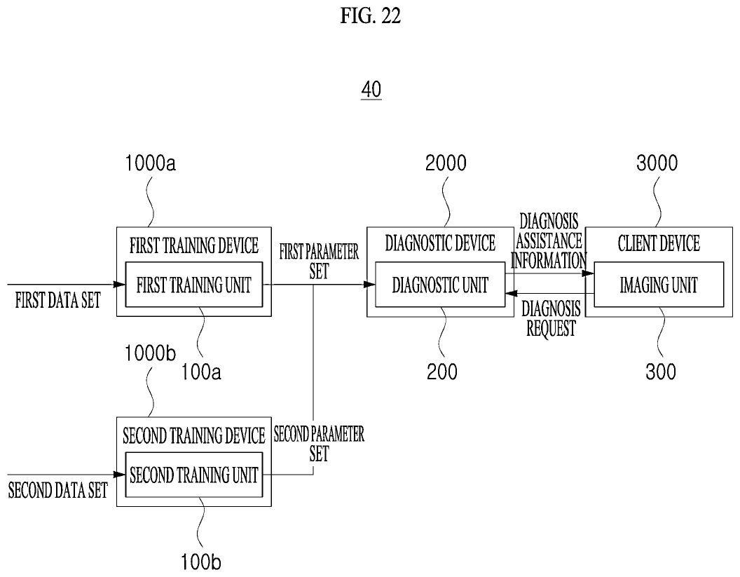

FIGS. 21 and 22 are views for describing a parallel diagnosis assistance system according to some embodiments of the present invention. Referring to FIGS. 21 and 22, the parallel diagnosis assistance system may include a plurality of training units.

Referring to FIG. 21, a parallel diagnosis assistance system 30 according to an embodiment of the present invention may include a training device 1000, a diagnostic device 2000, and a client device 3000. In this case, the training device 1000 may include a plurality of training units. For example, the training device 1000 may include a first training unit 100a and a second training unit 100b.

Referring to FIG. 22, a parallel diagnosis assistance system 40 according to an embodiment of the present invention may include a first training device 1000a, a second training device 1000b, a diagnostic device 2000, and a client device 3000. The first training device 1000a may include a first training unit 100a. The second training device 1000b may include a second training unit 100b.

Referring to FIGS. 21 and 22, the first training unit 100a may obtain a first data set and output a first parameter set of a first neural network model obtained as a result of training the first neural network model. The second training unit 100b may obtain a second data set and output a second parameter set of a second neural network model obtained as a result of training the second neural network model.

The diagnostic device 2000 may include a diagnostic unit 200. Description similar to that given above with reference to FIG. 1 may be applied to the diagnostic device 2000 and the diagnostic unit 200. The diagnostic unit 200 may obtain a first diagnosis assistance information and a second diagnosis assistance information using the trained first neural network model and second neural network model through the first training unit 100a and the second training unit 100b. The diagnostic unit 200 may store parameters of the trained first neural network model and parameters of the trained second neural network model obtained from the first training unit 100a and the second training unit 100b.

The client device 3000 may include a data obtaining unit, e.g., an imaging unit 300. However, the imaging unit 300 may be substituted with other data obtaining means used for obtaining diagnosis assistance information. The client device may transmit a diagnosis request and diagnosis target data (for example, a fundus image obtained by the imaging unit) to the diagnostic device. In response to the transmitting of the diagnosis request, the client device 3000 may obtain, from the diagnostic device, a plurality of diagnosis assistance information according to the transmitted diagnosis target data.

Meanwhile, although the case in which the diagnosis assistance system 40 includes the first training unit 100a and the second training unit 100b has been described above with reference to FIGS. 21 and 22, content of the invention is not limited thereto. According to another embodiment of the present invention, a training device may include a training unit configured to obtain three or more different diagnosis assistance information. Alternatively, a diagnosis assistance system may also include a plurality of training devices configured to obtain different diagnosis assistance information

The operations of the training device, the diagnostic device, and the client device will be described in more detail below.

### 1.4.2 Parallel Training Process

According to an embodiment of the present invention, a plurality of neural network models may be trained. Training processes for training the respective neural network models may be performed in parallel.

### 1.4.2.1 Parallel Training Units

Training processes may be performed by a plurality of training units. The training processes may be performed independently of each other. The plurality of training units may be provided in a single training device or respectively provided in a plurality of training devices.

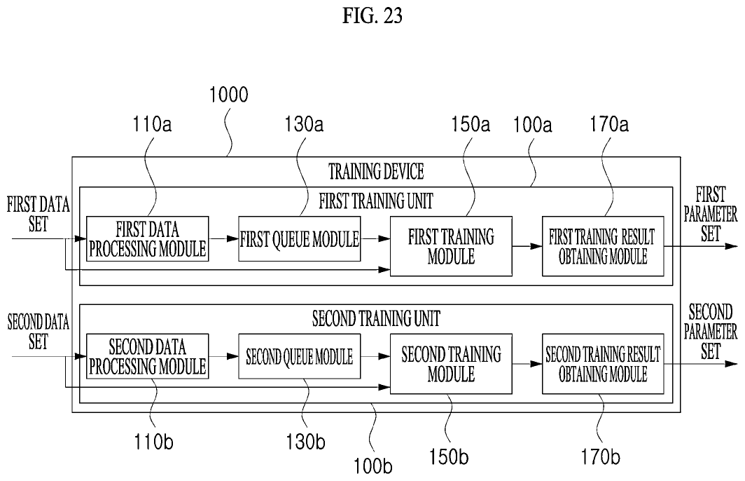

FIG. 23 is a view for describing a configuration of a training device including a plurality of training units according to an embodiment of the present invention. The configuration and operation of each of the first training unit 100a and the second training unit 100b may be implemented similar to those described above with reference to FIG. 9.

Referring to FIG. 23, a process of a neural network model according to an embodiment of the present invention may be performed by a training device 1000 including a first training unit 100a which includes a first data processing module 110a, a first queue module 130a, a first training module 150a, and a first training result obtaining module 170a and a second training unit 100b which includes a second data processing module 110b, a second queue module 130b, a second training module 150b, and a second training result obtaining module 170b.

Referring to FIG. 23, a training process of a neural network model according to an embodiment of the present invention may be performed by each of the first training unit 100a and the second training unit 100b. The first training unit 100a and the second training unit 100b may independently perform training of the first neural network model and the second neural network model. Referring to FIG. 23, the first training unit 100a and the second training unit 100b may be provided in the above-described training device. Alternatively, the first training unit and the second training unit may also be provided in different training devices.

### 1.4.2.2 Obtaining Parallel Data

According to an embodiment of the present invention, a plurality of training units may obtain data. The plurality of training units may obtain different data sets. Alternatively, the plurality of training units may also obtain the same data set. According to circumstances, the plurality of training units may also obtain partially common data sets. The data sets may be fundus image data sets.

A first training unit may obtain a first data set, and a second training unit may obtain a second data set. The first data set and the second data set may be distinguished from each other. The first data set and the second data set may be partially common. The first data set and the second data set may be labeled fundus image data sets.

The first data set may include data labeled as normal in relation to a first feature and data labeled as abnormal in relation to the first feature. For example, the first data set may include a fundus image labeled as normal and a fundus image labeled as abnormal in relation to the opacity of crystalline lens.

The second data set may include data labeled as normal in relation to a second feature (distinguished from the first feature) and data labeled as abnormal in relation to the second feature. For example, the second data set may include a fundus image labeled as normal and a fundus image labeled as abnormal in relation to diabetic retinopathy.

The data labeled as normal in relation to the first feature and data labeled as normal in relation to the second feature respectively included in the first data set and the second data set may be common. For example, the first data set may include a fundus image labeled as normal and a fundus image labeled as abnormal in relation to the opacity of crystalline lens, and the second data set may include a fundus image labeled as normal and a fundus image labeled as abnormal in relation to diabetic retinopathy, wherein the fundus image labeled as normal in relation to the opacity of crystalline lens included in the first data set and the fundus image labeled as normal in relation to diabetic retinopathy included in the second data set may be common.

Alternatively, the data labeled as abnormal in relation to the first feature and the data labeled as abnormal in relation to the second feature respectively included in the first data set and the second data set may also be common. That is, data labeled in relation to a plurality of features may be used in training a neural network model in relation to the plurality of features.

Meanwhile, the first data set may be a fundus image data set captured using a first method, and the second data set may be a fundus image data set captured using a second method. The first method and the second method may be any one method selected from red-free imaging, panoramic imaging, autofluorescence imaging, infrared imaging, and the like.

A data set used in each training unit may be determined in consideration of diagnosis assistance information obtained by a trained neural network model. For example, when the first training unit trains a first neural network model which desires to obtain diagnosis assistance information related to abnormal findings of the retina (for example, microaneurysms, exudates, and the like), the first training unit may obtain a first fundus image data set captured by red-free imaging. Alternatively, when the second training unit trains a second neural network model which desires to obtain diagnosis assistance information related to macular degeneration, the second training unit may obtain a second fundus image data set captured by autofluorescence imaging.

### 1.4.3 Parallel Data Processing

The plurality of training units may process obtained data. As described above in Section “2.2 Data processing process,” each training unit may process data by applying one or more of image resizing, a pre-processing filter, image augmentation, and image serialization to obtained data. The first data processing module of the first training unit may process a first data set, and the second data processing module of the second training unit may process a second data set.

The first training unit and second training unit included in the plurality of training units may differently process obtained data sets in consideration of diagnosis assistance information obtained from neural network models respectively trained by the first training unit and the second training unit. For example, to train a first neural network model for obtaining a first diagnosis assistance information related to hypertension, the first training unit may perform pre-processing that causes blood vessels to be emphasized in fundus images included in the first fundus image data set. Alternatively, to train a second neural network model for obtaining a second diagnosis assistance information related to abnormal findings on exudates, microaneurysms, and the like of the retina, the second training unit may perform pre-processing that causes fundus images included in the second fundus image data set to be converted to red-free images.

### 1.4.3.1 Parallel Queue

The plurality of training units may store data in a queue. As described above in Section “2.2.6 Queue,” each training unit may store processed data in a queue and transmit the processed data to the training module. For example, the first training unit may store a first data set in a first queue module and provide the first data set to a first training module sequentially or randomly. The second training module may store a second data set in a second queue module and provide the second data set to a second training module sequentially or randomly.

### 1.4.3.2 Parallel Training Process

The plurality of training units may train a neural network model. The training modules may independently train diagnosis assistance neural network models that perform prediction on different labels using training data sets. A first training module of the first training unit may train the first neural network model, and a second training module of the second training unit may train the second neural network model.

The plurality of diagnosis assistance neural network models may be trained in parallel and/or independently. By training models to perform prediction on different labels through the plurality of neural network models in this way, accuracy of prediction on each label may be improved, and efficiency of the prediction operation may be enhanced.

Each diagnosis assistance neural network model may be provided similar to that described above in Section “2.3.2 Model design.” Each sub-training process may be performed similar to that described above in Sections 2.3.1 to 2.3.5.

A parallel training process according to an embodiment of the present invention may include training diagnosis assistance neural network models that predict different labels. The first training unit may train a first diagnosis assistance neural network model that predicts a first label. The second training unit may train a second diagnosis assistance neural network model that predicts a second label.

The first training unit may obtain a first data set and train the first diagnosis assistance neural network model that predicts the first label. For example, the first training unit may train the first diagnosis assistance neural network model that predicts the presence of macular degeneration of a subject from a fundus image by using a fundus image training data set labeled in relation to the presence of macular degeneration.

The second training unit may obtain a second data set and train the second diagnosis assistance neural network model that predicts the second label. For example, the second training unit may train the second diagnosis assistance neural network model that predicts the presence of diabetic retinopathy of a subject from a fundus image by using a fundus image training data set labeled in relation to the presence of diabetic retinopathy.

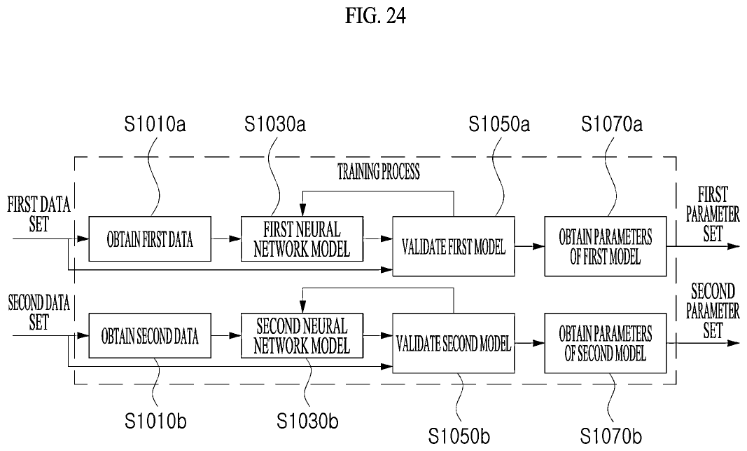

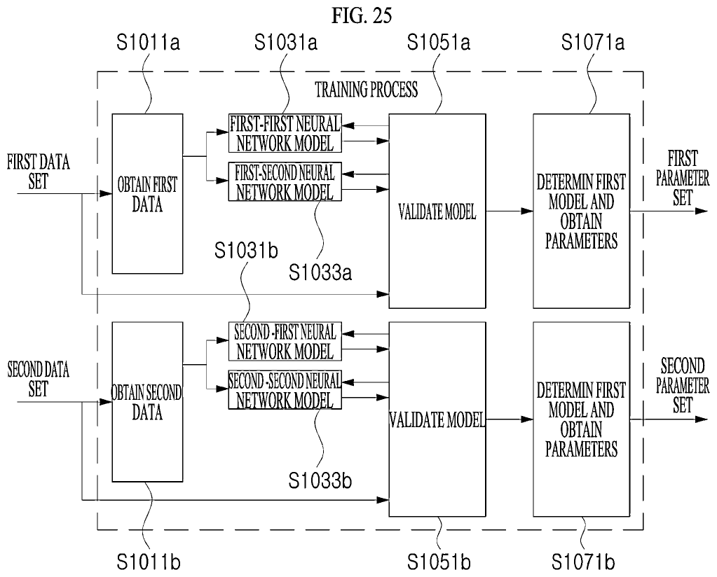

The training process of a neural network model will be described in more detail below with reference to FIGS. 24 and 25.

FIG. 24 is a view for describing a parallel training process according to an embodiment of the present invention. The parallel training process may be applied to all of the cases in which the parallel diagnosis assistance system is implemented as shown in FIG. 21, implemented as shown in FIG. 22, and implemented in other forms. However, for convenience of description, description will be given below based on the parallel diagnosis assistance system implemented as shown in FIG. 21.

Referring to FIG. 24, the parallel training process may include a plurality of sub-training processes that respectively train a plurality of diagnosis assistance neural network models that predict different labels. The parallel training process may include a first sub-training process that trains a first neural network model and a second sub-training process that trains a second neural network model.

For example, the first sub-training process may be performed by obtaining a first data (S1010a), using a first neural network model (S1030a), validating the first model (that is, first diagnosis assistance neural network model) (S1050a), and obtaining parameters of the first neural network model (S1070a). The second sub-training process may be performed by obtaining a second data (S1010b), using a second neural network model (1030b), validating the second neural network model (that is, second diagnosis assistance neural network model) (S1050b), and obtaining parameters of the second neural network model (S1070b).

A sub-training process may include training a neural network model by inputting training data into a sub-neural network model, comparing a label value obtained by output with the input training data to validate the model, and reflecting a validation result back to the sub-neural network model.

Each sub-training process may include obtaining result values using a neural network model to which arbitrary weight values are assigned, comparing the obtained result values with label values of training data, and performing backpropagation according to errors therebetween to optimize the weight values.

In each sub-training process, a diagnosis assistance neural network model may be validated through a validation data set distinguished from a training data set. Validation data sets for validating a first neural network model and a second neural network model may be distinguished.

The plurality of training units may obtain training results. Each training result obtaining module may obtain information on neural network models trained from the training modules. Each training result obtaining module may obtain parameter values of neural network models trained from the training units. A first training result obtaining module of the first training unit may obtain a first parameter set of a first neural network model trained from a first training module. A second training result obtaining module of the second training unit may obtain a second parameter set of a second neural network model trained from a second training module.

By each sub-training process, optimized parameter values, that is, a parameter set, of a trained neural network model may be obtained. As training is performed using more training data sets, more suitable parameter values may be obtained.

A first parameter set of a first diagnosis assistance neural network model trained by a first sub-training process may be obtained. A second parameter set of a second diagnosis assistance neural network model trained by a second sub-training process may be obtained. As training is sufficiently performed, optimized values of weights and/or bias of the first diagnosis assistance neural network and the second diagnosis assistance neural network may be obtained.

The obtained parameter set of each neural network model may be stored in the training device and/or the diagnostic device (or server). The first parameter set of the first diagnosis assistance neural network and the second parameter set of the second diagnosis assistance neural network may be stored together or separately. A parameter set of each trained neural network model may also be updated by feedback obtained from the diagnostic device or client device.

### 1.4.3.3 Parallel Ensemble Training Process

Even when a plurality of neural network models are trained in parallel, The above-described ensemble form of model training may be used. Each sub-training process may include training a plurality of sub-neural network models. The plurality of sub-models may have different layer structures. Hereinafter, unless particularly mentioned otherwise, description similar to that given above in Section 2.3.7 may be applied.

When a plurality of diagnosis assistance neural network models are trained in parallel, some sub-training processes among the sub-training processes that train the diagnosis assistance neural network models may train a single model, and other sub-training processes may train a plurality of sub-models together.

Since models are trained using ensembles in each sub-training process, more optimized forms of neural network models may be obtained in each sub-training process, and error in prediction may be reduced.

FIG. 25 is a view for describing the parallel training process according to another embodiment of the present invention. Referring to FIG. 25, each training process may include training a plurality of sub-neural network models.

Referring to FIG. 25, a first sub-training process may be performed by obtaining a first data S1011a, using a first-first (1-1) neural network model and a first-second (1-2) neural network model (S1031a, S1033a), validating the first-first (1-1) neural network model and the first-second (1-2) neural network model (S1051a), and determining a final form of the first neural network model and parameters thereof (S1071a). A second sub-training process may be performed by obtaining a second data (S1011b), using a second-first (2-1) neural network model and a second-second (2-2) neural network model (S1031b, S1033b), validating the second-first (2-1) neural network model and the second-second (2-2) neural network model (S1051b), and determining a final form of the second model (that is, the second diagnosis assistance neural network model) and parameters thereof (S1071b).

The first neural network trained in the first sub-training process may include the first-first (1-1) neural network model and the first-second (1-2) neural network model. The first-first (1-1) neural network model and the first-second (1-2) neural network model may be provided in different layer structures. Each of the first-first (1-1) neural network model and the first-second (1-2) neural network model may obtain a first data set and output predicted labels. Alternatively, a label predicted by an ensemble of the first-first (1-1) neural network model and the first-second (1-2) neural network model may be determined as a final predicted label.

In this case, the first-first (1-1) neural network model and the first-second (1-2) neural network model may be validated using a validation data set, and a more accurate neural network model may be determined as a final neural network model. Alternatively, the first-first (1-1) neural network model, the first-second (1-2) neural network model, and the ensemble of the first-first (1-1) neural network model and the first-second (1-2) neural network model may be validated, and a neural network model form of the most accurate case may be determined as a final first neural network model.

For the second sub-training process, likewise, the most accurate form of neural network among the second-first (2-1) neural network model, the second-second (2-2) neural network model, and the ensemble of the second-first (2-1) neural network model and the second-second (2-2) neural network model may be determined as the final second model (that is, second diagnosis assistance neural network model).

Meanwhile, although, for convenience of description, the case in which each sub-training process includes two sub-models has been described above with reference to FIG. 25, this is merely an example, and the present invention is not limited thereto. A neural network model trained in each sub-training process may only include a single neural network model or include three or more sub-models.

### 1.4.4 Parallel Diagnostic Process

According to an embodiment of the present invention, a diagnostic process for obtaining a plurality of diagnosis assistance information may be provided. The diagnostic process for obtaining the plurality of diagnosis assistance information may be implemented in the form of a parallel diagnosis assistance process including a plurality of diagnostic processes which are independent from each other.

### 1.4.4.1 Parallel Diagnostic Unit

According to an embodiment of the present invention, a diagnosis assistance process may be performed by a plurality of diagnostic modules. Each diagnosis assistance process may be independently performed.

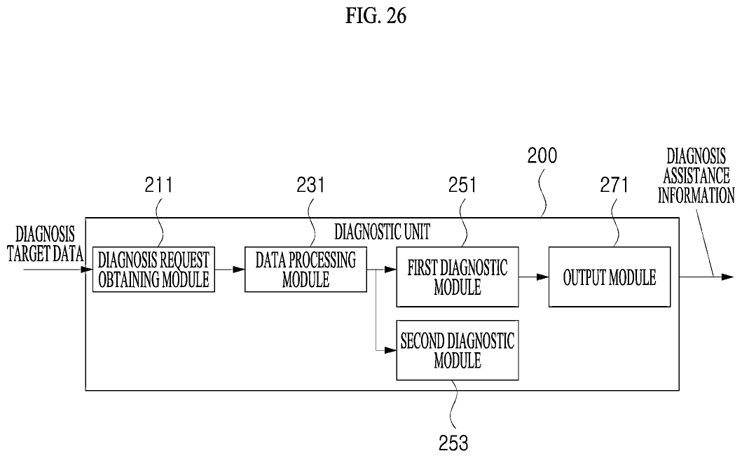

FIG. 26 is a block diagram for describing a diagnostic unit 200 according to an embodiment of the present invention.

Referring to FIG. 26, the diagnostic unit 200 according to an embodiment of the present invention may include a diagnosis request obtaining module 211, a data processing module 231, a first diagnostic module 251, a second diagnostic module 253, and an output module 271. Unless particularly mentioned otherwise, each module of the diagnostic unit 200 may operate similar to the diagnostic module of the diagnostic unit illustrated in FIG. 18.

In FIG. 26, the diagnosis request obtaining module 211, the data processing module 231, and the output module 271 have been illustrated as being common even when the diagnostic unit 200 includes a plurality of diagnostic modules, but the present invention is not limited to such a configuration. The diagnosis request obtaining module, the data processing module, and/or the output module may also be provided in plural. The plurality of diagnosis request obtaining modules, data processing modules, and/or output modules may also operate in parallel.

For example, the diagnostic unit 200 may include a first data processing module configured to perform first processing of an input diagnosis target image and a second data processing module configured to perform second processing of the diagnosis target image, the first diagnostic module may obtain a first diagnosis assistance information based on the diagnosis target image on which the first processing has been performed, and the second diagnostic module may obtain a second diagnosis assistance information based on the diagnosis target image on which the second processing has been performed. The first processing and/or second processing may be any one selected from image resizing, image color modulation, blur filter application, blood vessel emphasizing process, red-free conversion, partial region cropping, and extraction of some elements.

The plurality of diagnostic modules may obtain different diagnosis assistance information. The plurality of diagnostic modules may obtain diagnosis assistance information using different diagnosis assistance neural network models. For example, the first diagnostic module may obtain a first diagnosis assistance information related to the presence of an eye disease of a subject by using a first neural network model that predicts the presence of an eye disease of the subject, and the second diagnostic module may obtain a second diagnosis assistance information related to the presence of a systemic disease of a subject by using a second neural network model that predicts the presence of a systemic disease of the subject.

As a more specific example, the first diagnostic module may obtain a first diagnosis assistance information related to the presence of diabetic retinopathy of the subject using a first diagnosis assistance neural network model that predicts the presence of diabetic retinopathy of the subject, and the second diagnostic module may obtain a second diagnosis assistance information related to the presence of hypertension using a second diagnosis assistance neural network model that predicts the presence of hypertension of the subject.

### 1.4.4.2 Parallel Diagnostic Process

A diagnosis assistance process according to an embodiment of the present invention may include a plurality of sub-diagnostic processes. Each sub-diagnostic process may be performed using different diagnosis assistance neural network models. Each sub-diagnostic process may be performed in different diagnostic modules. For example, a first diagnostic module may perform a first sub-diagnostic process that obtains a first diagnosis assistance information through a first diagnosis assistance neural network model. Alternatively, a second diagnostic module may perform a second sub-diagnostic process that obtains a second diagnosis assistance information through a second diagnosis assistance neural network model.

The plurality of trained neural network models may output a predicted label or probability with diagnosis target data as input. Each neural network model may be provided in the form of a classifier and may classify input diagnosis target data as a predetermined label. In this case, the plurality of neural network models may be provided in forms of classifiers that are trained in relation to different characteristics. Each neural network model may classify diagnosis target data as described above in Section 3.4.2.

Meanwhile, a CAM may be obtained from each diagnosis assistance neural network model. The CAM may be obtained selectively. The CAM may be extracted when a predetermined condition is satisfied. For example, when a first diagnosis assistance information indicates that the subject is abnormal in relation to a first characteristic, a first CAM may be obtained from a first diagnosis assistance neural network model.

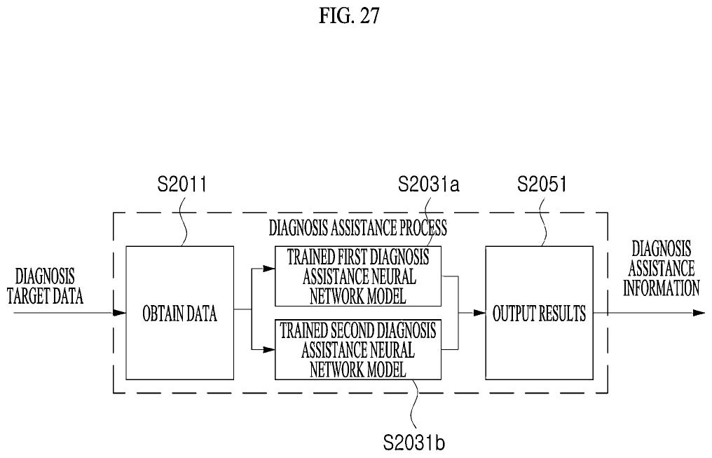

FIG. 27 is a view for describing a diagnosis assistance process according to an embodiment of the present invention.

Referring to FIG. 27, a diagnosis assistance process according to an embodiment of the present invention may include obtaining diagnosis target data (S2011), using a first diagnosis assistance neural network model and a second diagnosis assistance neural network model (52031a, 52031b), and obtaining diagnosis assistance information according to diagnosis target data (S2051). The diagnosis target data may be processed data.

The diagnosis assistance process according to an embodiment of the present invention may include obtaining a first diagnosis assistance information through the trained first diagnosis assistance neural network model and obtaining a second diagnosis assistance information through the trained second diagnosis assistance neural network model. The first diagnosis assistance neural network model and the second diagnosis assistance neural network model may obtain the first diagnosis assistance information and the second diagnosis assistance information respectively, based on the same diagnosis target data.

For example, the first diagnosis assistance neural network model and the second diagnosis assistance neural network model may respectively obtain a first diagnosis assistance information related to the presence of macular degeneration of the subject and a second diagnosis assistance information related to the presence of diabetic retinopathy of the subject based on a diagnosis target fundus image.

In addition, unless particularly described otherwise, the diagnosis assistance process described with reference to FIG. 27 may be implemented similar to the diagnosis assistance process described above with reference to FIG. 20.

### 1.4.4.3 Output of Diagnosis Assistance Information

According to an embodiment of the present invention, diagnosis assistance information may be obtained by a parallel diagnosis assistance process. The obtained diagnosis assistance information may be stored in the diagnostic device, server device, and/or client device. The obtained diagnosis assistance information may be transmitted to an external device.

A plurality of diagnosis assistance information may respectively indicate a plurality of labels predicted by a plurality of diagnosis assistance neural network models. The plurality of diagnosis assistance information may respectively correspond to the plurality of labels predicted by the plurality of diagnosis assistance neural network models. Alternatively, diagnosis assistance information may be information determined based on a plurality of labels predicted by a plurality of diagnosis assistance neural network models. The diagnosis assistance information may correspond to the plurality of labels predicted by the plurality of diagnosis assistance neural network models.

In other words, a first diagnosis assistance information may be diagnosis assistance information corresponding to a first label predicted through a first diagnosis assistance neural network model. Alternatively, the first diagnosis assistance information may be diagnosis assistance information determined in consideration of a first label predicted through a first diagnosis assistance neural network model and a second label predicted through a second diagnosis assistance neural network model.

Meanwhile, CAM images obtained from a plurality of diagnosis assistance neural network models may be output. The CAM images may be output when a predetermined condition is satisfied. For example, in any one of the case in which a first diagnosis assistance information indicates that the subject is abnormal in relation to a first characteristic or the case in which a second diagnosis assistance information indicates that the subject is abnormal in relation to a second characteristic, a CAM image obtained from a diagnosis assistance neural network model, from which diagnosis assistance information indicating that the subject is abnormal has been output, may be output.

A plurality of diagnosis assistance information and/or CAM images may be provided to a user. The plurality of diagnosis assistance information or the like may be provided to the user through an output means of the diagnostic device or client device. The diagnosis assistance information may be visually output. This will be described in detail below in Section “5. User interface.”

According to an embodiment of the present invention, diagnosis assistance information corresponding to a diagnosis target image may include level information. The level information may be selected from a plurality of levels. The level information may be determined based on a plurality of diagnostic information and/or findings information obtained through neural network models. The level information may be determined in consideration of suitability information or quality information of a diagnosis target image. The level information may be determined in consideration of a class into which a diagnosis target image is classified by a plurality of neural network models. The level information may be determined in consideration of numerical values output from a plurality of neural network models.

For example, diagnosis assistance information obtained corresponding to a diagnosis target image may include any one level information selected from a first level information and a second level information. When at least one abnormal findings information or abnormal diagnostic information is obtained among of diagnostic information obtained through a plurality of neural network models, the first level information may be selected as the level information. When, of diagnostic information obtained through the neural network models does not include abnormal findings information or abnormal diagnostic information, the second level information may be selected as the level information.

A first level information may be selected as the level information when at least one numerical value among numerical values obtained through a neural network model exceeds a reference numerical value, and a second level information may be selected as the level information when all of the obtained numerical values are less than a reference numerical value. The first of level information may indicate that strong abnormal information is present in a diagnosis target image compared with the second of level information.

A third level information may be selected as the level information when it is determined using image analysis or a neural network model that the quality of a diagnosis target image is a reference quality or lower. Alternatively, diagnosis assistance information may include the third level information together with the first or second level information.

When diagnosis assistance information includes the first level information, a first user guide may be output through an output means. The first user guide may include matters corresponding to at least one of abnormal findings information or abnormal diagnostic information included in diagnosis assistance information. For example, the first user guide may indicate that a more precise test is required for a subject (i.e. patient) corresponding to abnormal information included in diagnosis assistance information. For example, the first user guide may indicate that secondary diagnosis (for example, diagnosis in a separate medical institution or a hospital transfer procedure) is required for the subject. Alternatively, the first user guide may indicate treatment required for the subject. As a specific example, when abnormal information on macular degeneration of the subject is obtained by diagnosis assistance information, the first user guide may include injection prescription and a guide on a hospital transfer procedure (for example, a list of hospitals to which transfer is possible) related to the subject.

When diagnosis assistance information includes the second level information, a second user guide may be output through an output means. The second user guide may include future care plans related to the subject corresponding to the diagnosis assistance information. For example, the second user guide may indicate the time of next visit and the next medical course.

When diagnosis target information includes the third level information, a third user guide may be output through an output means. The third user guide may indicate that a diagnosis target image has to be retaken. The third user guide may include information on the quality of the diagnosis target image. For example, the third user guide may include information on an artifact present in a diagnosis target image (for example, whether the artifact is a bright artifact or a dark artifact, or the degree thereof).

The first to third of level information may be output by an output unit of the client device or diagnostic device. Specifically, the first to third level information may be output through a user interface which will be described below.

### 1.4.5 Embodiment 2—Diagnosis Assistance System

A diagnosis assistance system according to an embodiment of the present invention may include a fundus image obtaining unit, a first processing unit, a second processing unit, a third processing unit, and a diagnostic information output unit.

According to an embodiment of the present invention, the diagnosis assistance system may include a diagnostic device. The diagnostic device may include a fundus image obtaining unit, a first processing unit, a second processing unit, a third processing unit, and/or a diagnostic information output unit. However, the present invention is not limited thereto, and each unit included in the diagnosis assistance system may be disposed at a proper position in a training device, a diagnostic device, a training diagnosis server, and/or a client device. Hereinafter, for convenience of description, the case in which a diagnostic device of a diagnosis assistance system includes a fundus image obtaining unit, a first processing unit, a second processing unit, a third processing unit, and a diagnostic information output unit will be described.

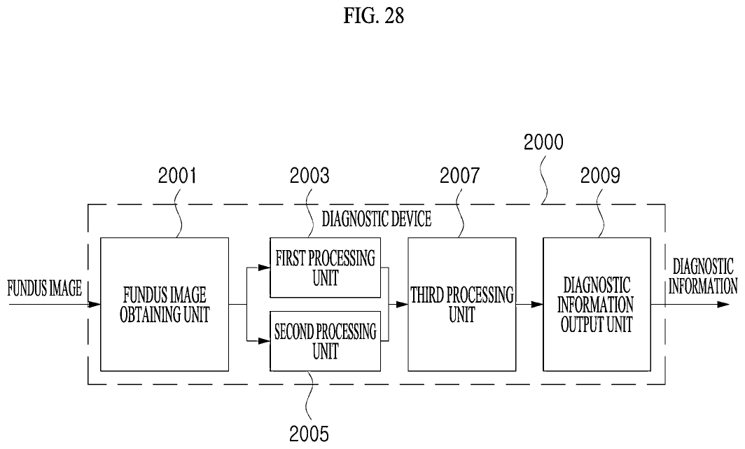

FIG. 28 is a view for describing a diagnosis assistance system according to an embodiment of the present invention. Referring to FIG. 28, a diagnosis assistance system may include a diagnostic device, and the diagnostic device may include a fundus image obtaining unit, a first processing unit, a second processing unit, a third processing unit, and a diagnostic information output unit.

According to an embodiment of the present invention, a diagnosis assistance system that assists diagnosis of a plurality of diseases based on a fundus image may include a fundus image obtaining unit configured to obtain a target fundus image which is a basis for acquiring diagnosis assistance information on a subject, a first processing unit configured to, for the target fundus image, obtain a first result related to a first finding of the subject using a first neural network model, wherein the first neural network model is trained based on a first fundus image set, a second processing unit configured to, for the target fundus image, obtain a second result related to a second finding of the subject using a second neural network model, wherein the second neural network model is trained based on a second fundus image set which is at least partially different from the first fundus image set, a third processing unit configured to determine, based on the first result and the second result, diagnostic information on the subject, and a diagnostic information output unit configured to provide the determined diagnostic information to a user. Here, the first finding and the second finding may be used for diagnosing different diseases.

The first neural network model may be trained to classify an input fundus image as any one of a normal label and an abnormal label in relation to the first finding, and the first processing unit may obtain the first result by classifying the target fundus image as any one of the normal label and the abnormal label using the first neural network model.

The third processing unit may determine whether diagnostic information according to the target fundus image is normal information or abnormal information by taking the first result and the second result into consideration together.

The third processing unit may determine diagnostic information on the subject by assigning priority to the abnormal label so that accuracy of diagnosis is improved.

When the first label is a normal label related to the first finding, and the second label is a normal label related to the second finding, the third processing unit may determine the diagnostic information as normal. When the first label is not the normal label related to the first finding, or the second label is not the normal label related to the second finding, the third processing unit may determine the diagnostic information as abnormal.

The first finding may be related to an eye disease, and the first result may indicate whether the subject is normal in relation to the eye disease. The second finding may be related to a systemic disease, and the second result may indicate whether the subject is normal in relation to the systemic disease.

The first finding may be related to a first eye disease, and the first result may indicate whether the subject is normal in relation to the first eye disease. The second finding may be related to a second eye disease distinguished from the first eye disease, and the second result may indicate whether the subject is normal in relation to the second eye disease.

The first finding may be a finding for diagnosing a first eye disease, and the first result may indicate whether the subject is normal in relation to the first eye disease. The second finding may be a finding distinguished from the first finding for diagnosing the first eye disease, and the second result may indicate whether the subject is normal in relation to a second eye disease.

The first neural network model may include a first sub-neural network model and a second sub-neural network model, and the first result may be determined by taking a first predicted value predicted by the first sub-neural network model and a second predicted value predicted by the second sub-neural network model into consideration together.

The first processing unit may obtain a CAM related to the first label through the first neural network model, and the diagnostic information output unit may output an image of the CAM.

The diagnostic information output unit may output an image of the CAM when the diagnostic information obtained by the third processing unit is abnormal diagnostic information.

The diagnosis assistance system may further include a fourth processing unit configured to obtain quality information on the target fundus image, and the diagnostic information output unit may output the quality information on the target fundus image obtained by the fourth processing unit.

When it is determined in the fourth processing unit that the quality information on the target fundus image is at a predetermined quality level or lower, the diagnostic information output unit may provide information indicating that the quality information on the target fundus image is at the predetermined quality level or lower together with the determined diagnostic information to the user.

### 1.5 User Interface

According to an embodiment of the present invention, the above-described client device or diagnostic device may have a display unit for providing diagnosis assistance information to the user. In this case, the display unit may be provided to facilitate providing of diagnosis assistance information to the user and obtaining of feedback from the user.

As an example of the display unit, a display configured to provide visual information to the user may be provided. In this case, a graphical user interface for visually transferring diagnosis assistance information to the user may be used. For example, in a fundus diagnosis assistance system that obtains diagnosis assistance information based on a fundus image, a graphical user interface for effectively displaying obtained diagnosis assistance information and helping understanding of the user may be provided.

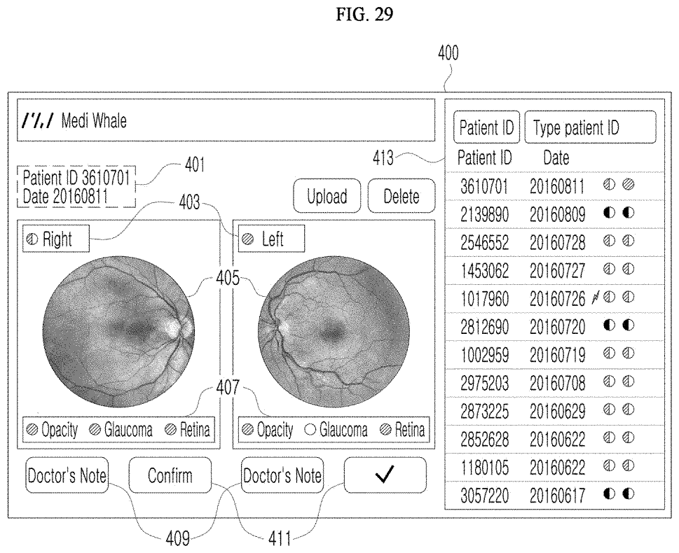

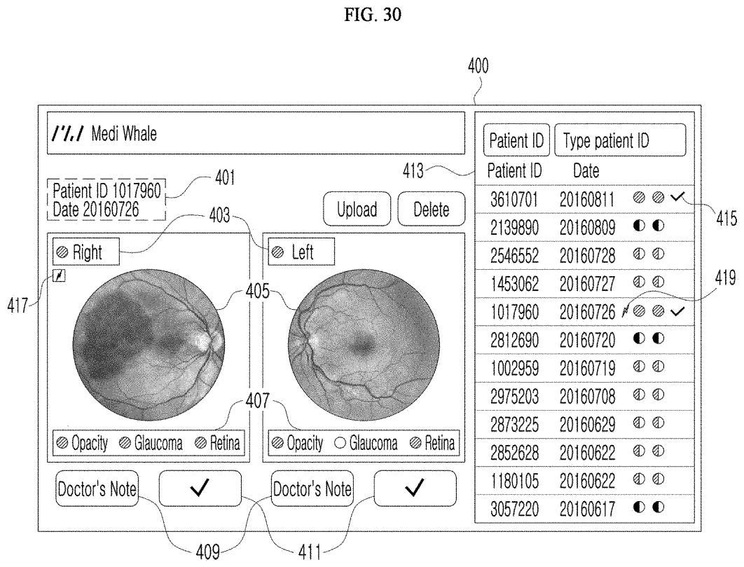

FIGS. 29 and 30 are views for describing a graphical user interface for providing diagnostic information to the user according to some embodiments of the present invention. Hereinafter, some embodiments of a user interface that may be used in a fundus diagnosis assistance system will be described with reference to FIGS. 29 and 30.

Referring to FIG. 29, a user interface according to an embodiment of the present invention may display identification information of a subject corresponding to a diagnosis target fundus image. The user interface may include a target image identification information display unit 401 configured to display identification information of a subject (i.e. patient) and/or imaging information (for example, the data and time of imaging) of a diagnosis target fundus image.

The user interface according to an embodiment of the present invention may include a fundus image display unit 405 configured to display a fundus image of the left eye and a fundus image of the right eye of the same subject. The fundus image display unit 405 may also display a CAM image.

The user interface according to an embodiment of the present invention may include a diagnostic information indicating unit 403 configured to indicate each of the fundus image of the left eye and the fundus image of the right eye as the image of the left eye or right eye and configured to display diagnostic information on each image and a diagnostic information indicator indicating whether the user has confirmed the diagnostic information.

Color of the diagnostic information indicator may be determined in consideration of diagnosis assistance information obtained based on the target fundus image. The diagnostic information indicator may be displayed in a first color or a second color according to the diagnosis assistance information. For example, the diagnostic information indicator may be displayed in red when first to third diagnosis assistance information are obtained from a single target fundus image and when any one of the of diagnosis assistance information includes abnormal information (that is, indicates that there are abnormal findings), and the diagnostic information indicator may be displayed in green when all of the of diagnosis assistance information includes normal information (that is, indicates there are not abnormal findings).

The form of the diagnostic information indicator may be determined according to whether the user has confirmed the diagnostic information. The diagnostic information indicator may be displayed in a first form or a second form according to whether the user has confirmed the diagnostic information. For example, referring to FIG. 29, a diagnostic information indicator corresponding to a target fundus image that has been reviewed by the user may be displayed as a filled circle, and a diagnostic information indicator corresponding to a target fundus image that has not been reviewed by the user yet may be displayed as a half-circle.

The user interface according to an embodiment of the present invention may include a diagnostic information indicating unit 407 configured to indicate diagnosis assistance information. The diagnosis assistance information indicating may be disposed at each of the left eye image and the right eye image. The diagnosis assistance information indicating unit may indicate a plurality of findings information or diagnostic information.

The diagnosis assistance information indicating unit may include at least one diagnosis assistance information indicator. The diagnosis assistance information indicator may indicate corresponding diagnosis assistance information through a color change.

For example, when, in relation to a diagnosis target fundus image, a first diagnosis assistance information indicating the presence of the opacity of crystalline lens is obtained through a first diagnosis assistance neural network model, a second diagnosis assistance information indicating the presence of abnormal findings of diabetic retinopathy is obtained through a second diagnosis assistance neural network model, and a third diagnosis assistance information indicating the presence of abnormal findings of the retina is obtained through a third diagnosis assistance neural network model, the diagnostic information indicating unit may include first to third diagnosis assistance information indicators configured to respectively indicate the first diagnosis assistance information, the second diagnosis assistance information, and the third diagnosis assistance information.

As a more specific example, referring to FIG. 29, when, in relation to the left eye fundus image of the subject, a first diagnosis assistance information indicating that the obtained diagnosis assistance information is abnormal in terms of the opacity of crystalline lens is obtained, a second diagnosis assistance information indicating that the obtained diagnosis assistance information is normal (has no abnormal findings) in terms of diabetic retinopathy is obtained, and a third diagnosis assistance information indicating that the obtained diagnosis assistance information is abnormal (has abnormal findings) in terms of the retina is obtained, the diagnostic information indicating unit 407 may display a first diagnosis assistance information indicator with a first color, a second diagnosis assistance information indicator with a second color, and a third diagnosis assistance information indicator with the first color.

The user interface according to an embodiment of the present invention may obtain a user comment on a diagnosis target fundus image from the user. The user interface may include a user comment object 409 and may display a user input window in response to a user selection on the user comment object. A comment obtained from the user may also be used in updating a diagnosis assistance neural network model. For example, the user input window displayed in response to the user's selection on the user comment object may obtain a user's evaluation on diagnosis assistance information obtained through a neural network, and the obtained user's evaluation may be used in updating a neural network model.

The user interface according to an embodiment of the present invention may include a review indicating object 411 configured to display whether the user has reviewed each diagnosis target fundus image. The review indicating object may receive a user input indicating that the user's reviewing of each diagnosis target image has been completed, and display thereof may be changed from a first state to a second state. Referring to FIGS. 29 and 30, upon receiving a user input, the review indicating object may be changed from a first state in which a review request message is displayed to a second state indicating that the reviewing has been completed.

A diagnosis target fundus image list 413 may be displayed. In the list, identification information of the subject, the data on which the image has been captured, and the indicator 403 of whether the use has reviewed images of the both eyes may be displayed together.

In the diagnosis target fundus image list 413, a review completion indicator 415 indicating that the corresponding diagnosis target fundus image has been reviewed may be displayed. The review completion indicator 415 may be displayed when a user selection has been made for review indicating objects 411 of the both eyes of the corresponding images.

Referring to FIG. 30, the graphical user interface may include a poor quality warning object 417 indicating that there is an abnormality in the quality of a diagnosis target fundus image to the user when it is determined that there is an abnormality in the quality of the diagnosis target fundus image. The poor quality warning object 417 may be displayed when it is determined that the quality of the diagnosis target fundus image from the diagnostic unit is below a quality level at which appropriate diagnosis assistance information may be predicted from a diagnosis assistance neural network model (that is, a reference quality level).

In addition, referring to FIG. 28, the poor quality warning object 419 may also be displayed in the diagnosis target fundus image list 413.

### 2. Plural Information Obtaining Model

Meanwhile, according to the invention described in the present specification, there may be provided a diagnosis assistance neural network model or a diagnosis assistance apparatus which obtains a plurality of diagnosis assistance information.

As will be described in the following embodiments, a neural network model including a common portion and an individual portion is used for obtaining a plurality of diagnosis assistance information, so that accuracy of diagnosis assistance information obtained through the neural network model may be enhanced. As will be described in the following embodiments, when a variety of diagnosis assistance information is desired to be predicted based on an eye image and a plurality of neural network models are individually designed to predict respective pieces of diagnosis assistance information, in the neural network models for predicting different diagnosis assistance information, structures of a few initial layers for extracting abstracted features from an eye image or features extracted from the layers may have similarity. Accordingly, when machine learning is used to obtain a plurality of diagnosis assistance information, a diagnosis assistance neural network model including sharing layers and individual layers may be used to perform a computing process more effectively.

Hereinafter, a diagnosis assistance model for obtaining a plurality of diagnosis assistance information will be described with reference to some embodiments.

### 2.1 Plural Information Obtaining Model Structure

According to an embodiment of the invention described in the present specification, there may be provided a diagnosis assistance neural network model which predicts a plurality of diagnosis assistance information based on the same input. The diagnosis assistance neural network model which predicts a plurality of diagnosis assistance information may have a multi-task model form. The plurality of predicted diagnosis assistance information may be various forms of diagnosis assistance information exemplified in the present specification.

The diagnosis assistance information may be information of the presence of a diagnosis target disease of a subject, a degree of risk, or an index (or a score) related to a disease.

For example, the diagnosis assistance information may be diagnosis assistance information that is used for a diagnosis of an eye disease. The diagnosis assistance information may be diagnosis assistance information related to an eye disease such as glaucoma, cataract, diabetic retinopathy, macular degeneration, bleeding, drusen, abnormal choroid, abnormal retinal blood vessel, nerve fiber layer defect, etc. The diagnosis assistance information may be the presence of a target eye disease regarding a subject, a degree of risk of the target eye disease, numerical information related to the target eye disease.

For example, the diagnosis assistance information may be diagnosis assistance information that is used for a diagnosis of a cardio-cerebrovascular disease. The diagnosis assistance information may be diagnosis assistance information related to a disease related to brain, heart or blood vessel, which includes a coronary artery disease such as heart attack or angina, a coronary heart disease, an ischaemic heart disease, a congestive heart failure, a peripheral vascular disorder, a cardiac arrest, a valvular disease of the heart, a cerebrovascular disease (for example, stroke, cerebral infarction, cerebral hemorrhage, or transient ischemic attack), and a renal vascular disease, etc. The diagnosis assistance information may be the presence of a target cardio-cerebrovascular disease of a subject, a degree of risk of the target cardio-cerebrovascular disease, or numerical information of the target cardio-cerebrovascular disease.

According to an embodiment of the invention described in the present specification, the diagnosis assistance neural network model which predicts a plurality of diagnosis assistance information may have a common portion and an individual portion. The diagnosis assistance neural network model may include a common portion (or a sharing portion) including one or more neural network layers that are used for obtaining a plurality of diagnosis assistance information. Alternatively, first diagnosis assistance neural network model used for predicting first diagnosis assistance information and second diagnosis assistance neural network model used for predicting second diagnosis assistance information may include a common portion (or a sharing portion) including one or more neural network layers which are common to each other. The diagnosis assistance neural network model may include a common portion that is used for predicting one or more pieces of diagnosis assistance information, and an individual portion that does not influence prediction of some diagnosis assistance information.

### 2.1.1 First Type Plural Information Obtaining Model

According to an embodiment, there may be provided a diagnosis assistance neural network model which uses layers distinguished from one another to predict different diagnosis assistance information. For example, according to an embodiment, there may be provided a diagnosis assistance neural network model which has a plurality of individual portions assigned to respective tasks so as to perform several tasks simultaneously.

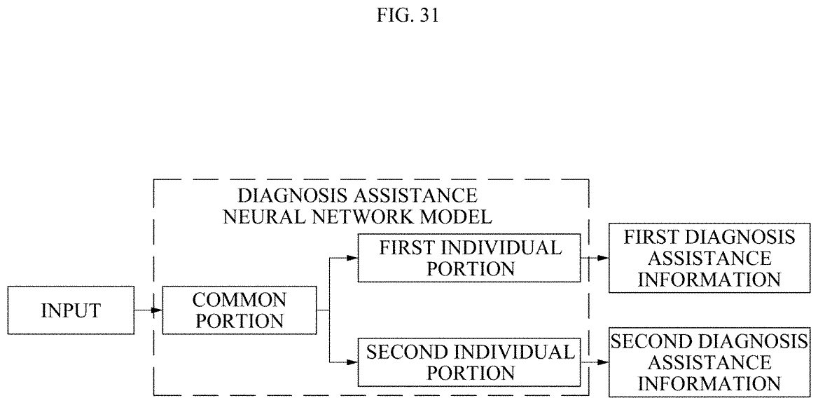

FIG. 31 is a view for describing an embodiment of a diagnosis assistance neural network model described in the present specification. Referring to FIG. 31, the diagnosis assistance neural network model according to an embodiment may include a common portion, first individual portion, and second individual portion.

The common portion may be involved in prediction of a plurality of diagnosis assistance information. The common portion may function as a feature extraction neural network to extract a plurality of features contributing to the prediction of a plurality of diagnosis assistance information. The common portion may obtain a plurality of feature sets based on input data. The common portion may obtain a plurality of feature sets with an eye image obtained from eyes of a subject as an input. The common portion may obtain one or more two-dimensional feature maps.

The common portion may be provided as a neural network including one or more layers. The common portion may include at least one convolution layer or pooling layer. The common portion may include a plurality of convolution layers, and may obtain a plurality of feature values based on an eye image. The common portion may include a fully connected layer.

The common portion may obtain a plurality of feature values or a plurality of feature maps based on an eye image.

The plurality of feature maps may include a plurality of feature maps corresponding to a plurality of elements included in an image. For example, the plurality of feature maps may include first feature map corresponding to a blood vessel element included in an eye image, and second feature map corresponding to a macular element included in the eye image.

The feature set may include a plurality of feature values corresponding to a plurality of elements included in an eye image. For example, the plurality of feature values may include first feature value corresponding to a blood vessel element included in an eye image, and second feature value corresponding to a macular element included in the eye image.

The first individual portion or the second individual portion may obtain first diagnosis assistance information or second diagnosis assistance information, based on one or more feature values or one or more feature maps obtained by the common portion.

The first individual portion may obtain the first diagnosis assistance information based on at least part of the feature set obtained from the common portion. The first individual portion may be trained to obtain the first diagnosis assistance information based on the feature set obtained from the common portion.

The second individual portion may obtain the second diagnosis assistance information based on at least part of the feature set extracted by the common portion. The second individual portion may be trained to obtain the second diagnosis assistance information based on the feature set obtained from the common portion.

The first individual portion or the second individual portion may include one or more neural network layers.

The first individual portion or the second individual portion may include an output layer configured to output the first diagnosis assistance information or the second diagnosis information with the feature set extracted by the common portion as an input value. The first individual portion or the second individual portion may have at least one intermediate layer (or hidden layer) between an input layer and the output layer.

The first individual portion or the second individual portion may include a fully connected layer. The first individual portion or the second individual portion may include at least one convolution layer or pooling layer.

The first individual portion and the second individual portion may have layer structures which are different from each other. For example, the number of layers included in the first individual portion and the number of layers included in the second individual portion may be different. Alternatively, the number of nodes of a layer included in the first individual portion and the number of nodes of a layer included in the second individual portion may be different. The number of nodes of a specific layer included in the first individual portion and the number of nodes of a layer corresponding to the specific layer of the first individual portion among layers included in the second individual portion may be different.

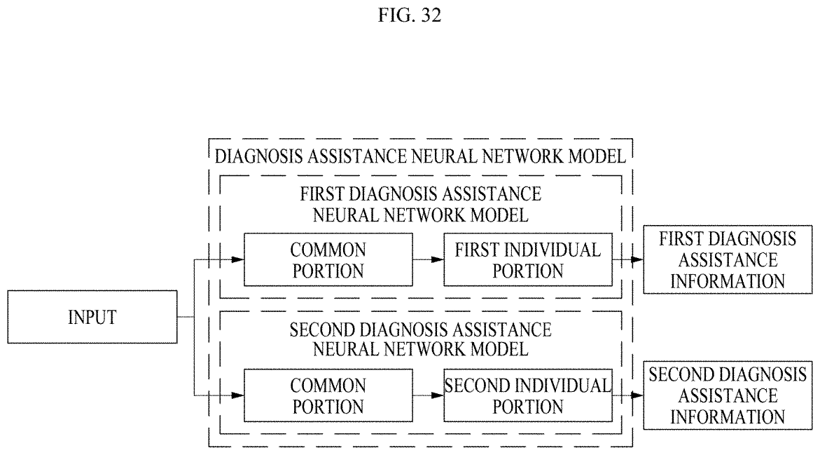

FIG. 32 is a view for describing an embodiment of a diagnosis assistance neural network model described in the present specification. The diagnosis assistance neural network model illustrated in FIG. 31 may be implemented as illustrated in FIG. 32.

Referring to FIG. 32, the diagnosis assistance neural network model may include first diagnosis assistance neural network model configured to obtain first diagnosis assistance information, and second diagnosis assistance neural network model configured to obtain second diagnosis assistance information

The first diagnosis assistance neural network model may obtain the first diagnosis assistance information based on input data (for example, an eye image). The second diagnosis assistance neural network model may obtain the second diagnosis assistance information based on input data.

The first diagnosis assistance neural network model may include a common portion and first individual portion. The second diagnosis assistance neural network model may include a common portion and second individual portion.

The first diagnosis assistance neural network and the second diagnosis assistance neural network may have common portions which are common to each other. The common portion of the first diagnosis assistance neural network and the common portion of the second diagnosis assistance neural network may include at least one convolution neural network, and may extract a plurality of features based on an eye image.

The common portion of the first diagnosis assistance neural network and the common portion of the second diagnosis assistance neural network may be neural network models that have the same layer structure. The common portion of the first diagnosis assistance neural network model and the common portion of the second diagnosis assistance neural network model may have the same layer structure and the same weight. Alternatively, the common portion of the first diagnosis assistance neural network model and the common portion of the second diagnosis assistance neural network model may have the same layer structure, but weights of respective nodes may be different.

The common portion of the second diagnosis assistance neural network may be provided by transferring the common portion of the first diagnosis assistance neural network. The common portion of the second diagnosis assistance neural network may be provided by fine-tuning transferring the common portion of the first diagnosis assistance neural network. The common portion of the second diagnosis assistance neural network may use the common portion of the first diagnosis assistance neural network as a pre-trained model, and may have the same weight as that of the common portion of the first diagnosis assistance neural network. The common portion of the second diagnosis assistance neural network may be provided by reusing some layers of the common portion of the first diagnosis assistance neural network, or by performing domain adaption.

The first individual portion of the first diagnosis assistance neural network and the second individual portion of the second diagnosis assistance neural network may have different layer structures. The first individual portion and the second individual portion may be different in terms of the number of layers, the number of nodes, or the weight value, etc.

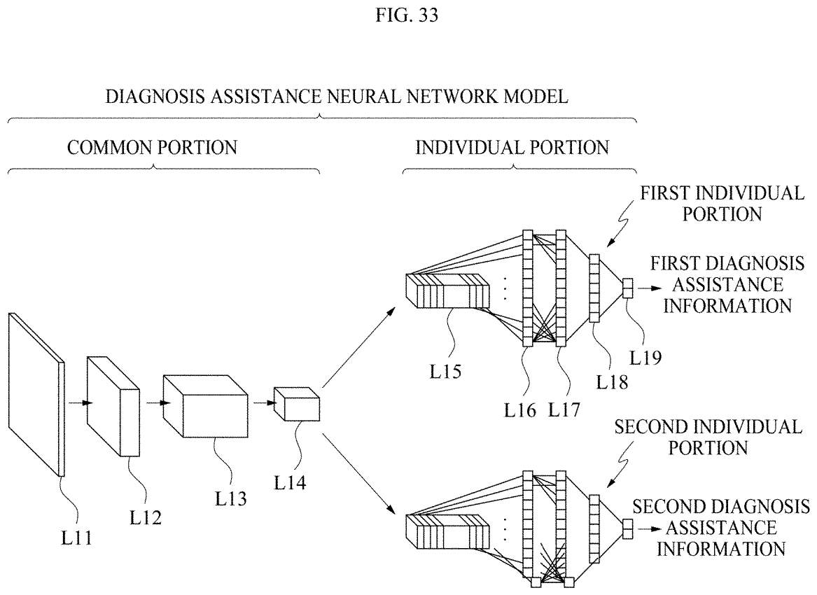

FIG. 33 is a view for describing an embodiment of a diagnosis assistance neural network model described in the present specification.

Referring to FIG. 33, the diagnosis assistance neural network model according to an embodiment may include a common portion including a plurality of convolution neural network layers, and an individual portion including a fully connected layer.

The common portion may include an input layer L11. The common portion may include first convolution layer L12 which is obtained by applying first convolution filter to the input layer L1. The common portion may include second convolution layer L3 which is obtained by applying second convolution filter to the first convolution layer L2. The common portion may include third convolution layer L14 which is obtained by applying third convolution filter to the second convolution layer L13. The first to third convolution layers may be obtained by applying a convolution filter and pooling (for example, max pooling) to a previous layer. Although FIG. 33 illustrates three convolution layers, the diagnosis assistance neural network model may include more or fewer convolution layers.

Referring to FIG. 33, the individual portion may include first individual portion configured to obtain first diagnosis assistance information, and second individual portion configured to obtain second diagnosis assistance information. The first individual portion may include first individual layer L15 which is obtained by applying first individual convolution filter, based on the third convolution layer L14. The first individual portion may include second individual layer L16 which is obtained by flattening the first individual layer L15. The first individual portion may include third individual layer L17 which is fully connected with the second individual layer L16 and has the same number of nodes as those of the second individual layer L16. The first individual portion may include fourth individual layer L18 which is fully connected with the third individual layer L17 and has a smaller number of nodes than the third individual layer L17. The first individual portion may include an output layer L19 which is fully connected with the fourth individual layer L18.

Referring to FIG. 33, the second individual portion may be implemented in a form similar to the first individual portion. Although the case in which the first individual portion and the second individual portion have a similar layer structure has been described with reference to FIG. 33, the respective individual portions may be designed with different structures. The second individual portion may be implemented to have the different number of layers, the different number of nodes, a different weight or a different output form from those of the first individual portion.

The individual portion included in the diagnosis assistance neural network model may include more or fewer convolution layers or fully-connected layers than those illustrated in FIG. 33. The individual portion may include an output layer which has more or fewer nodes than those illustrated in FIG. 33.

According to an embodiment, the diagnosis assistance neural network model may be provided as one neural network model including a common portion, first individual portion, and second individual portion. Alternatively, the diagnosis assistance neural network model may be provided with first diagnosis assistance neural network model including a common portion and first individual portion, and second diagnosis assistance neural network model including a common portion and second individual portion.

Although the case in which a common portion includes a convolution neural network layer and an individual portion includes a fully connected layer has been described with reference to FIG. 33, the diagnosis assistance neural network model described in the present specification is not limited thereto. For example, a common portion may include a fully connected layer or an individual portion may include a convolution neural network.

### 2.1.2 Second Type Plural Information Obtaining Model

According to an embodiment, an individual portion may be positioned before a common portion. When use of different features of an eye image is required for prediction of different diagnosis assistance information, the individual portion positioned before the common portion may be used. Alternatively, when extraction of different features of an eye image is required, a diagnosis assistance neural network model in which a plurality of individual portions are positioned before a common portion may be used.

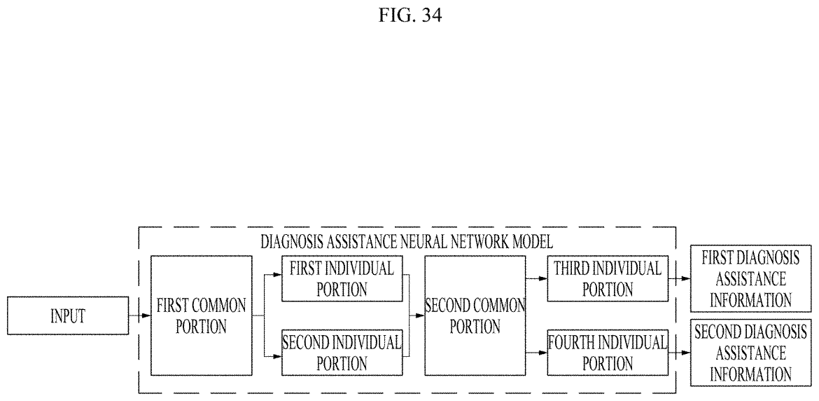

FIG. 34 is a view for describing another embodiment of a diagnosis assistance neural network model described in the present specification. Referring to FIG. 34, the diagnosis assistance neural network model according to an embodiment may include a common portion that is positioned between individual portions.

Referring to FIG. 34, the diagnosis assistance neural network model may include first common portion configured to obtain first feature set with an eye image as input data, first individual portion configured to obtain second feature set based on the first feature set, second individual portion configured to obtain third feature set based on the first feature set, second common portion configured to obtain fourth feature set based on the second feature set and/or the third feature set, third individual portion configured to obtain first diagnosis assistance information based on the fourth feature set, and fourth individual portion configured to obtain second diagnosis assistance information based on the fourth feature set. The first to fourth feature sets may include at least one 2D feature map or at least one feature value. The diagnosis assistance neural network model according to an embodiment may include a structure in which the common portion and the individual portion illustrated in FIG. 34 are repeated. According to another embodiment, the first common portion may be omitted.

The first common portion may obtain the first feature set including at least one feature based on the eye image. The first common portion may include at least one convolution neural network layer, and the first feature set may include at least one feature map. The first feature set may be a feature set that is related to the first diagnosis assistance information.

The first individual portion and the second individual portion may include at least one convolution neural network layer. The first individual portion and the second individual portion may obtain different feature sets with the same eye image as an input.

The first individual portion and the second individual portion may have different layer structures. The first individual portion and the second individual portion may have the different numbers of nodes, the different number of layers, different weights or different convolution filters.

The first individual portion and the second individual portion may extract different features based on input data that is at least partially common. For example, the first individual portion may obtain the second feature set based on the first feature set obtained by the first common portion, and the second individual portion may obtain the third feature set based on the first feature set. The second feature set and the third feature set may be the same kinds of feature sets.

According to an embodiment, the first common portion may be omitted and the first individual portion and the second individual portion may extract features based on different input data. The first individual portion and the second individual portion may extract features based on eye images with respect to which different pre-processing is performed. For example, the first individual portion may extract second feature based on the eye image, and the second individual portion may extract third feature based on a blood vessel-emphasized eye image. According to an embodiment, the first common portion may be omitted, and the first individual portion may extract the second feature based on the eye image, and the second individual portion may extract the third feature based on the blood vessel-emphasized eye image.

The second feature set may be a feature set that is related to the first diagnosis assistance information. The third feature set may be a feature set that is related to the second diagnosis assistance information.

The second common portion may extract the fourth feature set based on the first feature set and/or the second feature set. The fourth feature set may be a feature set that is related to the first diagnosis assistance information and/or the second diagnosis assistance information.

The third individual portion and the fourth individual portion may obtain diagnosis assistance information based at least in part on the fourth feature set. The third individual portion and the fourth individual portion may have different layer structures. The third individual portion and the fourth individual portion may have the different number of nodes, the different number of layers, different weights or different convolution filters.

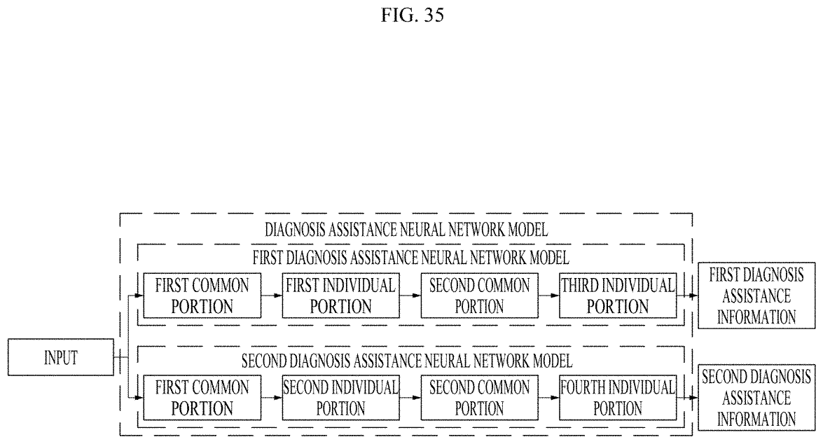

FIG. 35 is a view for describing an embodiment of a diagnosis assistance neural network model described in the present specification. The diagnosis assistance neural network model illustrated in FIG. 34 may be implemented as illustrated in FIG. 35.

Referring to FIG. 35, the diagnosis assistance neural network model may include first diagnosis assistance neural network model configured to obtain first diagnosis assistance information and second diagnosis assistance neural network model configured to obtain second diagnosis assistance information.

The first diagnosis assistance neural network model may include first common portion, first individual portion, second individual portion, and third individual portion. The second diagnosis assistance neural network model may include second individual portion, a common portion, and fourth individual portion. The contents described above in relation to FIG. 34 may be similarly applied to each individual portion and each common portion.

The respective common portions of the first diagnosis assistance neural network model and the respective common portions of the second diagnosis assistance neural network model may have the same layer structure. The respective common portions of the first diagnosis assistance neural network model and the respective common portions of the second diagnosis assistance neural network model may include identical layers that are trained together with the same layer structure. Alternatively, the common portion of the first diagnosis assistance neural network model and the common portion of the second diagnosis assistance neural network model may have the same layer structure, but may be separately trained and provided.

The common portion of the second diagnosis assistance neural network may be provided by transferring the common portion of the first diagnosis assistance neural network. The common portion of the second diagnosis assistance neural network may be provided by fine tuning the common portion of the first diagnosis assistance neural network, using as a pre-trained model, reusing some layers, or performing domain adaption.

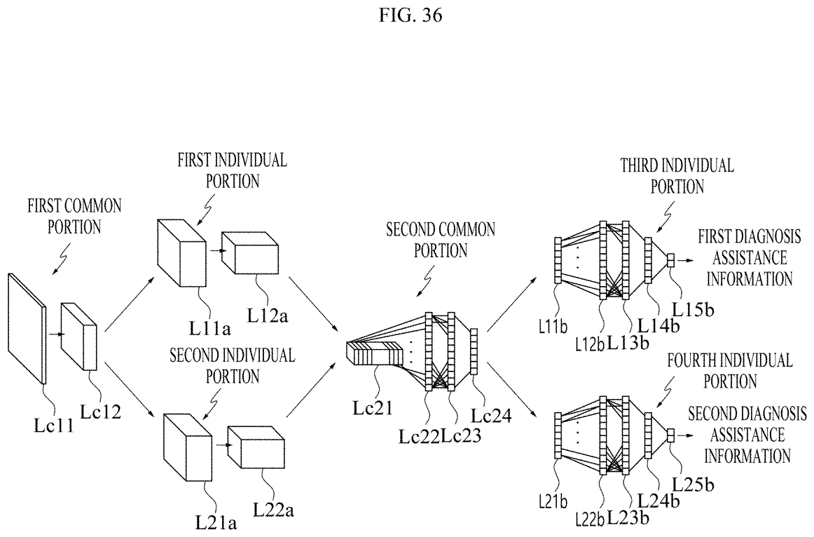

FIG. 36 is a view for describing a more specific embodiment of a diagnosis assistance neural network model described in the present specification. The diagnosis assistance neural network model illustrated in FIG. 34 may be implemented as illustrated in FIG. 36. The diagnosis assistance neural network model described in FIG. 36 may be implemented similarly to that of FIG. 34 unless particularly described otherwise.

Referring to FIG. 36, the diagnosis assistance neural network model may include first common portion, first individual portion, second individual portion, second common portion, third individual portion, and fourth individual portion. The first individual portion and the second individual portion may be positioned after the first common portion and before the second common portion, and the third individual portion and the fourth individual portion may be positioned after the second common portion. Referring to FIG. 36, first feature set, second feature set, and third feature set may be a plurality of feature maps that are obtained through convolution filters, respectively.

Referring to FIG. 36, the first common portion may include an Lc11 layer which is an input layer, and an Lc12 layer which is obtained by applying first convolution filter to the Lc11 layer. The first individual portion may include a 11a layer L11a, a 12a layer L12a which is obtained by applying second convolution filter to the 11a layer, and a 13a layer L13a which is obtained by applying third convolution layer to the 12a layer. Referring to FIG. 36, the second individual portion may include a 21a layer L21a, a 22a layer L22a which is obtained by applying fourth convolution filter to the 21a layer, and a 23a layer L23a which is obtained by applying fifth convolution filter to the 22a layer. Each individual portion may include more or fewer convolution layers than those illustrated in FIG. 34.

Referring to FIG. 34, the second common portion may obtain fourth feature set with first feature map set obtained by the first individual portion and/or second feature map set obtained by the second individual portion as an input. Referring to the example of FIG. 34, the fourth feature set may be a plurality of feature values.

Referring to FIG. 36, the common portion may include: first common layer Lc1 which is obtained based on the 13a layer L13a and the 23a layer L23a and includes a plurality of feature maps; second common layer Lc2 which is obtained based on the first common layer Lc1 and includes a plurality of feature values; third common layer Lc3 which is a fully connected layer fully connected with respective nodes of the second common layer Lc2; and fourth common layer Lc4 which is fully connected with respective nodes of the third common layer Lc3. The common portion may include more or fewer layers or node than those illustrated in FIG. 34.

The third individual portion may obtain first diagnosis assistance information based on the third feature set. The fourth individual portion may obtain second diagnosis assistance information, which is different from the first diagnosis assistance information, based on the third feature set. Referring to FIG. 36, the third individual portion and the fourth individual portion may obtain diagnosis assistance information through a neural network including a plurality of fully connected layer, based on an input including a plurality of feature values obtained through the common portion.

Referring to FIG. 36, the third individual portion may include: a 11b layer L11b which is obtained based on the fourth common layer Lc4; a 12b layer L12b which is a hidden layer fully connected with the 11b layer L11b; a 13b layer L13b which is a hidden layer fully connected with the 12b layer L12b; a 14b layer L14b which is a hidden layer fully connected with the 13b layer L13b, and a 15b layer L15b which is an output layer connected with the 14b layer L14b. Referring to FIG. 36, the fourth individual portion may include: a 21b layer L21b which is obtained based on the fourth common layer Lc4; a 22b layer L22b which is a hidden layer fully connected with the 21b layer L21b; a 23b layer L23b which is a hidden layer fully connected with the 22b layer L22b; a 24b layer L24b which is a hidden layer fully connected with the 23b layer L23b; and a 25b layer L25b which is an output layer connected with the 24b layer L24b. Each individual portion may include more or fewer convolution layers than those illustrated in FIG. 36.

### 2.1.3 Third Type Plural Information Obtaining Model

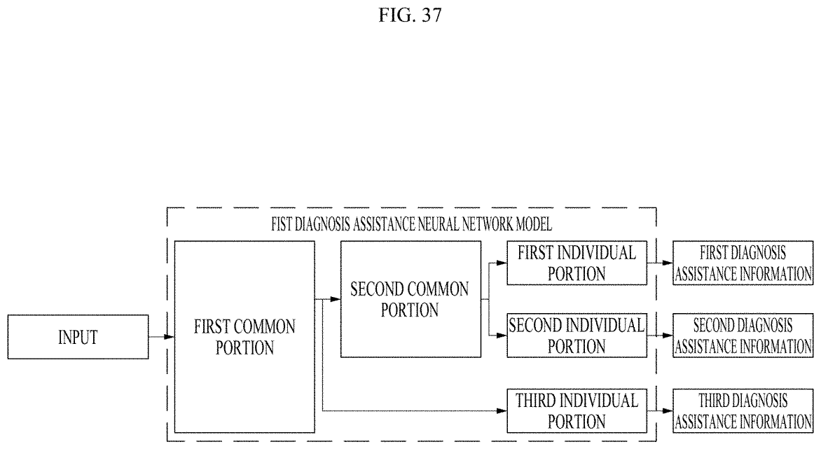

FIG. 37 is a view for describing a diagnosis assistance neural network model according to an embodiment of the invention described in the present specification. Referring to FIG. 37, the diagnosis assistance neural network model according to an embodiment may obtain first diagnosis assistance information, second diagnosis assistance information, and third diagnosis assistance information.

Referring to FIG. 37, the diagnosis assistance neural network model according to an embodiment may include: first common portion configured to obtain first feature set based on an eye image; second common portion configured to obtain second feature set based at least in part on the first feature set; first individual portion configured to obtain first diagnosis assistance information based at least in part on the second feature set; second individual portion configured to obtain second diagnosis assistance information based at least in part on the second feature set; and third individual portion configured to obtain third diagnosis assistance information based at least in part on the first feature set.

The first common portion may obtain an eye image and may obtain the first feature set through at least one convolution neural network layer. The first feature set may include a plurality of feature maps or feature values. The first common portion may include a plurality of layers associated with the first, second, and third diagnosis assistance information.

The second common portion may obtain the second feature set through at least one convolution neural network layer or at least one fully connected layer, based on at least part of the first feature set. For example, when the first feature set includes a plurality of feature maps, the second common portion may include at least one convolution neural network and may obtain the second feature set. For example, when the first feature set includes a plurality of feature values, the second common portion may include at least one fully connected layer and may obtain the second feature set. The second feature set may include a plurality of feature maps or a plurality of feature values. The second common portion may include a plurality of layers associated with the first and second diagnosis assistance information.

The first common portion may obtain the first feature set which includes a plurality of feature maps or a plurality of feature values, based on the eye image. The first feature set may include a plurality of feature maps corresponding to a plurality of elements included in the eye image. For example, the first feature set may include first feature map corresponding to a blood vessel element included in the eye image, and second feature map corresponding to a macular element included in the eye image. The first feature set may include a plurality of feature values corresponding to a plurality of elements included in the eye image. For example, the first feature set may include first feature value corresponding to a blood vessel element included in the eye image, and second feature value corresponding to a macular element included in the eye image.

The second common portion may obtain the second feature set which includes a plurality of feature maps or a plurality of feature values, based on the eye image. The second feature set may include a plurality of feature maps corresponding to a plurality of elements included in the eye image. Alternatively, the second feature set may include a feature map or a feature value that corresponds to a more specified element than the element corresponding to the feature map or feature value included in the first feature set. For example, the second feature set may include first feature map (or first feature value) corresponding to drusen included in the eye image, and second feature map (or second feature value) corresponding to macular degeneration included in the eye image.

The first individual portion may obtain the first diagnosis assistance information through at least one convolution neural network layer or at least one fully connected layer, based on at least part of the second feature set. For example, when the second feature set includes a plurality of feature maps, the first individual portion may obtain the first diagnosis assistance information through at least one convolution neural network layer and at least one fully connected layer. When the second feature set includes a plurality of feature values, the first individual portion may obtain the first diagnosis assistance information through at least one fully connected layer.

The second individual portion may obtain the second diagnosis assistance information through at least one convolution neural network layer or at least one fully connected layer, based on at least part of the second feature set. For example, when the second feature set includes a plurality of feature maps, the second individual portion may obtain the second diagnosis assistance information through at least one convolution neural network layer and at least one fully connected layer. When the second feature set includes a plurality of feature values, the second individual portion may obtain the second diagnosis assistance information through at least one fully connected layer.

The second diagnosis assistance information may be diagnosis assistance information that is different from the first diagnosis assistance information. The second diagnosis assistance information may be diagnosis assistance information of a different format regarding the same disease as the first diagnosis assistance information. For example, the first diagnosis assistance information may be information regarding the presence of a specific eye disease, and the second diagnosis assistance information may be score information regarding the specific eye disease. In addition, for example, the first diagnosis assistance information may be information regarding the presence of a specific cerebral cardiovascular disease, and the second diagnosis assistance information may be a score related to the specific cerebral cardiovascular disease (for example, a coronary artery calcium score).

The third individual portion may obtain the third diagnosis assistance information through at least one convolution neural network layer and/or at least one fully connected layer, based at least in part on the first feature set. For example, the third individual portion may obtain the third diagnosis assistance information through at least one convolution neural network layer when the first feature set includes a plurality of feature maps, and may obtain the third diagnosis assistance information through at least one fully connected layer when the first feature set includes a plurality of feature values.

The third diagnosis assistance information may be diagnosis assistance information that is different from the first diagnosis assistance information and the second diagnosis assistance information. The third diagnosis assistance information may be diagnosis assistance information that belongs to a different group from the first diagnosis assistance information and the second diagnosis assistance information. For example, the first and second diagnosis assistance information may be diagnosis assistance information related to an eye disease, and the third diagnosis assistance information may be diagnosis assistance information related to a non-eye disease (for example, a systemic disease or a cerebral cardiovascular disease).

When pieces of diagnosis assistance information correlate with each other, the diagnosis assistance neural network model may be designed to have more common portions of a layer associated with each diagnosis assistance information. In order words, the first diagnosis assistance information and the second diagnosis assistance information may correlate with each other, and the third diagnosis assistance information may have a low correlation with the first or second diagnosis assistance information. In this case, a common portion of layers associated with the first diagnosis assistance information and layers associated with the second diagnosis assistance information may include more layers than a common portion of layers associated with the first or second diagnosis assistance information and layers associated with the third diagnosis assistance information. Referring to FIG. 37, a common portion associated with all of the first, second, and third diagnosis assistance information may be the first common portion, and a common portion associated with the first and second diagnosis assistance information correlating with each other may be the first common portion and the second common portion.

Although the embodiment in which the first to third diagnosis assistance information is obtained by the diagnosis assistance neural network model has been described in FIG. 37, according to the invention described in the present specification, there may be provided a diagnosis assistance neural network model which obtains more pieces of diagnosis assistance information than those illustrated in FIG. 37.

According to an embodiment, a plurality of diagnosis assistance information predicted by a diagnosis assistance neural network model may be grouped according to their correlation. For example, the plurality of diagnosis assistance information may include first group and second group. The first group may include first diagnosis assistance information and second diagnosis assistance information, and the second group may include third diagnosis assistance information and fourth diagnosis assistance information.

The pieces of diagnosis assistance information included in each group may have a correlation. For example, the first diagnosis assistance information and the second diagnosis assistance information included in the first group may be diagnosis assistance information related to first disease, and the third diagnosis assistance information and the fourth diagnosis assistance information included in the second group may be diagnosis assistance information related to second disease which is different from the first disease. For example, the diagnosis assistance information included in the first group may be diagnosis assistance information related to an eye disease, and the diagnosis assistance information included in the second group may be diagnosis assistance information related to a systemic disease (or a cerebral cardiovascular disease).

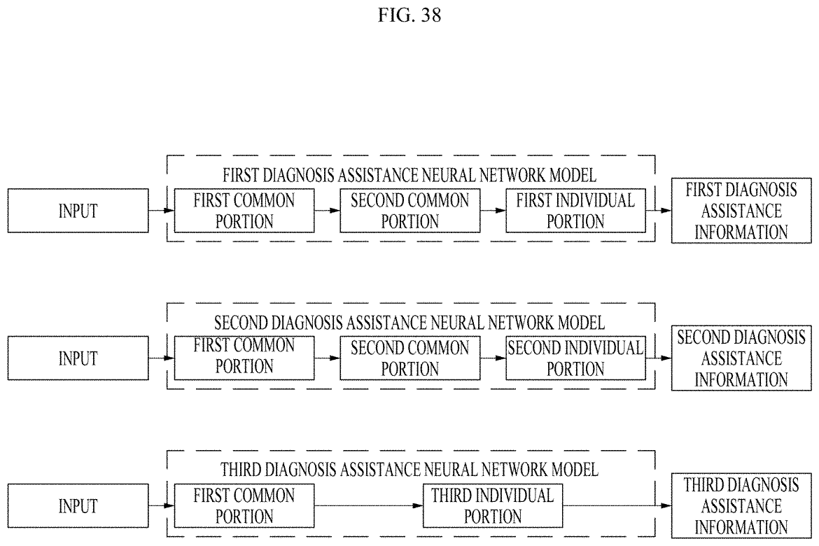

FIG. 38 is a view for describing a diagnosis assistance neural network model according to an embodiment of the invention described in the present specification. According to an embodiment, the diagnosis assistance neural network model illustrated in FIG. 37 may be implemented as illustrated in FIG. 38.

Referring to FIG. 38, according to an embodiment of the invention described in the present specification, there may be provided first diagnosis assistance neural network model configured to obtain first diagnosis assistance information, second diagnosis assistance neural network model configured to obtain second diagnosis assistance information, and third diagnosis assistance neural network model configured to obtain third diagnosis assistance information.

The first diagnosis assistance neural network model may include first common portion configured to obtain an eye image and to extract first feature set, second common portion configured to extract second feature set based on the first feature set, and first individual portion configured to extract the first diagnosis assistance information based on the second feature set.

The second diagnosis assistance neural network model may include first common portion configured to obtain an eye image and to extract first feature set, second common portion configured to extract second feature set based on the first feature set, and second individual portion configured to extract the second diagnosis assistance information based on the second feature set.

The third diagnosis assistance neural network model may include first common portion configured to obtain an eye image and to obtain first feature set, and third individual portion configured to obtain the third diagnosis assistance information based on the first feature set.

The first common portions of the first to third diagnosis assistance neural network models may include the same layers. The respective first common portions may have the same layer structure and/or weight values. The respective first common portions may have the same layer structure and may be separately trained and provided. The second common portions of the first and second diagnosis assistance neural network models may include the same layers. The respective second common portions may have the same layer structure and/or weight values.

The first common portions of the second and third diagnosis assistance neural network models may be provided by transferring the first common portion of the first diagnosis assistance neural network (or the third diagnosis assistance neural network). The first common portions of the second and third diagnosis assistance neural network models may be provided by fine-tuning the first common portion of the first diagnosis assistance neural network (or the third diagnosis assistance neural network), using as a pre-trained model, reusing some layers, or performing domain adaption.

The second common portion of the second diagnosis assistance neural network model may be provided by transferring the second common portion of the first diagnosis assistance neural network. The second common portion of the second diagnosis assistance neural network model may be provided by fine-tuning the second common portion of the first diagnosis assistance neural network, using as a pre-trained model, reusing some layers, or performing domain adaptation.

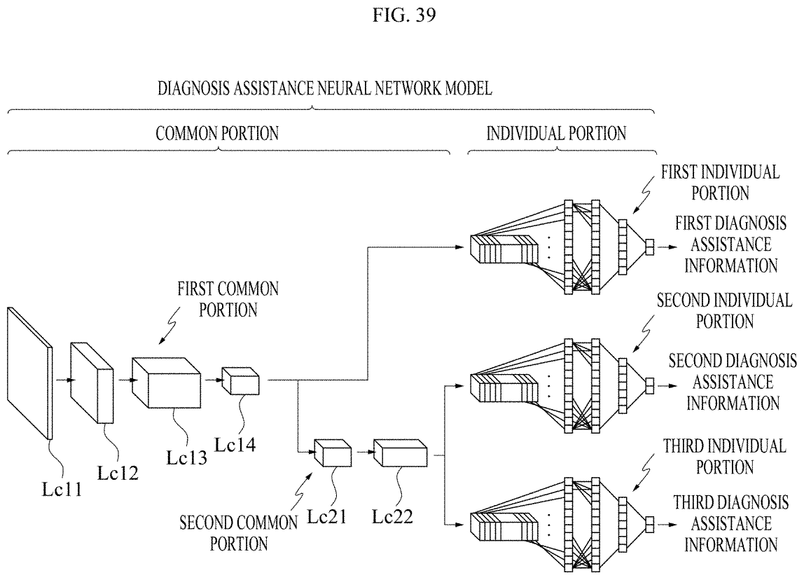

FIG. 39 is a view for describing a more specific example of a diagnosis assistance neural network model according to an embodiment described in the present specification. Contents illustrated in FIGS. 37 to 38 may be similarly applied to the diagnosis assistance neural network model illustrated in FIG. 39 unless particularly described otherwise. Hereinafter, descriptions will be made with reference to second common portion.

Referring to FIG. 39, the diagnosis assistance neural network model according to an embodiment may include first common portion, second common portion, first individual portion, second individual portion, and third individual portion.

The first common portion may include: a 11 common layer Lc11 which is an input layer according to an eye image; a 12 common layer Lc12 which is obtained by applying first convolution filter to the 11 common layer Lc11; a 13 common layer Lc13 which is obtained by applying second convolution filter to the 12 common layer Lc12; and a 14 common layer Lc14 which is obtained by applying third convolution filter to the 12 common layer Lc12. The 14 common layer Lc14 may provide first feature set.

The second common portion may include a 21 common layer Lc21 configured to obtain a feature set (a plurality of feature maps in the example of FIG. 39) from the 14 common layer Lc14, and a 22 common layer Lc22 which is obtained based on the 21 common layer Lc21. The 22 common layer Lc22 may provide second feature set.

The first individual portion may obtain first diagnosis assistance information based on the first feature set. The first individual portion may include a flatten layer and a plurality of fully connected layers configured to obtain a plurality of feature values based on the first feature set, and may obtain the first diagnosis assistance information. The second individual portion may obtain second diagnosis assistance information based on the second feature set. The second individual portion may include a flatten layer and a plurality of fully connected layers configured to obtain a plurality of feature values based on the second feature set, and may obtain the second diagnosis assistance information. The second individual portion may obtain the second diagnosis assistance information based on the second feature set. The third individual portion may include a flatten layer and a plurality of fully connected layers configured to obtain a plurality of feature values based on the second feature set, and may obtain third diagnosis assistance information. Contents described above in relation with FIG. 39 may be similarly applied to a configuration of each individual portion.

Each common portion and each individual portion may include more or fewer layers or nodes than those illustrated in FIG. 39. Layers constituting each common portion or individual portion may be included in other portions.

In addition to the forms illustrated in FIGS. 31 to 39, various forms of diagnosis assistance neural network models which predict a plurality of diagnosis assistance information may be implemented. For example, according to the invention described in the present specification, a diagnosis assistance neural network model having a plurality of common portions, an individual portion positioned before a common portion and an individual portion positioned after a common portion may be used.

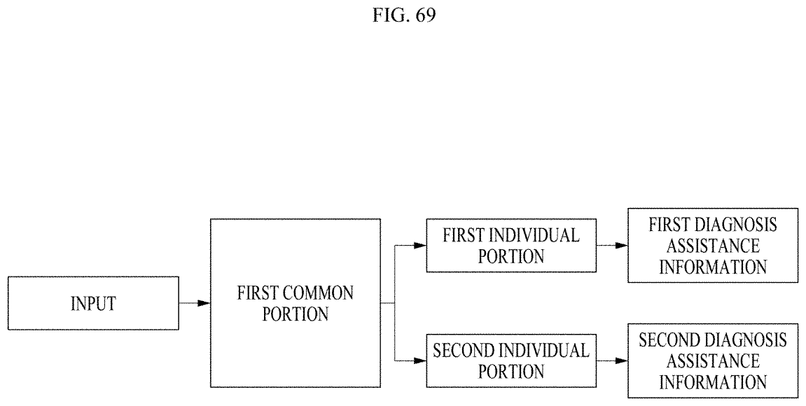

FIG. 69 is a view for describing a diagnosis assistance neural network device according to an embodiment. Referring to FIG. 69, the diagnosis assistance neural network device may obtain input data, and may obtain first diagnosis assistance information and second diagnosis assistance information through first common portion, first individual portion and second individual portion.

According to an embodiment, there may be provided a diagnosis assistance apparatus which uses a neural network model including at least one neural network layer and obtains diagnosis assistance information based on an eye image, wherein the diagnosis assistance apparatus includes: an eye image obtaining unit configured to obtain a target eye image which is obtained from eyes of a subject, and a processing unit using a neural network model trained to obtain diagnosis assistance information based on the eye image and configured to obtain the diagnosis assistance information based on the target eye image.

The neural network model may include first diagnosis assistance neural network model configured to obtain first diagnosis assistance information based on the target eye image, and second diagnosis assistance neural network model configured to obtain second diagnosis assistance information which is different from the first diagnosis assistance information, based on the target eye image.

The first diagnosis assistance neural network model may include first common portion configured to obtain first feature set based on the target eye image, and first individual portion configured to obtain the first diagnosis assistance information based on the first feature set, and the second diagnosis assistance neural network model may include the first common portion configured to obtain the first feature set based on the target eye image; and second individual portion configured to obtain second diagnosis assistance information based on the first feature set.

The first individual portion may be trained based on first training data, and the first individual portion may be trained based on second training data which is different from the first training data at least in part.

The first individual portion may be trained based on the first training data which includes an eye image and first label. The second individual portion may be trained based on the second training data which includes an eye image and second label. The first label and the second label may include information regarding different diseases. For example, the first label may indicate the presence of a disease regarding first eye disease of a subject, and the second label may indicate the presence of a disease regarding second eye disease of the subject. Alternatively, the first label may indicate the presence of the first eye disease of the subject, and the second label may indicate the presence of first cerebral cardiovascular disease of the subject.

According to an embodiment, the first diagnosis assistance information may include at least one piece of diagnosis assistance information related to the first eye disease, and the second diagnosis assistance information may include at least one piece of diagnosis assistance information related to the second eye disease which is different from the first eye disease.

The first feature set may include a plurality of feature values associated with the first diagnosis assistance information and the second diagnosis assistance information. The first individual portion may obtain the first diagnosis assistance information based on at least one feature value included in the first feature set. The second individual portion may obtain the second diagnosis assistance information based on at least one feature value included in the first feature set.

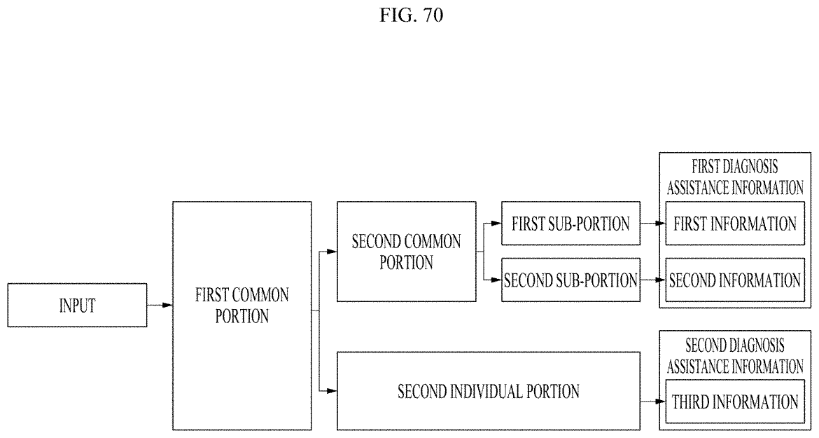

FIG. 70 is a view for describing a diagnosis assistance apparatus according to an embodiment. Referring to FIG. 70, the diagnosis assistance apparatus according to an embodiment may obtain first diagnosis assistance information and second diagnosis assistance information by using a neural network model which includes first common portion, second common portion, first sub-portion, second sub-portion, and second individual portion.

According to an embodiment, the first diagnosis assistance information may include first information and second information

First individual portion may include: the second common portion configured to obtain second feature set which includes a plurality of feature values associated with the first information and the second information, based at least in part on first feature set; the first sub-portion configured to obtain the first information based at least in part on the second feature set; and the second sub-portion configured to obtain the second information based at least in part on the second feature set.

The first information and the second information may be diagnosis assistance information related to a disease related to first part of a human body, and the second diagnosis assistance information may be diagnosis assistance information related to a disease of second part of the human body. The second part may be different from the first part.

For example, the first information may be diagnosis assistance information indicating the presence of glaucoma of eyes of a subject, the second information may be diagnosis assistance information indicating the presence of diabetic retinopathy of eyes of the subject, and the second diagnosis assistance information may be diagnosis assistance information indicating a degree of calcification of a coronary artery of the subject.

The first diagnosis assistance information may include at least one piece of diagnosis assistance information related to an eye disease, and the second diagnosis assistance information may include at least one piece of diagnosis assistance information related to a cerebral cardiovascular disease.

The first diagnosis assistance information may include at least one piece of diagnosis assistance information related first eye disease, and the second diagnosis assistance information may include at least one piece of diagnosis assistance information related to second eye disease which is different from the first eye disease.

The first diagnosis assistance information may include diagnosis assistance information related to glaucoma, and the second diagnosis assistance information may include diagnosis assistance information related to a coronary artery disease.

The processing unit may further include a pre-processing unit configured to perform pre-processing for emphasizing a blood vessel included in a target eye image and to obtain a blood vessel-emphasized eye image. The first common portion may obtain the first feature set based on the blood vessel-emphasized eye image.

According to an embodiment, the first feature set and/or the second feature set may include at least one feature map. For example, the first feature set may include at least one feature map, and the second feature set may include at least one feature value. The first feature set and/or the second feature set may include a feature map and/or a feature value corresponding an element included in the eye image.

### 2.2. Plural Information Prediction Model Training

According to an embodiment of the invention described in the present specification, there may be provided a method for training a neural network model for predicting a plurality of diagnosis assistance information described above, based on eye image data.

Hereinafter, contents of the above-described training process may be similarly applied unless particularly described otherwise. For example, in a training process of a neural network model for predicting a plurality of diagnosis assistance information, which will be described below, an image that is processed according to a data processing process such as image resizing, pre-processing, augmentation, etc. described above may be used as training data. In addition, in the training process of the neural network model for predicting the plurality of diagnosis assistance information, which will be described below, contents of the above-described training process, for example, a general training process of a model, a test, an ensemble, etc. may be applied correspondingly. The training of the diagnosis assistance neural network, which will be described below, may be performed through a framework such as Theano, Keras, Caffe, Torch, Microsoft Cognitive Toolkit (CNTK), Apache MXNet, etc.

Hereinafter, a training method of a neural network model for predicting a plurality of diagnosis assistance information will be described with reference to some embodiments.

### 2.2.1 First Type Model Training

According to an embodiment of the invention described in the present specification, a diagnosis assistance neural network model for predicting a plurality of diagnosis assistance information may be trained by using eye image training data including a plurality of labels.

The diagnosis assistance neural network model for predicting a plurality of diagnosis assistance information may be trained through an eye image data set including a plurality of eye image data.

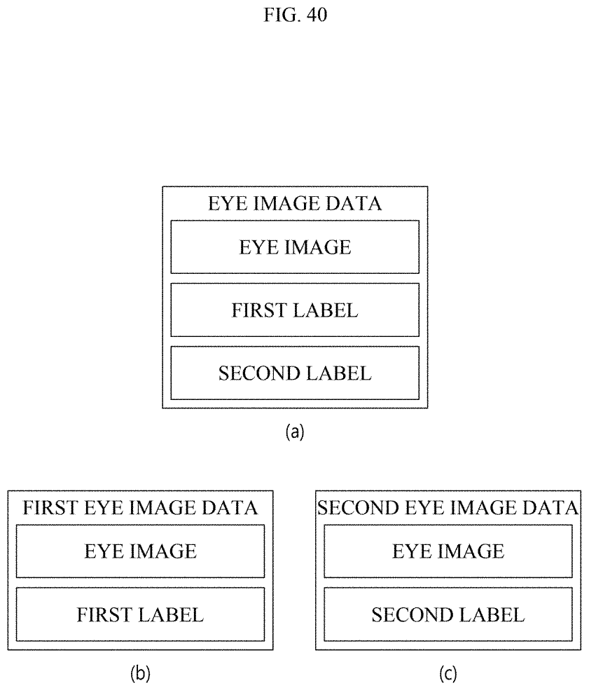

FIG. 40 is a view for describing eye image data according to an embodiment. Referring to view (a) of FIG. 40, eye image data according to an embodiment may include an eye image, first label corresponding to the eye image, and second label corresponding to the eye image. Referring to view (b) of FIG. 40, eye image data according to an embodiment may include first eye image data including an eye image and first label corresponding to the eye image, and second eye image data including an eye image and second label corresponding to the eye image.

The first label may be first label that corresponds to first diagnosis assistance information. For example, the first label may be a diagnosis information label that is used for a diagnosis of first disease, such as the presence of a disease, a degree of disease risk, numerical information related to a disease, score information related to a disease, object information associated with a disease (height, smoking status, age, gender, etc.).

The second label may be a label that corresponds to second diagnosis assistance information. For example, the second label may be a diagnosis information label that is used for a diagnosis of second disease, such as the presence of a disease, a degree of disease risk, numerical information related to a disease, score information related to a disease, object information associated with a disease (left or right eye, height, smoking status, age, gender, etc.).

The second label may be a label regarding the second disease which is different from the first disease. However, the invention described in the present specification is not limited thereto. For example, the first label and the second label may be different object information. For example, the first label may be age information of a subject and the second label may be gender information of the subject.

The eye image data may include more labels than those illustrated in FIG. 40. For example, the eye image data may also include a diagnosis information label related to a diagnosis of a disease, an ID label for identifying a subject, a gender label indicating gender of a subject.

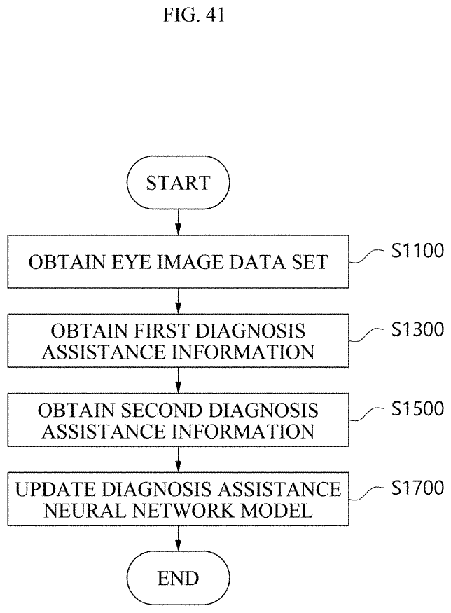

FIG. 41 is a view for describing a training method a diagnosis assistance neural network model according to an embodiment of the invention described in the present specification. Referring to FIG. 41, the training method of the diagnosis assistance neural network model according to an embodiment may include obtaining an eye image data set (S1100), obtaining first diagnosis assistance information (S1300), obtaining second diagnosis assistance information (S1500), and updating the diagnosis assistance neural network model (S1700).

The obtaining of the eye image data set (S1100) may include obtaining a data set including an eye image and at least one label, which has been described in the present specification (FIG. 40). The obtaining of the eye image data set (S1100) may include obtaining an eye image data set including eye image data including first label and second label. The obtaining of the eye image data set (S1100) may include obtaining first eye image data set including first eye image data including first label, and/or second eye image data set including second eye image data including second label.

The obtaining of the first diagnosis assistance information (S1300) may include obtaining the first diagnosis assistance information corresponding to the eye image through the diagnosis assistance neural network model. The first diagnosis assistance information may be information that corresponds to the first label and is obtained by the diagnosis assistance neural network model.

The obtaining of the second diagnosis assistance information (S1500) may include obtaining the second diagnosis assistance information corresponding to the eye image through the diagnosis assistance neural network model. The second diagnosis assistance information may be information that corresponds to the second label and is obtained by the diagnosis assistance neural network model.

The updating of the diagnosis assistance neural network model (S1700) may include updating a neural network layer associated with the first diagnosis assistance information, based on the first diagnosis assistance information and the first label. The updating of the diagnosis assistance neural network model (S1700) may include updating a neural network layer associated with the second diagnosis assistance information, based on the second diagnosis assistance information and the second label. The updating of the diagnosis assistance neural network model (S1700) may include updating the diagnosis assistance neural network model through error backpropagation or gradient descent (stochastic gradient descent, momentum, Nesterov momentum, AdaGrad, RMSprop, Adam, etc.), based on a difference between diagnosis assistance information and a label.

The method for training the diagnosis assistance neural network model may include repeatedly performing the obtaining of the first diagnosis assistance information (S1300), the obtaining of the second diagnosis assistance information (S1500), and the updating of the diagnosis assistance neural network model (S1700). The method for training the diagnosis assistance neural network model may include satisfying steps S1300 to S1700 until accuracy of the diagnosis assistance neural network model satisfies a predetermined level or higher.

The obtaining of the first diagnosis assistance information (S1300) or the obtaining of the second diagnosis assistance information (S1500) may be performed in a different order from that illustrated in FIG. 41. For example, each piece of diagnosis assistance information may be obtained with the step of updating the diagnosis assistance neural network model (S1700). The case in which the training of the diagnosis assistance neural network model is performed by updating respective portions of the neural network model after diagnosis assistance information is obtained has been described with reference to FIG. 41, but this is not an essential configuration. For example, the training method of the diagnosis assistance neural network model may include updating first individual portion (see FIG. 31) configured to obtain first diagnosis assistance information after obtaining the first diagnosis assistance information, and updating second individual portion (see FIG. 31) after obtaining second diagnosis assistance information after updating the first individual portion.

Hereinafter, some embodiments of the updating of the neural network model will be described with reference to the training method of the diagnosis assistance neural network model described in FIG. 41.

According to an embodiment of the invention described in the present specification, the diagnosis assistance neural network model which obtains a plurality of diagnosis assistance information may be collectively updated for a plurality of diagnosis assistance labels, and may be trained.

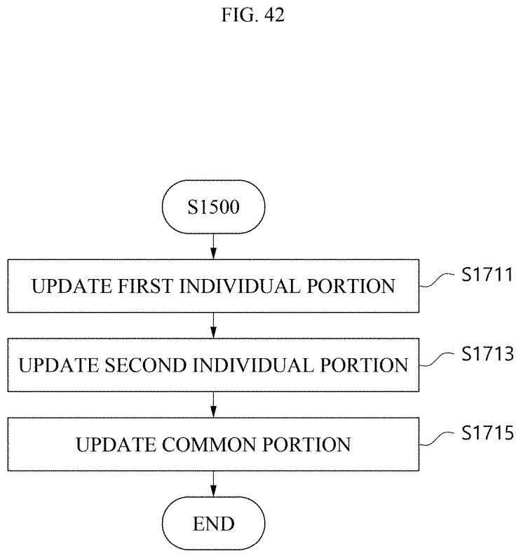

FIG. 42 is a view for describing a training method of a diagnosis assistance neural network model according to an embodiment of the invention described in the present specification. The training method of the diagnosis assistance neural network model illustrated in FIG. 42 exemplifies a method for training a diagnosis assistance neural network model illustrated in FIG. 42. Referring to FIG. 42, in the training method of the diagnosis assistance neural network model, the updating of the diagnosis assistance neural network model (S1700) may include updating first individual portion (S1711), updating second individual portion (S1713), and updating a common portion (S1715).

The updating of the first individual portion (S1711) may include comparing first diagnosis assistance information obtained by the first individual portion of the diagnosis assistance neural network model, and first label, and updating a parameter (weights or bias) of the first individual portion based on an error.

The updating of the second individual portion (S1713) may include comparing second diagnosis assistance information obtained by the second individual portion of the diagnosis assistance neural network model, and second label, and updating a parameter of the second individual portion based on an error.

The updating of the common portion (S1715) may include updating a parameter of the common portion based on the first individual portion and the second individual portion which have been updated. The updating of the parameter of the common portion based on the updated first individual portion and second individual portion may include updating the parameter of the common portion according to an average of a parameter change rate of a common portion caused by each node of a final layer (hereinafter, a layer in contact with the common portion) of the first individual portion, and a parameter change rage of a common portion caused by each node of a final layer of the second individual portion. The updating of the parameter of the common portion based on the updated first individual portion and second individual portion may include updating the parameter of the common portion by giving a predetermined weight to each of the parameter change rate of the common portion caused by each node of the final layer of the first individual portion, and the parameter change rate of the common portion caused by each node of the final layer of the second individual portion.

According to an embodiment of the invention explained in the present specification, the diagnosis assistance neural network model which obtains the plurality of diagnosis assistance information may be sequentially updated with respect to a plurality of diagnosis assistance labels and may be trained.

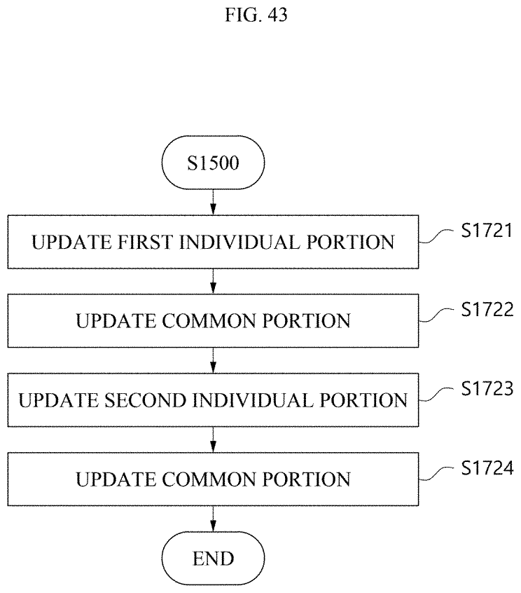

FIG. 43 is a view for describing a training method of a diagnosis assistance neural network model according to an embodiment of the invention described in the present specification. Referring to FIG. 43, the updating of the diagnosis assistance neural network model according to an embodiment may include updating first individual portion (S1721), updating a common portion (S1722), updating second individual portion (S1723), and re-updating the common portion (S1724).

The updating of the first individual portion (S1721) may be performed similarly to that described above.

The updating of the common portion (S1722) may include updating the common portion based on a difference between first diagnosis assistance information and first label. The updating of the common portion (S1722) may include updating the common portion through a parameter change rate backpropagated from each node of a final layer of the first individual portion.

Meanwhile, as described above, first label or second label may be obtained during the step of updating a neural network. For example, the updating of the second individual portion (S1723) may include updating the second individual portion based on a difference between the second label and second diagnosis assistance information which are obtained based on the updated common portion.

The re-updating of the common portion (S1724) may include updating the common portion based on the difference between the second label and the second diagnosis assistance information. The re-updating of the common portion (S1724) may include updating the common portion through a parameter change rate backpropagated from each node of a final layer of the second individual portion.

Meanwhile, in the above-described embodiment, the case in which the diagnosis assistance neural network model for predicting first diagnosis assistance information and second diagnosis assistance information is implemented in a single model form as shown in FIG. 31 has been described, but the diagnosis assistance neural network model for predicting first diagnosis assistance information and second diagnosis assistance information may be implemented as shown in FIG. 32.

According to an embodiment of the invention described in the present specification, the diagnosis assistance neural network model which obtains a plurality of diagnosis assistance information may be implemented by a plurality of diagnosis assistance neural network models having a corresponding common portion and having individual portions distinguished from each other, as illustrated in FIG. 32.

In this case, according to an embodiment, as a common portion of the diagnosis assistance neural network MODEL, a pre-trained neural network may be used. The common portion of the diagnosis assistance neural network model may be provided through a transfer learning scheme.

For example, there may be provided first diagnosis assistance neural network model that includes a common portion configured to obtain first feature set based on an eye image and first individual portion configured to obtain first diagnosis assistance information based on a feature set, and that is trained based on eye image training data including first diagnosis assistance label and an eye image. In this case, the common portion extracted from the first diagnosis assistance neural network model may be used as a common portion for a neural network model configured to obtain second diagnosis assistance information. For example, there may be provided second diagnosis assistance neural network model that includes the common portion of the first diagnosis assistance neural network model and second individual portion, and that is trained based on eye image data including an eye image and second diagnosis assistance label.

The first diagnosis assistance neural network model including the common portion and the first individual portion, and the second diagnosis assistance neural network model including the common portion and the second individual portion may be sequentially trained. For example, the first diagnosis assistance neural network model may be trained by repeatedly performing updating of the first individual portion and updating of the common portion. The second diagnosis assistance neural network model may include the common portion obtained from the trained first diagnosis assistance neural network model and the second individual portion, and may be trained by repeatedly performing updating of the second individual portion and updating of the common portion.

### 2.2.2 Second Type Model Training

According to an embodiment, there may be provided a training method of a diagnosis assistance neural network model in which an individual portion is positioned before and after a common portion as illustrated in FIG. 34. Hereinafter, the method will be described with reference to the embodiments of the training method described in FIGS. 41 to 43. Hereinafter, in the case of obtaining an eye image data set according to the embodiments described in FIG. 40, obtaining first diagnosis assistance information and second diagnosis assistance information, and updating a diagnosis assistance neural network, a updating method of the diagnosis assistance neural network model according to some embodiments will be described with reference to FIG. 34.

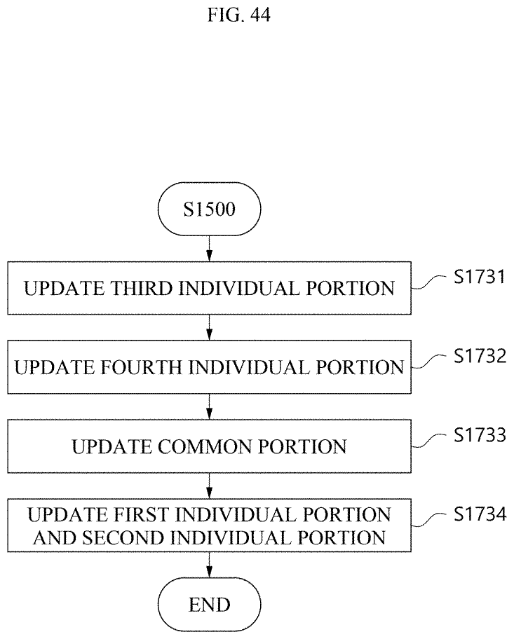

FIG. 44 is a view for describing an embodiment of a training method of a diagnosis assistance neural network model described in the present specification. Referring to FIG. 44, the updating of the diagnosis assistance neural network model may include updating third individual portion (1731), updating fourth individual portion (S1732), updating a common portion (S1733), and updating first individual portion and second individual portion (S1734).

Unless particularly mentioned otherwise, the updating of third individual portion (S1731) and the updating of the fourth individual portion (S1732) may be performed similarly to the updating of the first individual portion and the updating of the second individual portion described above in FIGS. 42 to 43.

The updating of the third individual portion (S1731) may include updating the third individual portion based on a difference between first label and first diagnosis assistance information. The updating of the fourth individual portion (S1732) may include updating the fourth individual portion based on a difference between second label and second diagnosis assistance information.

The updating of the common portion may include updating the common portion based on the difference between the first label and the first diagnosis assistance information and the difference between the second label and the second diagnosis assistance information. The updating of the common portion may be performed similarly to that described above in FIGS. 42 to 43.

The updating of the first individual portion and the second individual portion (S1734) may include updating the first individual portion and/or the second individual portion, based on the difference between the first label and the first diagnosis assistance information. The updating of the first individual portion and the second individual portion (S1734) may include updating the first individual portion and/or the second individual portion, based on the difference between the second label and the second diagnosis assistance information. The updating of the first individual portion and the second individual portion (S1734) may include updating a parameter of the first individual portion and/or the second individual portion, based on a combination of a parameter change caused by the difference between the first label and the first diagnosis assistance information and a parameter change caused by the difference between the second label and the second diagnosis assistance information.

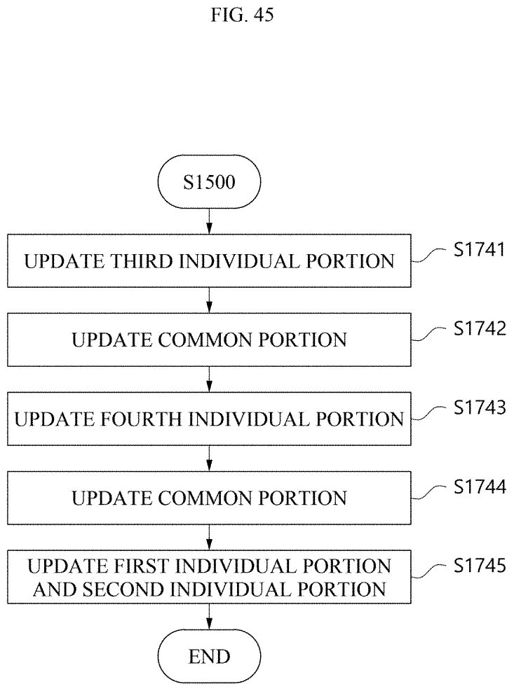

FIG. 45 is a view for describing an embodiment of a training method of a diagnosis assistance neural network model described in the present specification.

Referring to FIG. 45, the training method of the diagnosis assistance neural network model according to an embodiment may include updating third individual portion (S1741), updating a common portion (S1742), updating fourth individual portion (S1743), re-updating the common portion (S1744), and updating first individual portion and second individual portion (1745).

The updating of the third individual portion (S1741) may be performed similarly to that described above. The updating of the common portion (S1742) may be performed similarly to that described above. The updating of the common portion may include updating the common portion according to a difference between first diagnosis assistance information and first label.

The updating of the fourth individual portion (S1743) may be performed similarly to that described above. Obtaining of second label which has been described in relation with FIG. 41 may be performed within the training of the diagnosis assistance neural network model. For example, the updating of the fourth individual portion (S1743) may include updating a parameter of the fourth individual portion based on a difference between the second label and second diagnosis assistance information which are obtained through the common portion updated based on the difference between the first label and the first diagnosis assistance information.

The re-updating of the common portion (S1744) may include updating the parameter of the common portion based on the difference between the second diagnosis assistance information and the second label. The re-updating of the common portion may include re-updating the common portion based on the difference between the second label and the second diagnosis assistance information which are obtained through the common portion updated based on the difference between the first label and the first diagnosis assistance information, as described above.

The updating of the first individual portion and the second individual portion (S1745) may be implemented similarly to that described above.

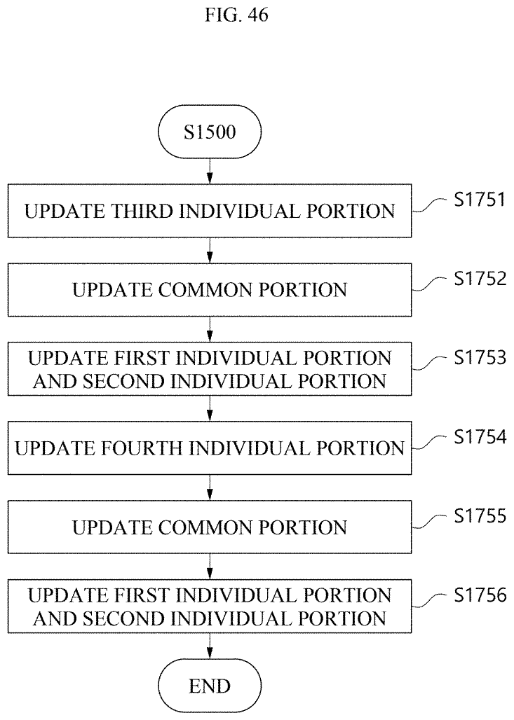

FIG. 46 is a view for describing an embodiment of a training method of a diagnosis assistance neural network model described in the present specification.

Referring to FIG. 46, the training method of the diagnosis assistance neural network model may include updating third individual portion (S1751), updating a common portion (S1752), updating first individual portion and second individual portion (S1753), updating fourth individual portion (S1754), re-updating the common portion (S1755), and updating the first individual portion and the second individual portion (S1756). The respective steps may be implemented similarly to those in the above-described embodiments.

Compared to that in FIG. 45, the updating of the first individual portion and the second individual portion may be performed after the updating of the common portion (S1752) and the re-updating of the common portion (S1755).

The updating of the first individual portion and the second individual portion (S1753) may include updating the first individual portion and the second individual portion based on a difference between first label and first diagnosis assistance information. The updating of the first individual portion and the second individual portion (S1756) may include updating the first individual portion and the second individual portion based on a difference between second label and second diagnosis assistance information.

Meanwhile, according to an embodiment, there may be provided a training method of first diagnosis assistance neural network model and second diagnosis assistance neural network model as illustrated in FIG. 35.

According to an embodiment, there may be provided a method for training first diagnosis assistance neutral network model which includes first individual portion, a common portion, and third individual portion, and second diagnosis assistance neutral network model which includes second individual portion, a common portion, and fourth individual portion.

The training method of the diagnosis assistance neutral network model according to an embodiment may include updating the third individual portion of the first diagnosis assistance neural network model (S1751), updating the common portion of the first diagnosis assistance neutral network model (S1752), updating the first individual portion of the first diagnosis assistance neural network model (S1753), updating the fourth individual portion of the second diagnosis assistance neural network model (S1754), updating the common portion of the second diagnosis assistance neural network model (S1755), and updating the second individual portion of the second diagnosis assistance neural network model (S1756). The respective steps may be performed similarly to those in the above-described embodiments.

The updating of the common portion of the second diagnosis assistance neural network model (S1755) may include transfer-training the common portion of the first diagnosis assistance neural network model. In other words, the updating of the common portion of the second diagnosis assistance neural network model (S1755) may include updating by fine-tuning the common portion of the second diagnosis assistance neural network model, which is provided by transferring the common portion of the first diagnosis assistance neural network model, using as a pre-trained model, reusing some layers, or performing domain adaption.

### 2.2.3 Third Type Model Training

According to an embodiment, there may be provided a training method of a diagnosis assistance neural network model including first common portion and second common portion as illustrated in FIGS. 37 to 39. Hereinafter, the method will be described with reference to the embodiments of the training method described above with reference to FIGS. 41 to 46.

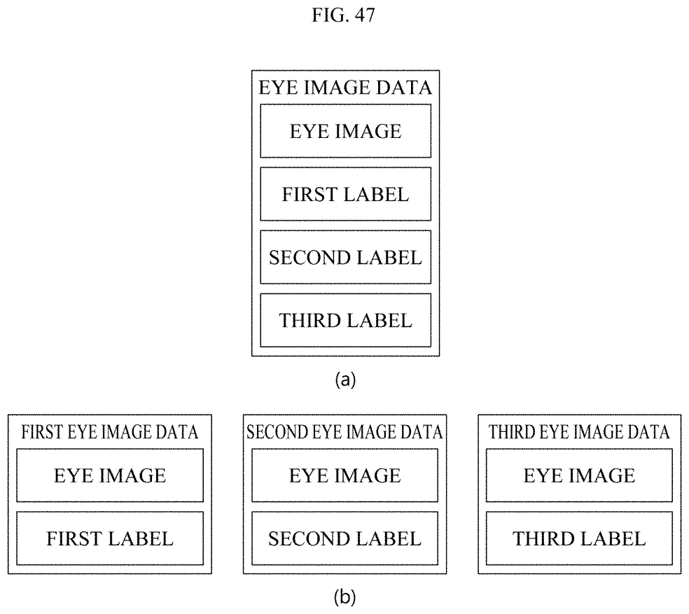

FIG. 47 is a view for describing eye image data according to some embodiments.

Referring to FIG. 47, obtaining an eye image data set may include obtaining an eye image data set which includes eye image data including first label, second label, and third label.

Alternatively, the obtaining of the eye image data set may include obtaining first eye image data set which includes first eye image data including first eye image and first label, second eye image data set which includes second eye image data including second eye image and second label, and third eye image data set which includes third eye image data including third eye image and third label.

Hereinafter, a method for training a diagnosis assistance neural network model based on an eye image data set according to the embodiment illustrated in FIG. 47 will be described.

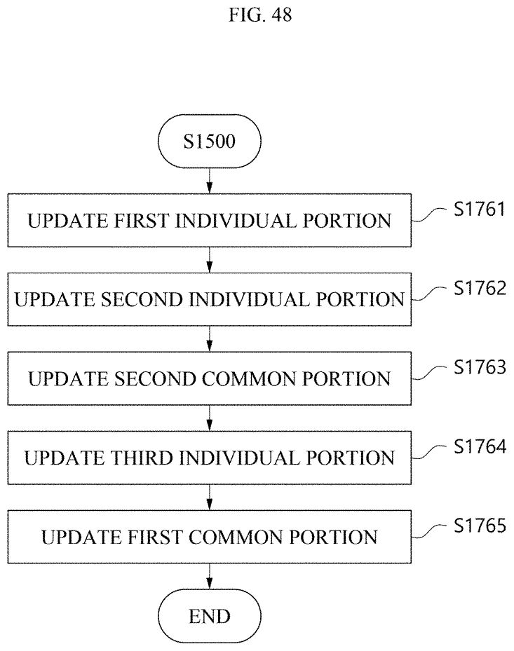

FIG. 48 is a view for describing an embodiment of a training method of a diagnosis assistance neural network model described in the present specification.

Referring to FIG. 48, the training method of the diagnosis assistance neural network model according to an embodiment may include updating first individual portion (S1761), updating second individual portion (S1762), updating second common portion (S1763), updating third individual portion (S1764), and updating first common portion (S1765). The respective steps may be implemented similarly to those in the above-described embodiments.

The updating of the first individual portion (S1761) may include updating a parameter of the first individual portion based on a difference between first label and first diagnosis assistance information. The updating of the second individual portion (S1762) may include updating a parameter of the second individual portion based on a difference between second label and second diagnosis assistance information.

The updating of the second common portion (S1763) may include updating a parameter of the second common portion based on the difference between the first label and the first diagnosis assistance information and the difference between the second label and the second diagnosis assistance information.

The updating of the third individual portion (S1764) may include updating a parameter of the third individual portion based on a difference between third label and third diagnosis assistance information.

The updating of the first common portion (S1765) may include updating the first common portion based on the difference between the first label and the first diagnosis assistance information, the difference between the second label and the second diagnosis assistance information, and the difference between the third label and the third diagnosis assistance information.

The updating of the first common portion (S1765) may include updating a parameter of the first common portion based on a change rate of the parameter of the second common portion and a change rate of the parameter of the third individual portion. The updating of the first common portion (S1765) may include updating the parameter of the first common portion by giving a greater weight to the change rate of the parameter of the second common portion than to the change rate of the parameter of the third individual portion.

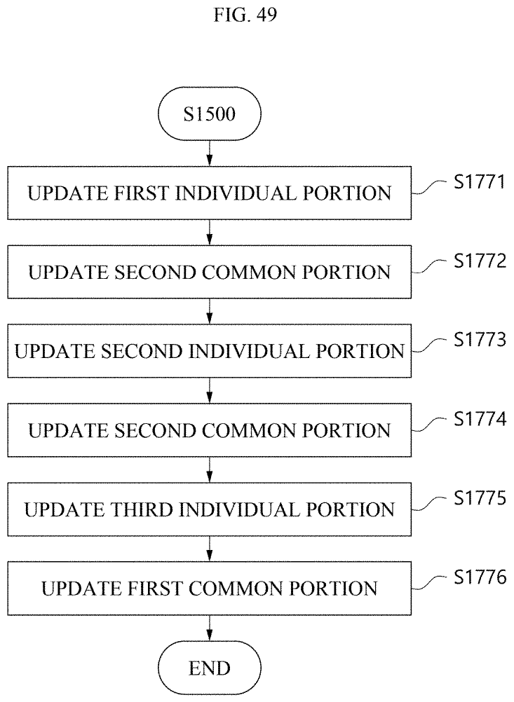

FIG. 49 is a view for describing an embodiment of a training method of a diagnosis assistance neural network model described in the present specification.

Referring to FIG. 49, the training method of the diagnosis assistance neural network model according to an embodiment may include updating first individual portion (S1771), updating second common portion (S1772), updating second individual portion (S1733), updating the second common portion (S1773), updating third individual portion (S1774), and updating first common portion (S1755). The respective steps may be implemented similarly to those in the above-described embodiments.

Compared with the method in the embodiment of FIG. 48, the updating method may update the second common portion after updating the first individual portion, and may re-update the second common portion after updating the second individual portion.

The updating of the second individual portion (S1773) may include updating a parameter of the second individual portion based on a difference between second label and second diagnosis assistance information, which are obtained by the second common portion that is updated based on a difference between first label and first diagnosis assistance information.

The updating of the second common portion (S1774) may include updating a parameter of the second common portion based on the difference between the second label and the second diagnosis assistance information, which are obtained by the second common portion that is updated based on the difference between the first label and the first diagnosis assistance information.

The updating of the first common portion may include updating the first common portion, based on the difference between the first label and the first diagnosis assistance information, the difference between the second label and the second diagnosis assistance, and a difference between third label and third diagnosis assistance information, but a change rate of a parameter of the first common portion caused by the difference between the first label and the first diagnosis assistance information and the difference between the second label and the second diagnosis assistance information may be different from a change rate of the parameter of the first common portion caused by the difference between the third label and the third diagnosis assistance information. The change rate of the parameter of the first common portion caused by the difference between the first label and the first diagnosis assistance information and the difference between the second label and the second diagnosis assistance information may be greater than the change rate of the parameter of the first common portion caused by the difference between the third label and the third diagnosis assistance information. For example, a learning rate by error backpropagation transmitted from the second common portion to the first common portion may be greater than a learning rate by error backpropagation transmitted from the third individual portion to the first common portion. However, the invention described in the present specification is not limited thereto, and according to the number of data or a features, the change rate of the parameter of the first common portion caused by the difference between the first label and the first diagnosis assistance information and the difference between the second label and the second diagnosis assistance information may be smaller than the change rate of the parameter of the first common portion caused by the difference between the third label and the third diagnosis assistance information. The learning rate by error backpropagation transmitted from the second common portion to the first common portion may be smaller than the learning rate by error backpropagation transmitted from the third individual portion to the first common portion.

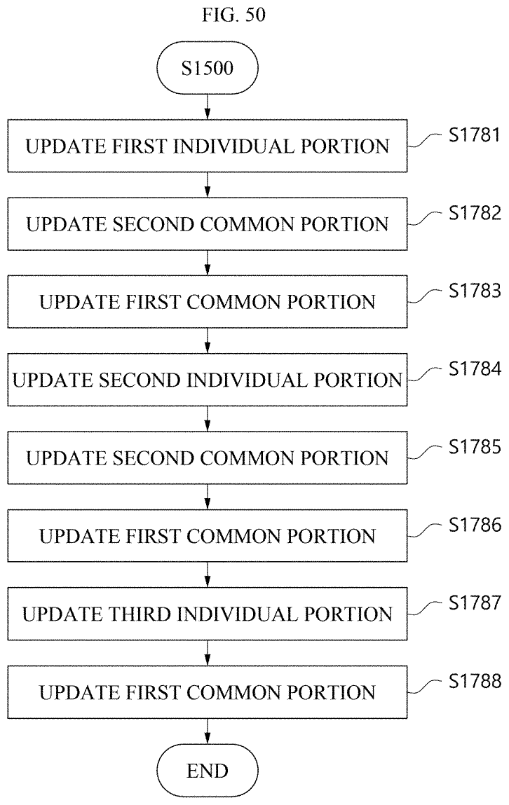

FIG. 50 is a view for describing an embodiment of a training method of a diagnosis assistance neural network model described in the present specification.

Referring to FIG. 50, the training method of the diagnosis assistance neural network model according to an embodiment may include updating first individual portion (S1781), updating second common portion (S1782), updating first common portion (S1783), updating second individual portion (S1784), updating the second common portion (S1785), updating the first common portion (S1786), updating third individual portion (S1787), and updating the first common portion (S1788). The respective steps may be implemented similarly to those in the above-described embodiments.

Compared with the method of FIG. 48 or 49, the training method according to FIG. 50 may perform first updating of the first common portion (S1783) after updating the first individual portion and the second common portion, may perform second updating of the first common portion (S1786) after updating the second individual portion and the second common portion, and may perform third updating of the first common portion (S1788) after updating the third individual portion.

In this case, a parameter change rate (or learning rate) of the first common portion at each updating operation may be different. For example, a parameter change rate of the first common portion at the first updating (S1783) or the second updating (S1786) may be lower than a parameter change rate at the third updating (S1788). Alternatively, the parameter change rate of the first common portion at the first updating (S1783) or the second updating (S1786) may be higher than the parameter change rate at the third updating (S1788).

### 2.3 Diagnosis Assistance Through a Model Obtaining a Plurality of Diagnosis Assistance Information

According to an embodiment, there may be provided a diagnosis assistance method which performs diagnosis assistance on a target disease of a subject by using a neural network model which obtains a plurality of diagnosis assistance information.

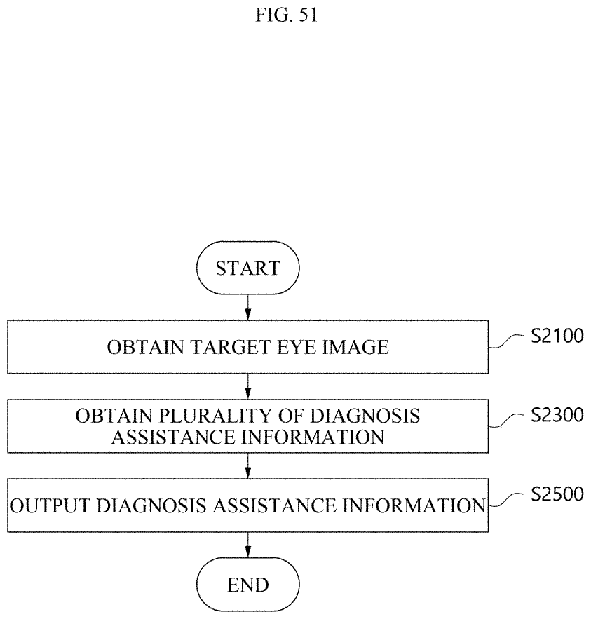

FIG. 51 is a view for describing a diagnosis assistance method according to an embodiment. Referring to FIG. 51, the diagnosis assistance method according to an embodiment may include obtaining a target eye image (S2100), obtaining a plurality of diagnosis assistance information based on the target eye image (S2300), and outputting diagnosis assistance information (S2500).

The obtaining of the target eye image (S2100) may include obtaining a target eye image that is obtained through an eye image capturing device with which an information processing device or a server communicates or which is separately provided.

The obtaining of the target eye image (S2100) may include obtaining an eye image that is obtained from eyes of an object, that is, a subject, for example, a fundus image, a retina image, an OCT image, or an iris image, etc.

According to an embodiment, the obtaining of the target eye image (S2100) may include obtaining one or more eye images. For example, the obtaining of the target eye image (S2100) may include obtaining first eye image of the subject and second eye image of the subject.

The obtaining of the target eye image (S2100) may include obtaining a plurality of same kinds of eye images. For example, the obtaining of the target eye image (S2100) may include obtaining the first eye image of the subject and the second eye image of the subject, and the first eye image may be first fundus image and the second eye image may be second fundus image. In this case, the diagnosis assistance method may include obtaining diagnosis assistance information based on the first fundus image and the second fundus image.

Alternatively, the obtaining of the target eye image (S2100) may include obtaining a plurality of different kinds of eye images. For example, the obtaining of the target eye image (S2100) may include obtaining the first eye image of the subject and the second eye image of the subject, and the first eye image may be a fundus image and the second eye image may be an OCT image. In this case, the diagnosis assistance method may include obtaining diagnosis assistance information based on the fundus image and the OCT image.

Alternatively, the obtaining of the target eye image (S2100) may include obtaining the first eye image of the subject and the second eye image of the subject, and the first eye image may be an eye image of the left eye of the subject, and the second eye image may be an eye image of the right eye of the subject. In this case, the diagnosis assistance method may include obtaining diagnosis assistance information based on the left eye image and the right eye image.

The target eye image may be an image that is obtained by performing pre-processing with respect to one or more images. The target eye image may be an image in which one or more eye-related images overlap. The target eye image may be an image in which one or more eye-related images are connected.

The obtaining of the target eye image (S2100) may further include obtaining additional data besides the target eye image.

The data additionally obtained may be an image that is not related to the eye. For example, the data additionally obtained may be an image that is obtained by capturing an organ besides eyes of the subject. For example, the obtaining of the target eye image (S2100) may include obtaining an image that is obtained by capturing lung, brain, heart, or kidney of the subject.

The data additionally obtained may be non-visual data. For example, the data additionally obtained may include non-visual information related to the body of the subject, a life habit or a target disease. For example, the obtaining of the target eye image (S2100) may include obtaining information of age, height, gender, a smoking status of the subject, a medicine taken by the subject, family medical history, blood pressure, etc. of the subject.

The obtaining of the plurality of diagnosis assistance information based on the target eye image (S2300) may include obtaining, by a processor stored in the image processing device or the server, the plurality of diagnosis assistance information based on the target eye image through a pre-stored diagnosis assistance neural network model.

The obtaining of the plurality of diagnosis assistance information based on the target eye image (S2300) may include obtaining the plurality of diagnosis assistance information through a diagnosis assistance neural network model which obtains the above-described plurality of diagnosis assistance information with a target eye image as an input.

The obtaining of the plurality of diagnosis assistance information based on the target eye image (S2300) may include obtaining first diagnosis assistance information and second diagnosis assistance information. The obtaining of the plurality of diagnosis assistance information based on the target eye image (S2300) may include obtaining the first diagnosis assistance information and the second diagnosis assistance information through a diagnosis assistance neural network model including a common portion, first individual portion and second individual portion, which has been described above. The obtaining of the plurality of diagnosis assistance information based on the target eye image (S2300) may include obtaining the first diagnosis assistance information and the second diagnosis assistance information through first diagnosis assistance neural network model including a common portion and first individual portion, and second diagnosis assistance neural network model including the common portion and second individual portion, which have been described above.

The obtaining of the plurality of diagnosis assistance information (S2300) may include obtaining first feature set which is obtained based on the target eye image through the common portion of a diagnosis assistance neural network model. The obtaining of the plurality of diagnosis assistance information (S2300) may include obtaining the first diagnosis assistance information based at least in part on the first feature set through the first individual portion. The obtaining of the plurality of diagnosis assistance information (S2300) may include obtaining the second diagnosis assistance information based at least in part on the first feature set through the second individual portion.

The first diagnosis assistance information and the second diagnosis assistance information may be different diagnosis assistance information regarding one disease. According to an embodiment, the first diagnosis assistance information may be diagnosis assistance information indicating the presence of diabetic retinopathy of a subject, and the second diagnosis assistance information may be diagnosis assistance information indicating a degree of risk of diabetic retinopathy of the subject or diagnosis assistance information indicating a probability that the subject develops diabetic retinopathy within 5 years.

The first diagnosis assistance information and the second diagnosis assistance information may be diagnosis assistance information regarding different diseases. According to an embodiment, the first diagnosis assistance information may be diagnosis assistance information related to an eye disease, and the second diagnosis assistance may be diagnosis assistance information related to a disease other than an eye disease, for example, a systematic disease. The first diagnosis assistance information may be diagnosis assistance information related to an eye disease, and the second diagnosis assistance may be diagnosis assistance information related to a cerebral cardiovascular disease.

The obtaining of the plurality of diagnosis assistance information based on the target eye image (S2300) may include obtaining first diagnosis assistance information, second diagnosis assistance information, and third diagnosis assistance information. The obtaining of the plurality of diagnosis assistance information based on the target eye image (S2300) may include obtaining the first diagnosis assistance information, the second diagnosis assistance information, and the third diagnosis assistance information, through a diagnosis assistance neural network model including first common portion, second common portion, first individual portion, second individual portion, and third individual portion, which has been described above.

The obtaining of the plurality of diagnosis assistance information based on the target eye image (S2300) may include obtaining the first diagnosis assistance information through first diagnosis assistance neural network model including first common portion, second common portion, and first individual portion, which has been described, obtaining the second diagnosis assistance information through second diagnosis assistance neural network model including the first common portion, the second common portion, and second individual portion, and obtaining the third diagnosis assistance information through third diagnosis assistance neural network model including the first common portion and third individual portion.

The obtaining of the plurality of diagnosis assistance information (S2300) may include obtaining first feature set which is obtained based on the target eye image through the first common portion. The obtaining of the plurality of diagnosis assistance information (S2300) may include obtaining second feature set based at least in part on the first feature set through the second common portion. The obtaining of the plurality of diagnosis assistance information (S2300) may include obtaining the first diagnosis assistance information based at least in part on the second feature set through the first individual portion. The obtaining of the plurality of diagnosis assistance information (S2300) may include obtaining the second diagnosis assistance information based at least in part on the second feature set through the second individual portion. The obtaining of the plurality of diagnosis assistance information (S2300) may include obtaining the third diagnosis assistance information based at least in part on the first feature set through the third individual portion.

According to an embodiment, the first to third diagnosis assistance information may be different diagnosis assistance information regarding one disease. According to an embodiment, the first to third diagnosis assistance information may be different diagnosis assistance information regarding different diseases.

According to an embodiment, the first diagnosis assistance information may be diagnosis assistance information regarding first disease belonging to first disease group, and the second diagnosis assistance information may be diagnosis assistance information regarding second disease belonging to the first disease group. The third diagnosis assistance information may be diagnosis assistance information regarding third disease belonging to second disease group which is distinguished from the first disease group. Each disease group may be classified according to a lesion. For example, each disease group may be any one of an eye disease group, a cerebral cardiovascular disease group, a circulatory disease group, a gastrointestinal disease group, a heart-lung disease group.

For example, the first diagnosis assistance information may be diagnosis assistance information related to glaucoma belonging to the eye disease group, the second diagnosis assistance information may be diagnosis assistance information related to macular degeneration belonging to the eye disease group, and the third diagnosis assistance information may be diagnosis assistance information related to a coronary artery disease belonging to the cerebral cardiovascular disease group.

According to an embodiment, the first and second diagnosis assistance information may be different diagnosis assistance information regarding first disease, and the third diagnosis assistance information may be diagnosis assistance information regarding second disease which is different from the first disease.

For example, the first diagnosis assistance information may be diagnosis assistance information indicating a degree of risk of diabetic retinopathy of a subject, the second diagnosis assistance information may be diagnosis assistance information indicating the presence of diabetic retinopathy of the subject, and the third diagnosis assistance information may be diagnosis assistance information related to a coronary artery disease.

Alternatively, the first diagnosis assistance information may be diagnosis assistance information regarding first disease, the second diagnosis assistance information may be diagnosis assistance information regarding second disease which is different from the first disease, and the third diagnosis assistance information may be diagnosis assistance information regarding third disease which is different from the first and second diseases.

For example, the first diagnosis assistance information may be diagnosis assistance information related to glaucoma, the second diagnosis assistance information may be diagnosis assistance information related to diabetic retinopathy, and the third diagnosis assistance information may be diagnosis assistance information related to cataract.

For example, diagnosis assistance neural network architectures that obtain diagnosis assistance information regarding the same disease or diagnosis assistance information regarding the same disease group may have more common layers than a diagnosis assistance neural network model architecture that obtains diagnosis assistance information regarding different diseases.

The outputting of the diagnosis assistance information (S2500) may include outputting, by a control unit of the information processing device or the server, outputting the diagnosis assistance information through an output means. The outputting of the diagnosis assistance information may be performed through the above-described user interface.

The outputting of the diagnosis assistance information (S2500) may further include outputting output information that is obtained based on the diagnosis assistance information obtained through the neural network model. The output information may be information that is obtained by processing the diagnosis assistance information and is provided to a user. For example, the output information may be an image indicating grade information or a degree of risk for indicating a degree of risk of a disease that is obtained based on the diagnosis assistance information.

The outputting of the diagnosis assistance information (S2500) may include outputting the first diagnosis assistance information and the second diagnosis assistance information. The outputting of the diagnosis assistance information (S2500) may include outputting the first diagnosis assistance information and/or the second diagnosis assistance information, and/or the third diagnosis assistance information that is obtained based on the first diagnosis assistance information and the second diagnosis assistance information. For example, the outputting of the diagnosis assistance information (S2500) may include outputting the third diagnosis assistance information that is obtained based on the first diagnosis assistance information indicating a probability of having a coronary artery disease and the second diagnosis assistance information indicating a coronary artery calcium score estimation value, and that indicates whether it is necessary to perform CT scanning for the coronary artery.

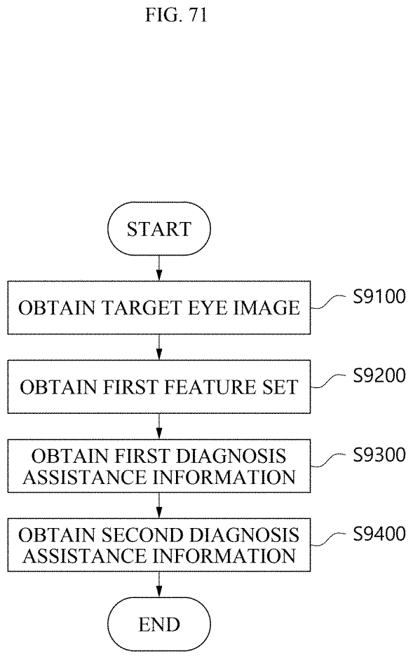

FIG. 71 is a view for describing a diagnosis assistance method according to an embodiment. Referring to FIG. 71, the diagnosis assistance method according to an embodiment may include obtaining a target eye image (S9100), obtaining first feature set (S9200), obtaining first diagnosis assistance information (S9300), and obtaining second diagnosis assistance information (S9400).

More specifically, according to an embodiment, there is provided a method for assisting a diagnosis by using a diagnosis assistance apparatus, wherein the diagnosis assistance apparatus includes: an eye image obtaining unit configured to obtain an eye image; and a processing unit configured to obtain diagnosis assistance information based on the eye image by using a neural network model, wherein the neural network model includes at least one neural network layer and is trained to obtain diagnosis assistance information based on an eye image.

The neural network model may include first diagnosis assistance neural network model configured to obtain first diagnosis assistance information based the eye image, and second diagnosis assistance neural network model configured to obtain second diagnosis assistance information based on the eye image.

The first diagnosis assistance neural network model may include first common portion and first individual portion, and the second diagnosis assistance neural network model may include the first common portion and second individual portion.

The diagnosis assistance method may include: obtaining, by the eye image obtaining unit, a target eye image that is obtained from eyes of a subject (S9100); obtaining, by the processing unit, first feature set based on the target eye image through the first common portion (S9200); obtaining, by the processing unit, first diagnosis assistance information based at least in part on the first feature set through the first individual portion (S9300); and obtaining, by the processing unit, second diagnosis assistance information based at least in part on the first feature set through the second individual portion (S9400).

The first individual portion may be trained based on first training data, and the second individual portion may be trained based on second training data which is different from the first training data at least in part.

The first individual portion may be trained based on the first training data which includes an eye image and first label. The second individual portion may be trained based on the second training data which includes an eye image and second label. The first label and the second label may include information regarding different diseases. For example, the first label may indicate the presence of a disease regarding first eye disease of a subject, and the second label may indicate the presence of a disease regarding second eye disease of the subject. Alternatively, the first label may indicate the presence of the first eye disease of the subject, and the second label may indicate the presence of first cerebral cardiovascular disease of the subject.

According to an embodiment, the first diagnosis assistance information may include at least one piece of diagnosis assistance information related to the first eye disease, and the second diagnosis assistance information may include at least one piece of diagnosis assistance information related to the second eye disease which is different from the first eye disease.

The first diagnosis assistance information may include first information and second information, and the first individual portion may include second common portion, first sub-portion, and second sub-portion.

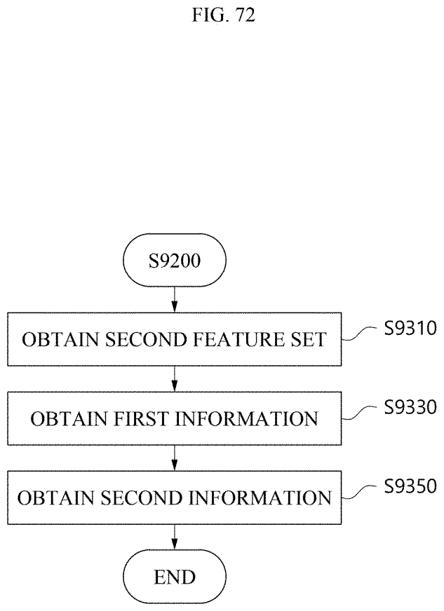

FIG. 72 is a view for describing a more specific example of the diagnosis assistance method according to an embodiment. The obtaining of the first diagnosis assistance information (S9300) may include: obtaining, by the second common portion, second feature set associated with the first information and the second information based at least in part on the first feature set (S9310); obtaining, by the first sub-portion, the first information based at least in part on the second feature set (S9330); and obtaining, by the second sub-portion, the second information based at least in part on the second feature set (S9350).

The first information and the second information may be diagnosis assistance information related to a disease related to first part of a human body, and the second diagnosis assistance information may be diagnosis assistance information related to a disease related to second part of the body. The second part may be a part that is different from the first part.

For example, the first part may be an eye and the first information may be diagnosis assistance information regarding an eye-related disease. The second part may be a heart and the second information may be diagnosis assistance information regarding a cardiovascular disease.

The first diagnosis assistance information may include diagnosis assistance information related to a disease that is included in first group disease including eye-related diseases. The second diagnosis assistance information may include diagnosis assistance information related to a disease that is included in second group disease including cerebral cardiovascular diseases. The first diagnosis assistance information or the second diagnosis assistance information may include diagnosis assistance information indicating object information regarding the subject.

The first diagnosis assistance information may include at least one piece of diagnosis assistance information related to an eye disease, and the second diagnosis assistance information may include at least one piece of diagnosis assistance information related to a cerebral cardiovascular disease.

According to an embodiment, the first feature set and/or the second feature set may include at least one feature map. For example, the first feature set may include at least one feature map and the second feature set may include at least one feature value. The first feature set and/or the second feature set may include a feature map and/or a feature value corresponding to an element included in an eye image.

Meanwhile, the processing unit may further include a pre-processing unit configured to obtain a blood vessel-emphasized eye image by performing pre-processing for emphasizing a blood vessel included in the target eye image. The obtaining of the first feature set may further include obtaining the first feature set based on the blood vessel-emphasized eye image through the first common portion.

Meanwhile, the diagnosis assistance method may be provided in the form of a computer readable recording medium having a program recorded thereon to perform the method.

### 3. Serial Connection Model

According to an embodiment, there may be provided a diagnosis assistance neural network model in which a plurality of neural network models are serially connected. Hereinafter, a serial type of diagnosis assistance neural network model will be described with reference to some embodiments.

### 3.1 Structure of Serial Diagnosis Assistance Neural Network Model

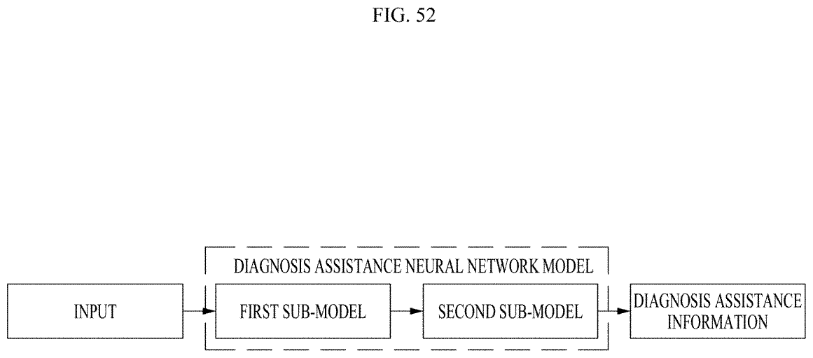

FIG. 52 is a view for describing a serial diagnosis assistance neural network model according to an embodiment. Referring to FIG. 52, the diagnosis assistance neural network model may include first sub-model and second sub-model.

The first sub-model may obtain input data including an eye image, and may obtain first output (or an intermediate output).

The first sub-model may obtain input data including an eye image and/or other images for a medical diagnosis and/or non-visual diagnosis assistance materials. The input data may include a fundus image, an OCT image, an iris image, an ophthalmology image, a lung CT image, a heart CT image, a lung X-ray image, a heart X-ray image, a kidney X-ray image, other tomographic images, an MRI image, or an X-ray image. The input data may include data indicating age, height, gender, a smoking status, family medical history of a subject.

The first output may be a value that is obtained by an output layer of the first sub-model. For example, the first sub-model may be a classifier model, and the first output may include output values at a plurality of nodes of the output layer of the first sub-model. In addition, for example, the first sub-model may be a regression model, and the first output may include a numerical value that is obtained by the first sub-model.

The first output may be a value that is provided by some layers of the first sub-model. For example, the first output may be a value that is obtained based on a value of an output layer of the first sub-model. Alternatively, the first output may be a value that is obtained based on a value of a hidden layer of the first sub-model

According to an embodiment, the first output may be a value that is obtained by an activation function on the output layer of the first sub-model. When the output layer of the first sub-model includes a plurality of nodes (or neurons), the first output may include an output value according to each of the plurality of nodes or a value that is obtained through a pre-defined function (for example, summing) based on each output value.

The activation function may be any one of a sigmoid function, a hyperbolic tangent function, a rectified linear unit (ReLu) function, PReLu, a leakey ReLU function, an identify function, an exponential linear unit (ELU) function, a Maxout function.

The first output may be a feature map or a feature value related to a target disease. The first output may be a probability map, a saliency map, a heat map, etc. related to a target disease. The second sub-model may be provided to obtain diagnosis assistance information based on the feature map or the feature value related to the target disease.

The first output may be a stochastic representation related to the target disease. For example, when the target disease is a coronary artery disease and the diagnosis assistance information is numerical information related to the target coronary artery disease, the first output may be a probability of the presence of the target coronary artery disease of a subject that is obtained based on the eye image. The second sub-model may be provided to obtain the diagnosis assistance information regarding the target disease based on the stochastic representation related to the target disease.

The second sub-model may obtain second output (or diagnosis assistance information) based on the first output. The second sub-model may be a diagnosis assistance neural network model that is trained to obtain the second output with the first output as an input. The second output may be various types of diagnosis assistance information described in the present specification.

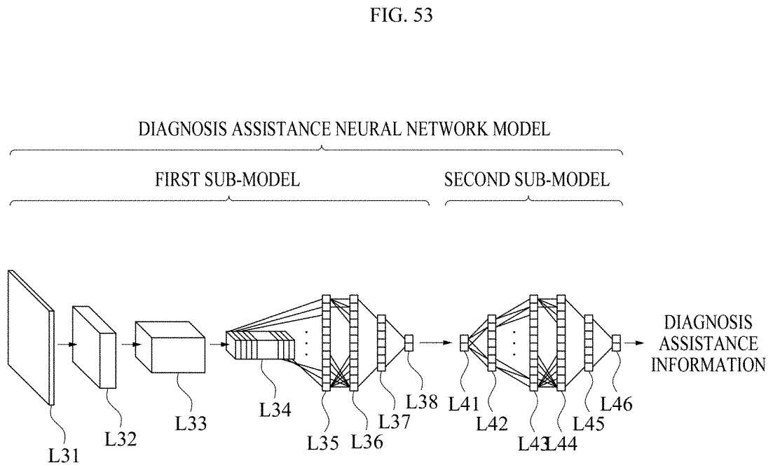

FIG. 53 is a view for describing the diagnosis assistance neural network model in more detail according to an embodiment. Referring to FIG. 53, the diagnosis assistance neural network model according to an embodiment may include the first sub-model including a plurality of layers, and the second sub-model including a plurality of layers.

Referring to FIG. 53, the first sub-model may include a plurality of convolution neural network layers. The first sub-model may include a 31 layer L31 which is an input layer according to an eye image, a 32 layer L32 which is obtained by applying first convolution filter to the 31 layer L31, a 33 layer L33 which is obtained by applying second convolution filter to the 32 layer L32, a 34 layer L34 which is obtained by applying third convolution layer to the 33 layer L33, a 35 layer L35 which is obtained by flattening the 34 layer L34, a 36 layer L36 which is fully connected with nodes included in the 35 layer L35, a 37 layer L37 which is fully connected with the 36 layer L36, and a 38 layer L38 which is an output layer fully connected with the 37 layer L37 and applying an activation function. The 38 layer L38 may provide the first output, for example, a probability output related to a target disease. The first sub-model may include more or fewer layers than those illustrated in FIG. 53.

Referring to FIG. 53, the second sub-model may include a 41 layer L41 which is an input layer including the first output provided by the first sub-model, a 42 layer L42 which is fully connected with the 42 layer, a 43 layer L43 which is fully connected with the 42 layer L42, a 44 layer L44 which is fully connected with the 43 layer, a 45 layer L45 which is fully connected with the 44 layer L44, and a 46 layer L46 which is an output layer fully connected with the 45 layer L45 and applying an activation function. The 46 layer L46 may provide the second output, for example, numerical information related to the target disease, information of a degree of risk. The second sub-model may include more or fewer layers than those illustrated in FIG. 53.

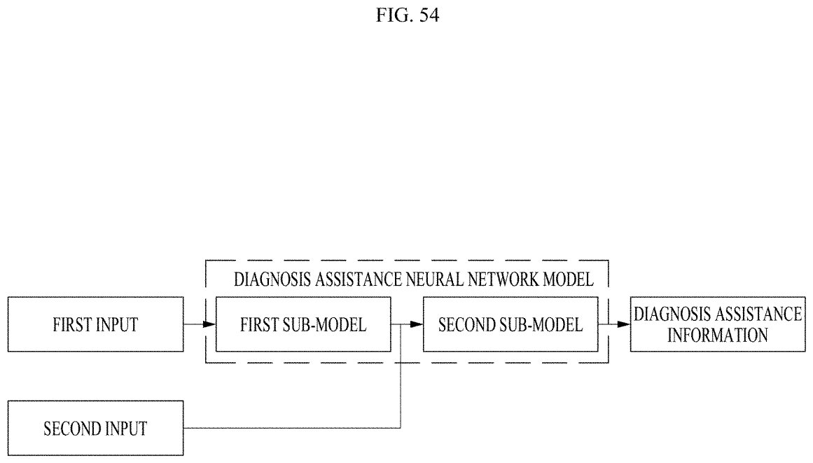

FIG. 54 is a view for describing a serial diagnosis assistance neural network model according to an embodiment. Referring to FIG. 54, the diagnosis assistance neural network model may include first sub-model and second sub-model.

The first sub-model may obtain input data including an eye image, and may obtain first output (an intermediate output). The second sub-model may obtain diagnosis assistance information based on the first output and second input.

The second input may be the same input data as the first input. For example, the first sub-model may obtain the first output based on an eye image, and the second sub-model may obtain diagnosis assistance information based on the first output and the eye image.

The second input may be input data that is obtained based on the first input. For example, the second input may be input data that is obtained by performing image processing with respect to the eye image. For example, the second input may be a monochrome eye image, a blood vessel-emphasized eye image, a blood vessel image extracted from an eye image, or an eye image without a blood vessel.

The second input may be input data that is different from the first input at least in part.

The second input may be image data that is different from the first input. The second input may be a fundus image, an OCT image, an iris image, an ophthalmology image, a lung CT image, a heart CT image, a lung X-ray image, a heart X-ray image, a kidney X-ray image, other tomographic images, an MRI image, or an X-ray image.

The second input may include non-visual information regarding a subject. For example, the first sub-model may obtain the first output related to a target disease based on the eye image, and the second sub-model may obtain diagnosis assistance information based on the first output and object information of the subject (age, gender, a smoking status, etc. of the subject). The second input may include data indicating age, height, gender, a smoking status, family medical history of the subject.

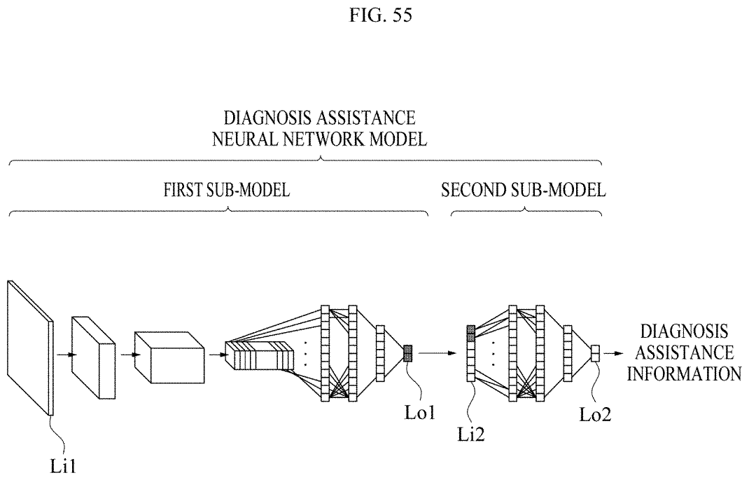

FIG. 55 is a view for describing the diagnosis assistance neural network model in more detail according to an embodiment. Referring to FIG. 55, the first sub-model may include first input layer Li1 which is generated based on the eye image, a plurality of convolution neural network layers, a plurality of fully connected layers, and first output layer Lo1. Contents of the above-described embodiments may be similarly applied to each layer.

Referring to FIG. 55, the second sub-model may include an input layer Li2 and an output layer Lo2. Referring to FIG. 55, the second sub-model may include first input layer Li2 which includes a plurality of nodes, a plurality of fully connected neural networks, and second output layer Lo2, and may obtain diagnosis assistance information based at least in part on the first output layer Lo1 of the first sub-model.

The second input layer Li2 of the second sub-model may be provided based on the first output which is obtained by the first output layer Lo1 of the first sub-model. The second input layer Li2 may include a node corresponding to the first output which is obtained by the first output layer Lo1. The node corresponding to the first output may have first output value as an input value.

The second input layer Li2 may obtain the first output which is obtained by the first output layer Lo1, and the second input as an input. The second input layer Li2 may obtain a plurality of input values, and the plurality of input values may include at least one feature value that is obtained based on a feature value or a feature map obtained from any one of the plurality of layers constituting the first sub-model.

The second output layer Lo2 may obtain diagnosis assistance information based at least in part on information obtained from the first output layer Lo1. The second output layer Lo2 may include one or more nodes and may obtain one or more output values through an activation function.

Although the case in which the first sub-model obtains the first output based on the eye image and the second sub-model obtains the diagnosis assistance information based on the first output and the non-visual information has been described with reference to FIG. 55, the invention described in the present specification is not limited thereto. For example, the diagnosis assistance neural network model according to an embodiment may be provided such that the first sub-model obtains the first output based on non-visual information, and the second sub-model obtains diagnosis assistance information based on the first output and the eye image.

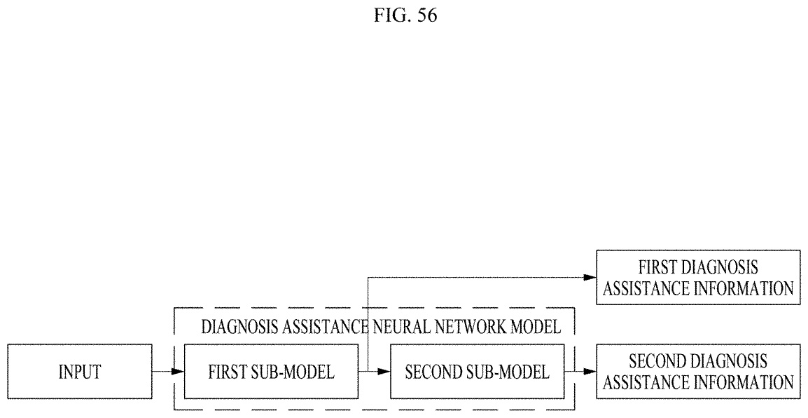

FIG. 56 is a view for describing a serial diagnosis assistance neural network model according to an embodiment. Referring to FIG. 56, the diagnosis assistance neural network model may include first sub-model and second sub-model.

Compared to the diagnosis assistance neural network model of FIG. 54, the diagnosis assistance neural network model may further obtain first diagnosis assistance information obtained by the first sub-model. The diagnosis assistance neural network model may obtain intermediate diagnosis assistance information and second diagnosis assistance information obtained based on the intermediate diagnosis assistance information.

The diagnosis assistance neural network model may obtain the first diagnosis assistance information and the second diagnosis assistance information. The first sub-model may obtain input data including an eye image, and may obtain first output (for example, the first diagnosis assistance information or intermediate output). The diagnosis assistance neural network model may obtain the first diagnosis assistance information based on the first output. The second sub-model may obtain the second diagnosis assistance information based at least in part on the first output. The diagnosis assistance neural network model may obtain the second diagnosis assistance information based at least in part on the first output by considering other information extracted from the eye image.

The first diagnosis assistance information and the second diagnosis assistance information may be diagnosis assistance information regarding the same target disease. The first diagnosis assistance information may be diagnosis assistance information that is obtained based on the eye image (clinically or through a machine-trained model), for example, a stochastic representation regarding the presence of an eye disease, a vascular anomaly, the presence of a cardiovascular disease, etc.

The second diagnosis assistance information may indicate diagnosis assistance information that is more specific than the first diagnosis assistance information. For example, the second diagnosis assistance information may include grade information indicating a degree of risk of the target disease or score information indicating a score related to the target disease, with respect to the same target disease as the first diagnosis assistance information.

The second diagnosis assistance information may correlate with the first diagnosis assistance information, but may include diagnosis assistance information that is obtained by further considering other information besides an image, for example, the progression of an eye disease, the progression of diabetic retinopathy, a coronary artery calcium score, etc.

The first diagnosis assistance information and the second diagnosis assistance information may be diagnosis assistance regarding different diseases. The first diagnosis assistance information and the second diagnosis assistance information may be diagnosis assistance information regarding different diseases belonging to the same group. For example, the first diagnosis assistance information may be diagnosis assistance information related to glaucoma belonging to the eye disease group, and the second diagnosis assistance information may be diagnosis assistance information related to macular degeneration belonging to the eye disease group. For example, the first diagnosis assistance information may be diagnosis assistance information related to drusen belonging to the eye disease group, and the second diagnosis assistance information may be diagnosis assistance information related to diabetic retinopathy belonging to the eye disease group.

The first diagnosis assistance information and the second diagnosis assistance information may be diagnosis assistance information regarding different diseases belonging to different groups. For example, the first diagnosis assistance information may be diagnosis assistance information related to macular degeneration or drusen, etc. belonging to the eye disease group, and the second diagnosis assistance information may be diagnosis assistance information related to hyperlipidemia belonging to the cerebral cardiovascular disease group.

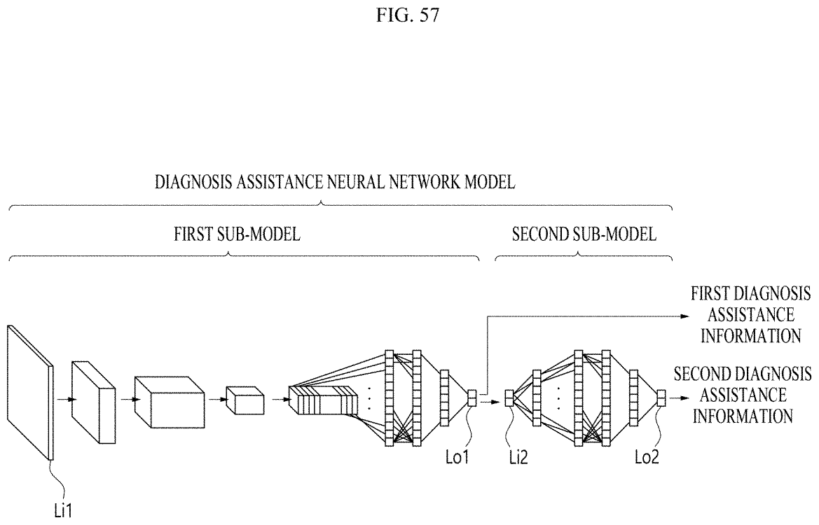

FIG. 57 is a view for describing the diagnosis assistance neural network model in more detail according to an embodiment. Unless particularly described otherwise, contents of FIGS. 53 and 55 may be similarly applied to FIG. 57.

Referring to FIG. 57, the diagnosis assistance neural network model may obtain first diagnosis assistance information through the first output layer Lo1 of the first sub-model, and may obtain second diagnosis assistance information through the second output layer Lo2 of the second sub-model. The second sub-model may obtain the second diagnosis assistance information through the first diagnosis assistance information which is obtained through the first diagnosis assistance neural network model. Alternatively, the second sub-model may obtain the second diagnosis assistance information based on the first output which is obtained through the first diagnosis assistance neural network model. The first diagnosis assistance information may be obtained by applying a predetermined function to the first output.

It is illustrated in FIG. 57 that the number of nodes of the second input layer Li2 of the second sub-model corresponds to the number of nodes of the first output layer Lo1, but this is not essential. For example, even when the first diagnosis assistance information is used as output diagnosis assistance information as shown in FIG. 57, the second input layer Li2 may further include an additional node besides nodes corresponding to the first output layer Lo1 as shown in FIG. 55.

Although the case in which the diagnosis assistance neural network model includes the first sub-model and the second sub-model has been described in the above-described embodiments, the diagnosis assistance neural network model may include more sub-models. In addition, the respective sub-models may be connected through parallel connection or serial connection described above.

### 3.2 Training of a Serial Diagnosis Assistance Neural Network Model

According to an embodiment of the invention described in the present specification, there may be provided a method for training a diagnosis assistance neural network model having serially connected sub-models as described above, through eye image training data.

Hereinafter, contents of the above-described training process may be similarly applied unless particularly described otherwise. For example, in a training process of a serial diagnosis assistance neural network model, which will be described below, the above-described data processing process such as image resizing, pre-processing, augmentation, etc. may be used. In addition, in a training process of a neural network model for predicting a plurality of diagnosis assistance information, which will be described low, the above-described training process, test, ensemble, etc. of a neural network model may be applied correspondingly.

Hereinafter, some embodiments of a training method of a neural network model for predicting a plurality of diagnosis assistance information will be described.

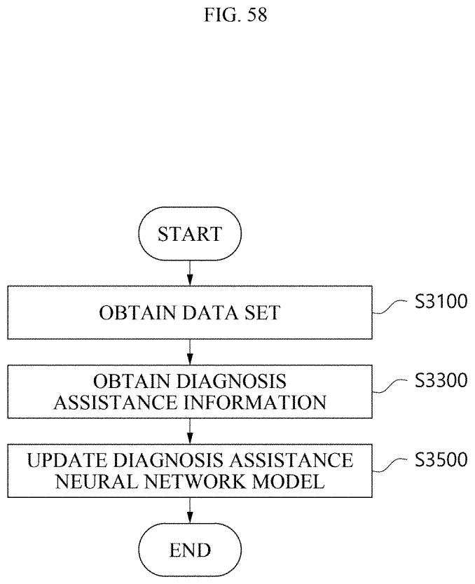

FIG. 58 is a view for describing a training method of a diagnosis assistance neural network model according to an embodiment. The training method of the diagnosis assistance neural network model illustrated in FIG. 58 may be performed by an information processing device or a control unit of the information processing device. Hereinafter, a training method of the diagnosis assistance neural network model including the first sub-model and the second sub-model, illustrated in FIG. 52, will be described.

Referring to FIG. 58, the training method of the diagnosis assistance neural network model according to an embodiment may include obtaining a data set (S3100), obtaining diagnosis assistance information (S3300), and updating the diagnosis assistance neural network model (S3500).

The obtaining of the data set (S3100) may include obtaining one or more training data sets necessary for training the first sub-model and/or the second sub-model. Eye image training data may include a plurality of eye image data. The eye image training data may be provided in the form according to embodiments described above in relation with FIG. 47.

Eye image data may include an eye image and first label corresponding to the eye image. The first label may be a label corresponding to first diagnosis assistance information obtained by the first sub-model. For example, the first label may be a grade label indicating a risk grade of a target eye disease of a subject corresponding to the eye image. In this case, the diagnosis assistance information may include risk grade information regarding the target eye disease of the subject.

The eye image data may include an eye image and second label corresponding to the eye image. The second label may be a label corresponding to the second output obtained by the second sub-model. For example, the second label may indicate the presence of the target eye disease of the subject. In this case, the first output may include a stochastic representation regarding the presence of a corresponding eye disease of the subject.

The eye image training data may further include data besides the eye image. The eye image training data may further include non-visual object information. The non-visual object information may include information regarding age, gender, a smoking status of a subject, the presence of a disease other than a target disease, family medical history regarding the target disease, the presence of high blood pressure, etc.

The obtaining of the data set (S3100) may include obtaining first training data set and second training data set. The obtaining of the eye image data set (S3100) may include obtaining first eye image data set including eye images assigned a plurality of first labels, and second eye image data set including eye images assigned a plurality of second labels. In this case, eye images included in the first eye image data set and the second eye image data set may be distinguished at least in part.

In a specific example, the obtaining of the data set (S3100) may include obtaining first eye image data set which includes a plurality of eye images assigned first label indicating the presence of first eye disease regarding first subject group, and second eye image data set which includes a plurality of eye images assigned second level indicating the presence of second eye disease regarding second subject group. The first subject group and the second subject group may be different from each other at least in part.

Alternatively, the obtaining of the data set (S3100) may include obtaining an eye image data set which is assigned a plurality of first labels and second labels.

In a specific example, the obtaining of the eye image data set (S3100) may include obtaining an eye image data set including an eye image which is assigned first label indicating the presence of first eye disease and second label indicating the presence of second eye disease regarding the first subject group.

In the above-described embodiments, the case in which the first label is related to the first eye disease and the second label is related to the second eye disease has been described, but this is not an essential configuration, and each label may be a label that is related to a disease other than the eye disease, for example, a cerebral cardiovascular disease.

The obtaining of the data set (S3100) may include obtaining first information data set which includes first information assigned the second label (information corresponding to the first diagnosis assistance information which is obtained by the first sub-model and is used for the second sub-model as an input). The first information data set may be used for training the second sub-model. In a specific example, when diagnosis assistance information obtained by the first sub-model is the first information indicating the presence (or probability) of a coronary artery disease of a subject, the first information data set may be training data including a plurality of unit data that matches the presence of the coronary artery disease of the subject and a coronary artery calcium score of the subject. The presence of the coronary artery disease of the subject which is included in the first information data set may be information that is obtained by the first sub-model or is obtained through real diagnosis.

The obtaining of the diagnosis assistance information (S3300) may include obtaining first diagnosis assistance information and/or second diagnosis assistance information. The obtaining of the diagnosis assistance information (S3300) may include obtaining the first diagnosis assistance information based on the eye image through the first sub-model. The obtaining of the diagnosis assistance information (S3300) may include obtaining the first diagnosis assistance information based on the eye image and non-visual object information. The obtaining of the diagnosis assistance information (S3300) may include obtaining the first diagnosis assistance information based on the eye image and other medical images.

The obtaining of the diagnosis assistance information (S3300) may include obtaining the second diagnosis assistance information based at least in part on the first diagnosis assistance information through the second sub-model. The obtaining of the diagnosis assistance information (S3300) may include obtaining the second diagnosis assistance information based on the first diagnosis assistance information and non-visual object information through the second sub-model. The obtaining of the diagnosis assistance information (S3300) may include obtaining the second diagnosis assistance information based on the first diagnosis assistance information and other medical images.

The updating of the diagnosis assistance neural network model (S3500) may include updating the first sub-model and/or the second sub-model. In the following description, updating/training of an entirety or a part of the neural network model may be performed by comparing information obtained through give labels and models, performing backpropagation according to an error, and optimizing weight values of an entirety or a part of the models.

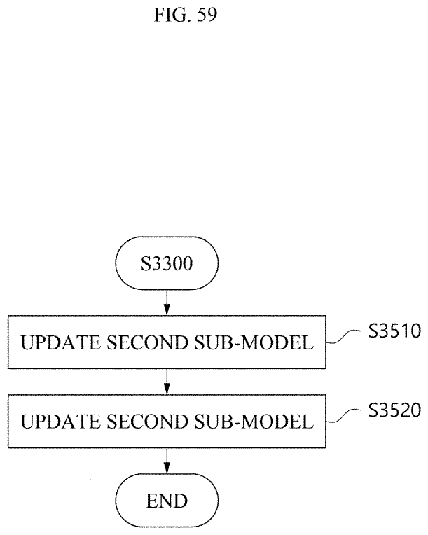

FIG. 59 is a view for describing a training method of a diagnosis assistance neural network model according to an embodiment. Referring to FIG. 59, first sub-model and second sub-model may be trained all together.

Referring to FIG. 59, the training method of the diagnosis assistance neural network model according to an embodiment may include updating the neural network model (S3500), updating the second sub-model (S3510), and updating the first sub-model (S3520).

The updating of the second sub-model (S3510) may include updating a parameter of the second sub-model based on a difference between second diagnosis assistance information and second label. The updating of the second sub-model (S3510) may include updating the second sub-model by comparing the second diagnosis assistance information that is obtained based on first diagnosis assistance information obtained based on an eye image assigned first label and second label, and the second label. The updating of the second sub-model (S3510) may include updating the second sub-model by comparing the second diagnosis assistance information that is obtained based on an eye image assigned the second label, and the second label.

The updating of the first sub-model (S3520) may include updating a parameter of the first sub-model based on a difference between the first diagnosis assistance information and the first label. The updating of the first sub-model may include updating the parameter of the first sub-model by further considering the difference between the second diagnosis assistance information and the second label. The updating of the first sub-model may include updating the first sub-model by comparing the second diagnosis assistance information that is obtained based on the first diagnosis assistance information obtained based on the eye image assigned the first label and the second label, and the second label.

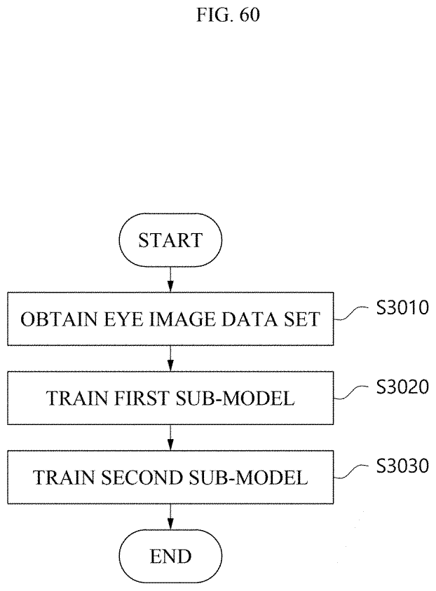

FIG. 60 is a view for describing a training method of a diagnosis assistance neural network model according to another embodiment. According to an embodiment, respective sub-models constituting the diagnosis assistance neural network model may be trained serially or individually.

Referring to FIG. 60, the training method of the diagnosis assistance neural network model according to an embodiment may include obtaining an eye image data set (S3010), training first sub-model (S3020), and training second sub-model (S3030). The above-described contents may be applied to the eye image data set.

The training of the first sub-model (S3020) may include training the first sub-model to satisfy predetermined accuracy. The training of the first sub-model may include repeatedly updating a parameter of the first sub-model based on a difference between first label and first diagnosis assistance information.

The training of the first sub-model may include training the first sub-model by using eye image training data which includes an eye image and first label corresponding to the eye image. The training of the first sub-model may include training the first sub-model by using eye image training data which includes an eye image and first label and second label corresponding to the eye image.

The training of the first sub-model may include repeating updating of the parameter of the first sub-model until the accuracy of the first sub-model is greater than or equal to a threshold value or training data is used up.

The training of the second sub-model (S3030) may include training the first sub-model to satisfy predetermined accuracy, and training the second sub-model to satisfy predetermined accuracy. The training of the second sub-model may include repeating updating of a parameter of the second sub-model until the accuracy of the second sub-model is greater than or equal to a threshold value or training data is used up.

The training of the second sub-model may be performed through eye image training data which includes an eye image and second label assigned to the eye image. The training of the second sub-model may be performed through eye image training data which includes an eye image and first label and second label assigned to the eye image. The training of the second sub-model may include updating the parameter of the second sub-model based on a difference between second diagnosis assistance information obtained through the second sub-model based on the eye image, and the second label.

The training of the second sub-model may be performed by using first information data set which includes first information and second label matching the first information. The training of the second sub-model may include updating the parameter of the second sub-model based on an error of the second diagnosis assistance information obtained based on the first information, and the second label.

The training of the second sub-model may include training the second sub-model to obtain second diagnosis assistance information based on first label which corresponds to first output obtained by the first sub-model.

The training of the second sub-model may include training the first sub-model and the second sub-model to obtain the second diagnosis assistance based on the eye image.

### 3.3 Diagnosis Assistance Through a Serial Diagnosis Assistance Neural Network Model

According to an embodiment of the invention described in the present specification, there may be provided a diagnosis assistance method through a diagnosis assistance neural network model including serially connected sub-models.

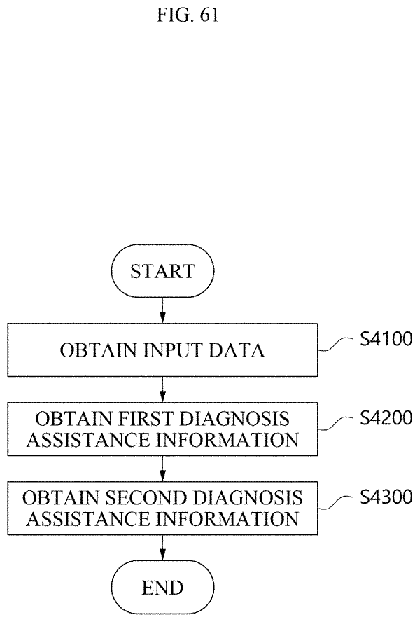

Referring to FIG. 61, the diagnosis assistance method according to an embodiment may include obtaining input data (S4100), obtaining first diagnosis assistance information (S4200), and obtaining second diagnosis assistance information (S4300).

The obtaining of the input data (S4100) may include obtaining an eye image of a subject. The obtaining of the input data (S4100) may further include obtaining a medical image regarding a body part besides eyes of the subject. The obtaining of the input data (S4100) may further include obtaining non-visual information regarding the subject. The obtaining of the input data (S4100) may further include performing pre-processing necessary for obtaining diagnosis assistance information regarding the eye image of the subject.

The obtaining of the first diagnosis assistance information (S4200) may include obtaining the first diagnosis assistance information regarding the subject based on the eye image through first sub-model. The obtaining of the first diagnosis assistance information may be obtaining diagnosis assistance information related to first disease. For example, the obtaining of the first diagnosis assistance information may include obtaining the first diagnosis assistance information indicating a probability that the subject has a target cardiovascular disease, based on the eye image.

The obtaining of the second diagnosis assistance information (S4300) may include obtaining the second diagnosis assistance information regarding the subject based on the first diagnosis assistance information through second sub-model.

The obtaining of the second diagnosis assistance information may include obtaining diagnosis assistance information that is related to the first disease and is different from the first diagnosis assistance information. For example, the obtaining of the first diagnosis assistance information may include obtaining first diagnosis assistance information indicating a probability that the subject has a target cardiovascular disease, and the obtaining of the second diagnosis assistance information may include obtaining second diagnosis assistance information indicating a score (for example, a coronary artery calcium score) that is related to whether the subject has the target cardiovascular disease based on the first diagnosis assistance information indicating the probability of the subject having the target cardiovascular disease.

Alternatively, the obtaining of the second diagnosis assistance information may include obtaining diagnosis assistance information related to second disease which is different from the first disease. For example, the obtaining of the first diagnosis assistance information may include obtaining first diagnosis assistance information indicating whether the subject has a target eye disease, and the obtaining of the second diagnosis assistance information may include obtaining second diagnosis assistance information indicating whether the subject has a cerebral cardiovascular disease.

According to an embodiment, the input data may further include data besides an eye image. For example, the input data may further include an eye image and non-visual data. The non-visual data may be non-visual object information exemplified in the present specification, for example, gender, age of a subject, etc.

The obtaining of the first diagnosis assistance information (S4200) may include obtaining the first diagnosis assistance information regarding the subject based on the eye image and the non-visual data through the first sub-model.

The obtaining of the second diagnosis assistance information (S4300) may include obtaining the second diagnosis assistance information regarding the subject based on the first diagnosis assistance information and the non-visual data through the second sub-model. For example, the obtaining of the second diagnosis assistance information may include obtaining the second diagnosis assistance information indicating a score (for example, a coronary artery calcium score) related to whether the subject has a target cardiovascular disease, based on the first diagnosis assistance information indicating a probability of the subject having the target cardiovascular disease, and age, gender, and/or a smoking status of the subject.

The diagnosis assistance through the diagnosis assistance neural network model described above will be described in detail with reference to specific embodiments.

### 4. Cerebral Cardiovascular Disease Diagnosis Assistance

Various types of diagnosis assistance neural network models described in the present specification may be used for assisting a diagnosis of a cerebral cardiovascular disease. The cerebral cardiovascular disease, which will be described below, may refer to a disease related to brain, heart or blood vessel, which includes a coronary artery disease such as heart attack or angina, a coronary heart disease, an ischaemic heart disease, a congestive heart failure, a peripheral vascular disorder, a cardiac arrest, a valvular disease of the heart, a cerebrovascular disease (for example, stroke, cerebral infarction, cerebral hemorrhage, or transient ischemic attack), and a renal vascular disease, etc. The cardiovascular disease may accompany complications. For example, the cardiovascular disease may accompany complications like a cardiac arrest, cardiac insufficiency, a stroke, an aneurysm, a peripheral arterial disease, renal insufficiency, dementia, skin ulcers. The cardiovascular disease described in the present specification may refer to such complications.

Hereinafter, unless particularly described otherwise, contents regarding the diagnosis assistance neural network model, the training method of the diagnosis assistance neural network model, or the diagnosis assistance method using the diagnosis assistance neural network model, which has been described throughout the present specification, may be similarly applied.

Hereinafter, training of a diagnosis assistance neural network model for assistance of a diagnosis of a cerebral cardiovascular disease, and assistance of a diagnosis of a cerebral cardiovascular disease through the trained diagnosis assistance neural network model will be described.

### 4.1 Cerebral Cardiovascular Disease Diagnosis Assistance Neural Network Model Structure

According to an embodiment, the cerebral cardiovascular disease diagnosis assistance neural network model may include a convolution neural network and a fully connected neural network which have been described above, and may obtain diagnosis assistance information related to a cerebral cardiovascular disease based on an eye image.

The cerebral cardiovascular disease diagnosis assistance neural network model may obtain diagnosis assistance information related to a cerebral cardiovascular disease of a subject, based on an eye image of the subject and/or a medical image of a body part other than subject's eyes and/or body information of the subject. The diagnosis assistance information related to the cerebral cardiovascular disease may include a parameter value related to the cerebral cardiovascular disease, a grade indicating a degree of risk of the cerebral cardiovascular disease, or the presence of the cerebral cardiovascular disease.

A score assisting a diagnosis of the cerebral cardiovascular disease may be a score that is measured from the subject or a score that is calculated by combining values measured from the subject and/or personal information of the subject. The score assisting the diagnosis of the cerebral cardiovascular disease may be any one of a cardiac calcification index indicating a degree of cardiac calcification, a coronary artery calcium score, an arteriosclerosis risk score, a carotid intima-media thickness (CIMT) value, a Framingham coronary artery risk score, a value of at least one factor included in a Framingham risk score, a QRISK score, a value according to an atherosclerotic cardiovascular disease (ASCVD), a score according to an European systematic coronary risk evaluation (SCORE).

The coronary artery calcium score (or cardiac calcification) may be used as a determination index regarding calcification of a coronary artery. When calcification of a coronary artery progresses as plaque accumulates in a blood vessel, a cardiovascular wall becomes narrower, causing various heart diseases such as a coronary heart disease, heart attack, angina, an ischemic heart disease, etc. Therefore, the coronary artery calcium score may be used as a base for determining a degree of risk of various heart diseases. For example, if the coronary artery calcium score is high, it may be determined that the degree of risk of a coronary artery disease is high.

In particular, the coronary artery calcium score may be directly related to a heart disease, in particular, a coronary artery disease (heart calcification), compared to factors indirectly associated with the heart disease, such as a smoking status, age, gender, and may be used as a strong biomarker regarding heart health.

A cerebral cardiovascular disease diagnosis assistance module may obtain a score value for determining the necessity for a predetermined medical prescription for treatment of a target disease, by using the cerebral cardiovascular disease diagnosis assistance neural network model. For example, the cerebral cardiovascular disease diagnosis assistance module may obtain a score value (for example, an ASCVD risk score value) for determining the necessity for prescription of statins for a subject, by using the cerebral cardiovascular disease diagnosis assistance neural network model.

Diagnosis assistance information such as a score, etc. for assisting a diagnosis of a cardiovascular disease may be used as a criterion for selecting a specific medical treatment or prescription target. For example, a coronary artery calcium score may be used for selecting a coronary artery thorough medical examination target. For example, the coronary artery calcium score may be used for selecting an antihyperlipidemic agent taking target. The coronary artery calcium score may be used as a criterion for prescription of antihyperlipidemic such as statin, etc.

In another example, the Framingham risk score value or a value used for calculating a Framingham risk score may be obtained and provided as diagnosis assistance information for determining a degree of risk of a coronary artery disease. For example, as the Framingham risk score is higher, it may be determined that the degree of risk of the coronary artery disease is higher.

In still another example, the CIMT may be obtained and provided as diagnosis assistance information for determining a degree of risk of cerebral infarction or acute myocardial infarct. For example, as the CIMT is thicker, it may be determined that the degree of risk of cerebral infarction or acute myocardial infarct is higher.

The grade assisting the diagnosis of the cerebral cardiovascular disease may be at least one grade indicating a degree of risk of the cerebral cardiovascular disease. For example, instead of a cerebral cardiovascular disease diagnosis assistance score, etc., or together with the score, the grade may be used.

The diagnosis assistance information may include a cerebral cardiovascular disease diagnosis assistance grade. The cerebral cardiovascular disease diagnosis assistance grade may include a normality grade indicating that a subject is normal for a target cerebral cardiovascular disease, and an abnormality grade indicating that a subject is abnormal for a target cerebral cardiovascular disease. In addition, the grade may include a plurality of grades indicating degrees of risk of a target cerebral cardiovascular disease of a subject.

The cerebral cardiovascular disease diagnosis assistance information may be diagnosis assistance information (for example, a value of a probability of the presence of a target disease) indicating the presence of a cerebral cardiovascular disease of a subject.

According to an embodiment, the cerebral cardiovascular disease diagnosis assistance neural network model may be provided in the form of a classifier. The cerebral cardiovascular disease diagnosis assistance neural network model may include an output layer including a plurality of output nodes, and may classify an eye image into normality or abnormality with respect to a target cerebral cardiovascular disease.

The cerebral cardiovascular disease diagnosis assistance neural network model may include an output layer including a plurality of output nodes, and may be provided to classify eye images into a plurality of groups. The cerebral cardiovascular disease diagnosis assistance neural network model may include an output layer including a plurality of nodes corresponding to a plurality of labels indicating degrees of risk of a cerebral cardiovascular disease (for example, a coronary artery disease or hyperlipidemia) of a subject. The cerebral cardiovascular disease diagnosis assistance neural network model may classify an inputted eye image according to the plurality of labels indicating the degrees of risk of the cerebral cardiovascular disease. The cerebral cardiovascular disease diagnosis assistance neural network model may classify an inputted eye image according to a plurality of ranges of a coronary artery calcium score.

According to an embodiment of the disclosure, there may be provided a cerebral cardiovascular disease diagnosis assistance neural network model which assists in determining the necessity for a medical action in relation to a cerebral cardiovascular disease based on an eye image.

According to an embodiment, the cerebral cardiovascular disease diagnosis assistance neural network model may be provided to classify a plurality of eye images into two classes which are distinguished according to the necessity for a specific medical action for a subject. For example, the cerebral cardiovascular disease diagnosis assistance neural network model may classify eye images into first class that requires a specific medical action, or second class that does not require a specific medical action.

According to an embodiment, there may be provided a cerebral cardiovascular disease diagnosis assistance neural network model which assists in determining whether to prescribe for a medical action related to a cerebral cardiovascular disease based on an eye image.

According to an embodiment, the cerebral cardiovascular disease diagnosis assistance neural network model may be trained as a binary neural network model, which classifies a plurality of eye images into two classes which are distinguished according to the necessity for a specific medical action for a subject. For example, the cerebral cardiovascular disease diagnosis assistance neural network model may be trained to classify eye images into first class that requires a specific medical action or second class that does not require a specific medical action.

For example, the cerebral cardiovascular disease diagnosis assistance neural network model may be trained to classify eye images into first class that requires a specific medical action shortly (for example, immediately), second class that requires a specific medical action within a predetermined period (for example, within 3 years), or third class that does not require a specific medical action.

The specific medical action may be a medical treatment or prescription related to angina, a coronary artery disease, a cardiac arrest, heart attack, cardiac insufficiency, arteriosclerosis, arrhythmia, cerebral hemorrhage, cerebral infarction, dyslipidemia, hyperlipidemia, high blood pressure, etc.

The specific medical action may include a pharmacological treatment or a non-pharmacological treatment that is recommended for improvement in a target disease of a subject.

The specific medical action may be administration of a specific medicine or pharmaceutical preparation, or prescription thereof. For example, the specific medical action may be prescription of one or more of statins (including various pharmaceutical preparations such as simvastatin, atorvastatin, resuvastatin, etc.) which are HMG-CoA reductase inhibitors, aspirin, bile acid sequestrant and nicotinic acid, omega-3 fatty acid, ezetimibe, fibrate.

The specific medical action may be changed according to a state of a subject and/or a target disease. For example, if the target disease is hypercholesterolemia, the specific medical action may be prescription of statins or other medicines (for example, ezetimibe, nicotinic acid, or bile acid sequestrant). If the target disease is hypertriglyceridemia, the specific medical action may be prescription of statins and nicotinic acid or fibrate. If the subject has diabetes and the target disease is hyperlipidemia, the specific medical action may be prescription of statins or statins and nicotinic acid or fibrate.

For example, the cerebral cardiovascular disease diagnosis assistance neural network model may be trained to classify a plurality of eye images into first class that does not require a subject to take statins or aspirin, or second class that requires a subject to take stain or aspirin. The first class may be treated similarly to first grade described in the present specification. The second class may be treated similarly to second grade described in the present specification.

For example, the cerebral cardiovascular disease diagnosis assistance neural network model may be trained to classify a plurality of eye images into first class in which taking of statins (or aspirin) is not recommended to a subject since a degree of risk of a predetermined disease (for example, a coronary artery disease) of the subject is low, or second class in which taking of statins (or aspirin) is recommended to a subject since the degree of risk of the predetermined disease of the subject is high.

In a specific example, the cerebral cardiovascular disease diagnosis assistance neural network model may be trained to classify a plurality of eye images into first class in which a specific score value (for example, a coronary artery calcification score value) is less than or equal to a reference value indicating the necessity for taking statins, or second class in which a specific score value is greater than or equal to the reference value indicating the necessity for taking statins.

In addition, for example, the cerebral cardiovascular disease diagnosis assistance neural network model may be trained to classify a plurality of eye images into first class that does not require a subject to take statins (aspirin), second class in which it is unclear whether a subject is required to take statins (for example, a target group requiring an additional diagnosis examination), or third class that requires a subject to take statins (for example, a target group that has low necessity for an additional diagnosis examination and is predicted as having an obvious benefit when statins or aspirin is taken). The first to third classes may be treated similarly to the first to third grades described in the present specification.

In a specific example, the cerebral cardiovascular disease diagnosis assistance neural network model may classify a plurality of eye images into first class in which a specific score value related to taking of statins (or aspirin) is less than or equal to first reference value, second class in which a specific score value related to taking of statins is greater than or equal to the first reference value and is less than or equal to second reference value, or third class in which a specific score value related to taking of statins is greater than or equal to the second reference value. For example, the cerebral cardiovascular disease diagnosis assistance neural network model may classify a plurality of eye images into first class in which a coronary artery calcification score value is less than or equal to first reference value (for example, 20), second class in which a coronary artery calcification score value is greater than or equal to the first reference value (for example, 20) and is less than or equal to second reference value (for example, 100), or third class in which a coronary artery calcification score value is greater than or equal to the second reference value (for example, 100). In addition, for example, the cerebral cardiovascular disease diagnosis assistance neural network model may classify a plurality of eye images into first class that does not require prescription of statins, second class that recommends first statin prescription, and third class that recommends second statin prescription (for example, prescription indicating a larger amount of statin than the first statin prescription or including an additional medicine).

In a specific example, the cerebral cardiovascular disease diagnosis assistance neural network model may classify a plurality of eye images into first class in which a 10-year ASCVD risk is less than or equal to first reference value (5%), second class in which a 10-year ASCVD risk is greater than or equal to the first reference value (5%) and is less than or equal to second reference value (7.5%), third class in which a 10-year ASCVD risk is greater than or equal to the second reference value (7.5%) and is less than or equal to third reference value (20%), and fourth class in which a 10-year ASCVD risk is greater than or equal to the third reference value (20%). Different prescription information related to taking of statins may correspond to the first to fourth classes. For example, the cerebral cardiovascular disease diagnosis assistance neural network model may classify a plurality of eye images into first class that does not recommend taking of statins, second class that needs to determine whether to take statins by additionally considering other factors (for example, a coronary artery calcification score), third class that requires medium level prescription of statin, and fourth class that requires high-level prescription of statins.

The specific score value in the above-described embodiments may be values of various scores, indexes or factors for evaluating a degree of risk of a cerebral cardiovascular disease described in the present specification.

According to another embodiment, there may be provided a cerebral cardiovascular disease diagnosis assistance neural network model which includes a primary neural network model (first sub-model) and secondary neural network model (second sub-model), and obtains a grade related to a target disease of a subject.

For example, the cerebral cardiovascular disease diagnosis assistance neural network model according to an embodiment may include the primary neural network model configured to obtain a probability that a subject has a target cerebral cardiovascular disease or a numerical value related to the target cerebral cardiovascular disease of the subject (for example, a coronary artery calcium score related to a coronary artery disease), based on an eye image and/or additional information of the subject. In addition, the cerebral cardiovascular disease diagnosis assistance neural network model may include the secondary neural network model configured to classify the subject according to a plurality of classes or grades related to the target cerebral cardiovascular disease with output information of the primary neural network model as an input.

For example, the cerebral cardiovascular disease diagnosis assistance neural network model according to an embodiment may include first sub-model configured to obtain a coronary artery disease corresponding probability of a subject based on an eye image of the subject, and secondary neural network model configured to determine a coronary artery disease risk grade of the subject based on the coronary artery disease corresponding probability of the subject, and may determine a risk grade related to the coronary artery disease of the subject.

According to an embodiment, the cerebral cardiovascular disease diagnosis assistance neural network model may obtain cerebral cardiovascular disease diagnosis assistance information by using a diagnosis numerical value of the subject besides the eye image as an input value. For example, according to an embodiment, a cerebral cardiovascular disease diagnosis assistance module may obtain cerebral cardiovascular disease diagnosis assistance information by using a diagnosis numerical value of the subject besides the eye image as an input value. For example, the cerebral cardiovascular disease diagnosis assistance neural network model may obtain cerebral cardiovascular disease diagnosis assistance information by using a cholesterol level, a triglyceride level, a low-density lipoprotein cholesterol level, a high-density lipoprotein cholesterol level, and/or a very-low-density lipoprotein cholesterol level, besides an eye image, as input data together with the eye image.

According to an embodiment, the cerebral cardiovascular disease diagnosis assistance neural network model may include a primary neural network model configured to obtain primary diagnosis assistance information regarding a target cerebral cardiovascular disease (for example, a coronary artery disease corresponding probability of a subject), and secondary neural network model configured to obtain secondary diagnosis assistance information (for example, a coronary artery calcium score of the subject) based at least in part on the primary diagnosis assistance information and serially connected with the primary neural network model.

According to an embodiment, the cerebral cardiovascular disease diagnosis assistance neural network model may be provided in the form of a regression model. The cerebral cardiovascular disease diagnosis assistance neural network model may be provided as a regression model configured to obtain numerical information which is used for a diagnosis of a target cerebral cardiovascular disease. For example, the cerebral cardiovascular disease diagnosis assistance neural network model may be provided to obtain a coronary artery calcium score based on an eye image.

According to another embodiment, the cerebral cardiovascular disease diagnosis assistance neural network model may be provided in the form of a diagnosis assistance neural network model to obtain a plurality of diagnosis assistance information. For example, the cerebral cardiovascular disease diagnosis assistance neural network model may obtain the presence of a coronary artery disease of a subject and a coronary artery calcium score, based at least in part on an eye image. For example, the cerebral cardiovascular disease diagnosis assistance neural network model may obtain the presence of a coronary artery disease of a subject and the presence of an eye disease based at least in part on an eye image.

According to still another embodiment, the cerebral cardiovascular disease diagnosis assistance neural network model may be a diagnosis assistance neural network model in which a plurality of models are serially connected. For example, the cerebral cardiovascular disease diagnosis assistance neural network model may include first sub-model configured to obtain first diagnosis assistance information, and second sub-model configured to obtain second diagnosis assistance information. The first diagnosis assistance information may indicate the presence of high blood pressure of a subject, and the second diagnosis assistance information may indicate the presence of hyperlipidemia of the subject.

In a specific example, the cerebral cardiovascular disease diagnosis assistance neural network model may include first sub-model configured to obtain first diagnosis assistance information indicating the presence of a coronary artery disease of a subject, and second sub-model configured to obtain second diagnosis assistance information indicating a coronary artery calcium score of the subject (or a score range to which the coronary artery calcium score of the subject corresponds) based on the presence of the coronary artery disease of the subject (or a probability of having the coronary artery disease).

In another specific example, the cerebral cardiovascular disease diagnosis assistance neural network model may include first sub-model configured to obtain first diagnosis assistance information indicating a probability that a coronary artery calcium score of a subject is greater than or equal to 0, and second sub-model configured to obtain second diagnosis assistance information indicating a coronary artery calcium score of the subject (or a score range to which the coronary artery calcium score of the subject corresponds).

### 4.2 Neural Network Model Training

According to an embodiment, there may be provided a method for training a cerebral cardiovascular disease diagnosis assistance neural network model. The method for training the cerebral cardiovascular disease diagnosis assistance neural network model may be performed by an information processing device or a control unit thereof.

The method for training the cerebral cardiovascular disease diagnosis assistance neural network model may include obtaining a training data set. The training data set may include an eye image and first label which is assigned to the eye image and is related to a cerebral cardiovascular disease. The training data set may include an eye image and first label and second label which are assigned to the eye image. The training data set may include first training data set which includes an eye image and first label assigned to the eye image, and second training data set which includes an eye image and second label assigned to the eye image.

The first label and/or the second label may indicate information related to a diagnosis of a cerebral cardiovascular disease. For example, the first label and/or the second label may indicate a parameter value related to the cerebral cardiovascular disease, a grade indicating a degree of risk of the cerebral cardiovascular disease, or the presence of the cerebral cardiovascular disease. In a specific example, the first label and/or the second label may be any one piece of cerebral cardiovascular disease diagnosis assistance information, such as the presence of hyperlipidemia of a subject, the presence of a coronary artery disease, a coronary artery calcification score, a Framingham risk score, a QRISK score, a degree of risk of a coronary artery disease (grade), etc.

The method for training the cerebral cardiovascular disease diagnosis assistance neural network model may include updating the parameter of the cardiovascular disease diagnosis assistance neural network model based on a label included in the training data.

According to an embodiment, the cerebral cardiovascular disease diagnosis assistance neural network model may be trained to obtain diagnosis assistance information based on an eye image. According to an embodiment, the cerebral cardiovascular disease diagnosis assistance neural network model may include a plurality of diagnosis assistance neural network models which are connected in parallel, and may be trained to obtain at least one piece of cerebral cardiovascular disease diagnosis assistance information based on an eye image. According to an embodiment, the method for training the cerebral cardiovascular disease diagnosis assistance neural network model may include training the cerebral cardiovascular disease diagnosis assistance neural network model by using the training data set including the eye image and the label which is assigned to the eye image and is related to the cerebral cardiovascular disease.

According to an embodiment, the cerebral cardiovascular disease diagnosis assistance neural network model may include at least one common portion and at least one individual portion, and may be trained to obtain at least one piece of cerebral cardiovascular disease diagnosis assistance information based on an eye image. The method for training the cerebral cardiovascular disease diagnosis assistance neural network model according to an embodiment may include training the cerebral cardiovascular disease diagnosis assistance neural network model by using the training data set which includes the eye image and the first label and the second label which are assigned to the eye image. The method for training the cerebral cardiovascular disease diagnosis assistance neural network model according to an embodiment may include training the cerebral cardiovascular disease diagnosis assistance neural network model by using the first training data set which includes the eye image and the first label assigned to the eye image, and the second training data set which includes the eye image and the second label assigned to the eye image.

According to an embodiment, the cerebral cardiovascular disease diagnosis assistance neural network model may include a plurality of sub-models which are serially connected, and may be trained to obtain at least one piece of cerebral cardiovascular disease diagnosis assistance information based on an eye image.

The method for training the cerebral cardiovascular disease diagnosis assistance neural network model according to an embodiment may include training to obtain first label and second label which are related to the cerebral cardiovascular disease, through the first training data set which includes the eye image and the first label assigned to the eye image, and the second training data set which includes the eye image and the second label assigned to the eye image. The method for training the cerebral cardiovascular disease diagnosis assistance neural network model according to an embodiment may be trained to obtain the first label and the second label which are related to the cerebral cardiovascular disease, through the training data set which includes the eye image and the first label and the second label assigned to the eye image.

In a specific example, the method for training the cerebral cardiovascular disease diagnosis assistance neural network model may include training first sub-model by using first training data set which includes an eye image and first label assigned to the eye image and indicating the presence of a coronary artery disease of a subject, and training second sub-model by using second training data set which includes an eye image and second label assigned to the eye image and indicating a coronary artery calcium score of the subject.

In another specific example, the method for training the cerebral cardiovascular disease diagnosis assistance neural network model may include training first sub-model by using first training data set which includes an eye image and first label assigned to the eye image and indicating a probability of a subject having a coronary artery disease, and training second sub-model by using second training data set which includes the probability of the subject having the coronary artery disease and second label matching the probability of the subject having the coronary artery disease and indicating a coronary artery calcium score of the subject.

In another specific example, the method for training the cerebral cardiovascular disease diagnosis assistance neural network model may include training first sub-model and second sub-model by using a training data set which includes an eye image, and first label that is assigned to the eye image and indicates a probability of a subject having a coronary artery disease and second label that is assigned to the eye image and indicates a coronary artery calcium score of the subject.

In another specific example, the method for training the cerebral cardiovascular disease diagnosis assistance neural network model may include training first sub-model to obtain a probability that a coronary artery calcium score of a subject is greater than or equal to 0 based on an eye image, by using a training data set which includes the eye image and the coronary artery calcium score of the subject assigned to the eye image. The method for training the cerebral cardiovascular disease diagnosis assistance neural network model may include training second sub-model to obtain second diagnosis assistance information indicating the coronary artery calcium score of the subject (or a numerical value range to which the coronary artery calcium score of the subject corresponds), based on the eye image and/or the probability that the coronary artery calcium score of the subject is greater than or equal to 0. The method for training the cerebral cardiovascular disease diagnosis assistance neural network model may include training the second sub-model by using a training data set which includes the probability that the coronary artery calcium score of the subject is greater than or equal to 0, which is obtained through the first sub-model, and a really measured coronary artery calcium score of the patent, which matches the probability.

In another specific example, the method for training the cerebral cardiovascular disease diagnosis assistance neural network model may include training the cerebral cardiovascular disease diagnosis assistance neural network model to classify an eye image into a normality class indicating that a subject does not have a target cerebral cardiovascular disease, or an abnormality class indicating that a risk of the target cerebral cardiovascular disease of the subject is a level at which taking of a predetermined medicine is required (a level at which a benefit obtained by taking of a medicine exceeds a loss).

For example, the method for training the cerebral cardiovascular disease diagnosis assistance neural network model may include training the cerebral cardiovascular disease diagnosis assistance neural network model by using an eye image training data set including a plurality of eye images which are assigned first label indicating that a subject is not required to take statins for a coronary artery disease, or second label indicating that the subject is required to take statins or aspirin for a coronary artery disease.

In another specific example, the method for training the cerebral cardiovascular disease diagnosis assistance neural network model may include training the cerebral cardiovascular disease diagnosis assistance neural network model to classify an eye image into any one of a plurality of classes which include first class indicating that a subject does not have a coronary artery disease or second class indicating that a subject has a coronary artery disease and taking of statins is recommended to the subject.

For example, the method for training the cerebral cardiovascular disease diagnosis assistance neural network model may include training the cerebral cardiovascular disease diagnosis assistance neural network model by using an eye image training data set including a plurality of eye images which are assigned first label indicating that a subject does not have a coronary artery disease or second label indicating that a subject has a coronary artery disease.

In another specific example, the method for training the cerebral cardiovascular disease diagnosis assistance neural network model may include training the cerebral cardiovascular disease diagnosis assistance neural network model by using an eye image training data set including a label related to specific prescription and an eye image.

For example, the method for training the cerebral cardiovascular disease diagnosis assistance neural network model may include training the cerebral cardiovascular disease diagnosis assistance neural network model by using an eye image training data set which includes a prescription label related to whether statins are taken, and an eye image. In a specific example, the eye image training data set may be an eye image training data set that includes a plurality of eye image data assigned first label indicating that it is not necessary to take statins, or second label indicating that it is necessary to take statins.

For example, the method for training the cerebral cardiovascular disease diagnosis assistance neural network model may include training the cerebral cardiovascular disease diagnosis assistance neural network model to determine the necessity for prescription of statins based on an eye image of a subject, by using a training data set including a plurality of eye images which are assigned first label indicating that prescription of statins is required shortly (for example, immediately), second label indicating that it is strongly predicted that the prescription of statins is required within a predetermined period (for example, within 3 years), or third level indicating that the prescription of statins is not required.

The method for training the cerebral cardiovascular disease diagnosis assistance neural network model may include training the cerebral cardiovascular disease diagnosis assistance neural network model by using the eye image training data set further including factor information related to target prescription. For example, the method for training the cerebral cardiovascular disease diagnosis assistance neural network model may include training the cerebral cardiovascular disease diagnosis assistance neural network model to obtain prescription information related to taking of statins, by using an eye image training data set including an eye image which is assigned a prescription label related to taking of statins and factor information (for example, family medical history, diabetes, function of kidney, diabetic complications, intake of aspirin, an obesity status, weight, height, a smoking status, gender, etc.) related to dyslipidemia the symptom of which is relieved by statins.

### 4.3 Cerebral Cardiovascular Disease Diagnosis Assistance

According to an embodiment, there may be provided a method for assisting a diagnosis of a cerebral cardiovascular disease through the above-described cerebral cardiovascular disease diagnosis assistance neural network model.

According to an embodiment, the method for assisting the diagnosis of the cerebral cardiovascular disease may include obtaining diagnosis assistance information for assisting the diagnosis of the cerebral cardiovascular disease based on a target eye image through the cerebral cardiovascular disease diagnosis assistance neural network model.

According to an embodiment, the method for assisting the diagnosis of the cerebral cardiovascular disease may include obtaining at least one piece of cerebral cardiovascular disease diagnosis assistance information based on an eye image, through a cerebral cardiovascular disease diagnosis assistance neural network model including a plurality of diagnosis assistance neural network models which are connected in parallel.

According to an embodiment, the method for assisting the diagnosis of the cerebral cardiovascular disease may include obtaining at least one piece of cerebral cardiovascular disease diagnosis assistance information based on an eye image, through a cerebral cardiovascular disease diagnosis assistance neural network model including at least one common portion and at least one individual portion.

According to an embodiment, the method for assisting the diagnosis of the cerebral cardiovascular disease may include obtaining at least one piece of cerebral cardiovascular disease diagnosis assistance information based on an eye image, through a cerebral cardiovascular disease diagnosis assistance neural network model including a plurality of sub-models which are serially connected.

In a specific example, the method for assisting the diagnosis of the cerebral cardiovascular disease may include obtaining first diagnosis assistance information indicating the presence of a coronary artery disease of a subject based on an eye image through first sub-model, and obtaining second diagnosis assistance information indicating a coronary artery calcium score of the subject based on an eye image through second sub-model.

In another specific example, the method for assisting the diagnosis of the cerebral cardiovascular disease may include obtaining first diagnosis assistance information indicating a probability that a subject has a coronary artery disease, based on an eye image, through the first sub-model, and obtaining second diagnosis assistance information indicating a coronary artery calcium score of the subject based at least in part on the first diagnosis assistance information through the second sub-model.

In another specific example, the method for assisting the diagnosis of the cerebral cardiovascular disease may include obtaining first diagnosis assistance information indicating a probability that a coronary artery calcium score of a subject is greater than or equal to 0, based on an eye image, through the first sub-model. The method for assisting the diagnosis of the cerebral cardiovascular disease may include obtaining, through the second sub-model, second diagnosis assistance information indicating a coronary artery calcium score of the subject (or a score range to which the coronary artery calcium score of the subject corresponds), based at least in part on the probability that the coronary artery calcium score is greater than or equal to 0, which is obtained through the first sub-model.

The method for assisting the diagnosis of the cerebral cardiovascular disease may include obtaining secondary information such as prescription information, indication information, prognostic information, prediction information, etc. for a user, based on obtained diagnosis assistance information.

The diagnosis assistance method may further include prescription information. The prescription information may include a kind of medicine to be administered to a user, an administration period, and an amount of administered medicine, etc. For example, the prescription information may include prescription information of antihyperlipidemic. The prescription information may include medicine information that is prescribed for a subject in relation with a target cerebral cardiovascular disease, such as hyperlipidemia medications, high blood pressure medications, antithrombotic medications. For example, the prescription information may include administration information regarding the necessity for administration/dosage/administration period of a pharmaceutical preparation such as statins (including various pharmaceutical preparations such as simvastatin, atorvastatin, resuvastatin, etc.) which are a HMG-CoA reductase inhibitor, bile acid sequestrant, nicotinic acid, etc.

The prescription information may be pre-stored to match diagnosis assistance information. The prescription information may be determined by using a database in which user's prescribing action according to diagnosis assistance information is stored. For example, prescription information related to administration of statins may be obtained by using a database in which risk grades regarding hyperlipidemia, other dyslipidemia and necessity for administration of stains according to grades are matched.

According to an embodiment, when diagnosis assistance information is score information for determining a degree of risk of a cardiovascular disease, prescription information may be obtained as secondary information for the score information. For example, when diagnosis assistance information is an ASCVD risk or SCORE score for determining dyslipidemia, primary prescription information indicating that, if an obtained score is less than or equal to a reference value, the necessity for a subject to take statins is low, may be obtained, and secondary prescription information indicating that, if an obtained score is less than or equal to the reference value, the necessity for a subject to taking statins is considerable, may be obtained.

According to an embodiment, when obtained diagnosis assistance information is a coronary artery calcification score (CACs), prescription information indicating that, if a coronary artery calcification score exceeds a reference value (for example, 100), taking of statins is recommended to a subject according to a pre-defined guideline (for example), may be obtained and prescription information indicating that, if a coronary artery calcification score does not reach the reference value, taking of statins is deferred, may be obtained.

The prescription information may be obtained through a neural network model that is trained by using training data including prescribing action information of a user according to diagnosis assistance information. In a specific example, there may be provided a prescription assistance neural network model which is trained to obtain prescription data inputted from a user in response to output of diagnosis assistance information, to obtain prescription information training data in which a prescription data label is assigned to diagnosis assistance information, and to output prescription information with the diagnosis assistance information as an input by using the obtained prescription information training data. The prescription information obtained by using the prescription assistance neural network model may be provided to the user together with or separately from the diagnosis assistance information. For example, a diagnostic device may obtain prescription data regarding a predetermined medicine (for example, statins) which is provided by a user in response to diagnosis assistance information (for example, grade information or score information) being obtained through a diagnosis assistance information obtaining module, and may obtain training data including an input eye image labeled with prescription data.

Diagnosis assistance information assisting a diagnosis of a cardiovascular disease, such as a score, etc., may be used as a criterion for selecting a specific medical treatment or a prescription target. For example, a coronary artery calcium score may be used for selecting a coronary artery thorough medical examination target. For example, the coronary artery calcium score may be used for selecting a target to take an antihyperlipidemic agent. The coronary artery calcium score may be used as a criterion for prescribing antihyperlipidemic such as statins, etc.

The diagnosis assistance method may further include obtaining indication information. The indication information may include information regarding a medical treatment method. For example, indication information for providing at least one candidate treatment which is predicted as being appropriate for a patient to a user may be obtained based on diagnosis assistance information. The indication information may indicate an examination additionally required, a next visiting time, a suggestion of a hospital to transfer, measures such as a recommended operation or treatment. The indication information may be pre-stored to match diagnosis assistance information. The indication information may be determined by using a database in which user's indication action is stored according to diagnosis assistance information.

For example, the indication information may include management guideline information related to a target cerebral cardiovascular disease, such as a life habit, exercise prescription, etc. which is recommended to a subject.

For example, the indication information may include additional examination information indicating a type of a thorough medical examination recommended. For example, when obtained diagnosis information indicates that the necessity for a subject to take statins is uncertain (for example, when score information is greater than or equal to first reference value and is less than or equal to second reference value, or when a target eye image is classified into second grade among first to third grades), the diagnostic device may obtain and/or output indication information for recommending CT scanning of a coronary artery (or ankle-brachial index, a blood vessel stiffness test (pulse wave velocity), 24 hours Holder monitoring, etc.).

The indication information may be obtained through a neural network model which is trained by using training data including indication action information according to diagnosis assistance information. In a specific example, there may be provided an indication assistance neural network model which is trained to obtain indication data inputted from a user in response to diagnosis assistance information being provided to the user, to obtain indication information training data assigned an indication data label according to diagnosis indication information, and to output indication information with diagnosis assistance information as an input by using an obtained indication information database. The indication information obtained by using the indication assistance neural network model may be provided to the user together with diagnosis assistance information or separately.

The diagnosis assistance method may further include obtaining predictive (or prognosis) information. The predictive information may include information regarding prognosis related to the target cerebral cardiovascular disease of the subject. For example, the predictive information may include death probability information indicating a probability of death within 5 years in relation with the target cerebral cardiovascular disease of the subject or a probability of death within 10 years.

According to an embodiment, respective pieces of secondary information obtained by diagnosis assistance information and/or the diagnosis assistance information may be outputted all together.

For example, predictive information, indication information or prescription information may be provided all together. For example, the secondary information may include predictive information when a subsequent procedure indicated by corresponding information is performed, together with specific indication information and prescription information.

For example, the secondary information may include first predictive information including a death probability of a subject when the subject does not take a medicine, and second prediction information including a death probability of the subject when a medicine is administered according to prescription information, which is determined according to obtained cerebral cardiovascular disease diagnosis assistance information.

In another example, the secondary information may include predictive information regarding a death probability or reduction of the death probability when a subject conforms to a guideline according to indication information determined according to obtained cerebral cardiovascular disease diagnosis assistance information.

The diagnosis assistance method of the cerebral cardiovascular disease may include providing a feature map, a saliency map obtained through the diagnosis assistance neural network model, for example, a class activation map, etc. to the user. The diagnosis assistance method of the cerebral cardiovascular disease may include providing a feature map displaying a correlation between a result obtained through the diagnosis assistance neural network model, and an input image to the user.

The diagnosis assistance method of the cerebral cardiovascular disease may include providing the diagnosis assistance information regarding the cerebral cardiovascular disease and/or secondary information obtained based on the diagnosis assistance information to the user through a graphic interface described in the present specification.

According to an embodiment, the graphic interface may include a score display unit configured to display a heart image filled by 0% if a coronary artery calcification score of a subject is 0, to display a heart image filled by 20% if a coronary artery calcification score of a subject is 1-10, to display a heart image filled by 50% if a coronary artery calcification score of a subject is 10-100, to display a heart image filled by 70% if a coronary artery calcification score of a subject is 100-400, to display a heart image filled by 90% if a coronary artery calcification score of a subject is 400 or more.

The graphic interface according to an embodiment may display prescription information associated with a degree of risk of a target cerebral cardiovascular disease of a subject. For example, the graphic interface may display prescription information associated with a score regarding the target cerebral cardiovascular disease of the subject. The graphic interface may display prescription information of statins associated with a coronary artery calcification score. The graphic interface may display the prescription information of statins associated with the coronary artery calcification score according to a pre-stored matching table. The graphic interface may display prescription information of statins which is calculated according to a coronary artery calcification score and other information of a subject (for example, an HDL cholesterol level, an LDL cholesterol level, a triglyceride level, age, gender, a smoking status, etc.), based on the pre-stored matching table.

### 5. Parameter Obtaining Neural Network Model

According to an embodiment, there may be provided a diagnosis assistance neural network model which obtains various parameters based on an eye image. The diagnosis assistance neural network model for obtaining a plurality of parameters may include various types of neural network structures described above.

The parameter obtained through the diagnosis assistance neural network model may be a parameter indicating body information of a subject, such as gender, age, height, weight, BMI, body mass, body fat percentage, body muscle mass, etc. of the subject. Alternatively, the parameter may be any one of diagnosis numerical value parameters, such as a hematocrit level, a red blood cell count, a white blood cell count, a hemoglobin level, a platelet count, a total iron binding capacity (TIBC), an iron level, a ferritin (iron storage protein) level, a total protein level, an albumin level, an aspartate transferase (AST) level, an amino transferase level, γ-GTP, γ-GT, an alkaline phosphatase (ALP) level, a globulin level, a hepatitis antigen level, a hepatitis antibody level, a glycated hemoglobin level (HbA1C), a blood urea nitrogen (BUN) level, a creatinine level, a uric acid level, a total cholesterol level, an HDL cholesterol level, an LDL cholesterol level, a triglyceride (TG) level, a bicarbonate level, a systolic blood pressure (SBP), a diastolic blood pressure (DRP), etc.

Hereinafter, the parameter obtained by the diagnosis assistance neural network model may be any one of the above-described examples unless particularly described otherwise.

Hereinafter, contents of the diagnosis assistance neural network model, the training method of the diagnosis assistance neural network model, or the diagnosis assistance method using the diagnosis assistance neural network model, which has been described throughout the present specification, may be similarly applied unless particularly mentioned otherwise.

### 5.1 Plural Parameter Obtaining Diagnosis Assistance Neural Network Model

According to an embodiment, there may be provided a plurality of diagnosis assistance neural network models which are connected in parallel and obtain a plurality of parameters.

For example, a diagnosis assistance neural network model according to an embodiment may include first sub-neural network model configured to obtain first parameter, and second sub-neural network model provided in parallel with first diagnosis assistance neural network model and configured to obtain second parameter.

According to another embodiment, there may be provided a diagnosis assistance neural network model which has at least one common portion and an individual portion.

For example, a diagnosis assistance neural network model may include a common portion, first individual portion, and second individual portion. For example, a diagnosis assistance neural network model may include first sub-neural network model including a common portion and first individual portion, and second sub-neural network model including a common portion and second individual portion. The common portion may obtain first feature set, and the first individual portion may obtain first parameter based on the first feature set. The second individual portion may obtain second parameter based on the first feature set.

In a specific example, the common portion may obtain first feature set based on an eye image, the first individual portion may obtain first diagnosis assistance information indicating a hematocrit level of a subject based on the first feature set, and the second individual portion may obtain second diagnosis assistance information indicating a red blood cell count of the subject based on the first feature set. In a specific example, the common portion may obtain first feature set based on an eye image, the first individual portion may obtain first diagnosis assistance information indicating a hematocrit level of a subject based on the first feature set, and the second individual portion may obtain second diagnosis assistance information indicating the presence of a target cerebral cardiovascular disease of the subject, based on the first feature set and body information (for example, gender, age, etc.) of the subject.

In addition, for example, a diagnosis assistance neural network model may include first common portion, second common portion associated with the first common portion, first individual portion associated with the second common portion, second individual portion associated with the second common portion, and third individual portion associated with the first common portion. The diagnosis assistance neural network model may include first sub-neural network model which includes the first common portion, the second common portion, and the first individual portion and obtains first parameter, second sub-neural network model which includes the first common portion, the second common portion, and the second individual portion and obtains second parameter, and third neural network model which includes the first common portion and the third individual portion and obtains third parameter.

In a specific example, the first common portion may obtain first feature set, the second common portion may obtain second feature set based on the first feature set, the first individual portion may obtain first diagnosis assistance information indicating the first parameter based at least in part on the second feature set, the second individual portion may obtain second diagnosis assistance information indicating the second parameter based at least in part on the second feature set, and the third individual portion may obtain third diagnosis assistance information indicating the third parameter based at least in part on the first feature set. The first parameter and the second parameter may be parameters that correlate with each other. For example, the first parameter may be a hematocrit level of the subject and the second parameter may be a hemoglobin level of the subject. The third parameter may be a parameter that has a low correlation with the first or second parameter. For example, the third parameter may be gender or age of the subject.

The first parameter and the second parameter may be parameters that belong to the same parameter group. The third parameter may be a parameter that belongs to a parameter group different from the first parameter and the second parameter. For example, the first parameter and the second parameter may be parameters that are related to blood of the subject. The third parameter may be a parameter that is related to body information of the subject, for example, gender, height, age, etc. of the subject.

According to another embodiment, there may be provided a neural network model which includes at least one serially connected sub-model and obtains at least one parameter.

For example, a diagnosis assistance neural network model may include first sub-model and second sub-model. The first sub-model may obtain first diagnosis assistance information indicating first parameter based on an eye image. The second sub-model may obtain second diagnosis assistance information indicating second parameter based on the first diagnosis assistance information. The second sub-model may obtain the second diagnosis assistance information indicating the second parameter, based on the first diagnosis assistance information and/or a medical image obtained by capturing a part of subject's body and/or body information of the subject (gender, age, family medical history regarding a target disease, etc.). In a specific example, the first sub-model may obtain the first diagnosis assistance information indicating subject's age based on the eye image, and the second sub-model may obtain the second diagnosis assistance information indicating a hematocrit level of the subject (or disease-related diagnosis assistance information such as the presence of a coronary artery disease), based on the age of the subject and the eye image of the subject.

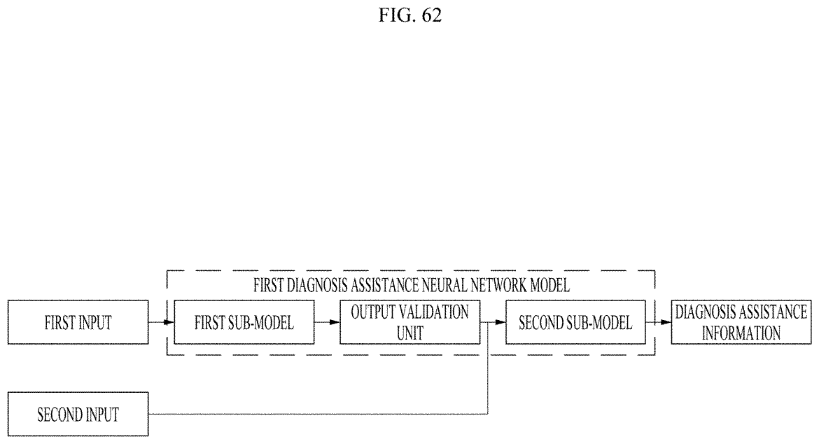

FIG. 62 is a view for describing a diagnosis assistance neural network model according to an embodiment. Referring to FIG. 62, there may be provided a diagnosis assistance neural network model which obtains diagnosis assistance information related to a plurality of parameters and/or a target disease.

Referring to FIG. 62, the diagnosis assistance neural network model may include first sub-model, an output validation unit, and second sub-model. The first sub-model and/or the second sub-model may be provided as a plurality of various types of information obtaining neural network models described in the present specification.

The first sub-model may obtain first input and may obtain first output.

The first input may include an eye image. The first input may include a pre-processed eye image. The first input may include a medical image that is obtained by capturing a part of a body of a subject, besides the eye image. The first input may include non-visual information, for example, body information of the subject.

The first output may include at least one piece of diagnosis assistance information indicating a parameter regarding the subject. For example, the first output may include first diagnosis assistance information indicating a hematocrit level of the subject, second diagnosis assistance information indicating a hemoglobin level of the subject, third diagnosis assistance information indicating the age of the subject, fourth diagnosis assistance information indicating the gender of the subject, and fifth diagnosis assistance information indicating a BMI of the subject.

The output validation unit (or output evaluation unit) may be provided as a configuration for evaluating accuracy of information obtained through a machine learning model. The output validation unit may validate the diagnosis assistance information, which is obtained through the first sub-model, by comparing with real information on the subject. For example, the output validation unit may validate the first diagnosis assistance information indicating the hematocrit level of the subject, the second diagnosis assistance information indicating the hemoglobin level of the subject, the third diagnosis assistance information indicating the age of the subject, the fourth diagnosis assistance information indicating the gender of the subject, and the fifth diagnosis assistance information indicating the BMI of the subject, which are obtained through the first sub-model, by comparing with a real hematocrit level, a real hemoglobin level, real age, real gender, and real BMI of the subject, respectively.

The output validation unit may select a part of the first output based on a result of validating. The output validation unit may transmit information of a part of the first output that is selected as the result of validating to the second sub-model. The output validation unit may select information that has accuracy of a predetermined level or higher from the diagnosis assistance information obtained through the first sub-model, and may transmit the selected information to the second sub-model. The output validation unit may select information that has accuracy of the predetermined level or higher, based on a result of comparing the diagnosis assistance information obtained through the first sub-model with real information, and may transmit the selected information to the second sub-model. Alternatively, the output validation unit may select information that has accuracy of the predetermined value or lower from the diagnosis assistance information obtained through the first sub-model, and may transmit the selected information to the second sub-model.

For example, the output validation unit may select the first diagnosis assistance information and the second diagnosis assistance information indicating accuracy of the predetermined level or higher (or lower), from the first diagnosis assistance information indicating the hematocrit level of the subject, the second diagnosis assistance information indicating the hemoglobin level of the subject, the third diagnosis assistance information indicating the age of the subject, the fourth diagnosis assistance information indicating the gender of the subject, and the fifth diagnosis assistance information indicating the BMI of the subject, which are obtained through the first sub-model.

The second sub-model may obtain at least part of the first output and second input, and may obtain second output.

The second sub-model may obtain diagnosis assistance information selected by the output validation unit from the first output. For example, the second sub-model may obtain the first diagnosis assistance information and the second diagnosis assistance information selected by the output validation unit, from the first diagnosis assistance information indicating the hematocrit level of the subject, the second diagnosis assistance information indicating the hemoglobin level of the subject, the third diagnosis assistance information indicating the age of the subject, the fourth diagnosis assistance information indicating the gender of the subject, and the fifth diagnosis assistance information indicating the BMI of the subject, which are obtained through the first sub-model.

The second input may include an eye image. The second input may include a pre-processed eye image. The second input may include a medical image that is obtained by capturing a part of the body of the subject, besides the eye image. The second input may include non-visual information, for example, body information of the subject. The second input may be omitted.

The second output may include at least one piece of diagnosis assistance information indicating a parameter regarding the subject. For example, the second output may include diagnosis assistance information indicating the presence of a target cerebral cardiovascular disease of the subject, which is obtained based on the first diagnosis assistance information indicating the hematocrit level and the second diagnosis assistance information indicating the hemoglobin level of the subject, or diagnosis assistance information indicating other parameters of the subject.

According to the diagnosis assistance neural network model including the output validation unit illustrated in FIG. 62, two types of machine learning may be utilized.

First, the second output may be obtained through the second sub-model based on diagnosis assistance information that has accuracy of the predetermined level or higher from the first output obtained through the first sub-model. In this case, high reliability may be given to the case in which information obtained by a machine learning model based on an eye image is identical to real information, so that training accuracy of the second sub-model may be further enhanced.

Alternatively, the second output may be obtained through the second sub-model based on diagnosis assistance information that has accuracy of the predetermined level or lower from the first output obtained through the first sub-model. In this case, diagnosis assistance may be performed based on a parameter indicating a value different from a real value among the parameters obtained based on the eye image, so that reliability of obtaining of diagnosis assistance information indicating abnormality of the subject may increase.

Considering this point, the output validation unit of the diagnosis assistance neural network model according to an embodiment may operate differently at a training step and a diagnosis assistance step. For example, the output validation unit may select diagnosis assistance information that has accuracy of the predetermined level or higher from the first output and may transmit the selected diagnosis assistance information to the second sub-model at the training step of the neural network model, and may select diagnosis assistance information that has accuracy of the predetermined level or lower from the first output and may transmit the selected diagnosis assistance information to the second sub-model at the diagnosis assistance step using the neural network model, or vice versa.

In the above-described embodiments, the diagnosis assistance neural network model which obtains one or more parameters has been described, but the diagnosis assistance neural network model may also obtain diagnosis assistance information related to a target disease. For example, the diagnosis assistance neural network model may obtain first parameter and second parameter through the first common portion and the second common portion, and may obtain third diagnosis assistance information indicating a degree of risk of a cerebral cardiovascular disease of the subject through the first common portion.

### 5.2 Parameter Obtaining Diagnosis Assistance Neural Network Model Training

According to an embodiment, there may be provided a method for training a diagnosis assistance neural network model to obtain one or more parameters.

The training of the diagnosis assistance neural network model to obtain parameters may include obtaining a parameter training data set including a parameter level, and training the neural network model by using the parameter training data set.

The parameter training data set may include an eye image and a parameter level assigned to the eye image. The parameter training data set may include an eye image and first parameter level and second parameter level which are assigned to the eye image. The parameter training data set may include first parameter training data set which includes an eye image and first parameter label assigned to the eye image, and second parameter training data set which includes an eye image and second parameter label assigned to the eye image. The parameter training data set may include an eye image and first label assigned to the eye image (the first label indicating a parameter related to a diagnosis of a target disease) and second label related to a target disease.

The training method of the diagnosis assistance neural network model according to an embodiment may include training first sub-neural network model which is configured to obtain first parameter, and second sub-neural network model which is provided in parallel with first diagnosis assistance neural network model and is configured to obtain second parameter, by using first training data set which includes an eye image and first label (corresponding to the first parameter) assigned to the eye image, and second training data set which includes an eye image and second label (corresponding to second parameter) assigned to the eye image.

The training method of the diagnosis assistance neural network model according to another embodiment may include training a diagnosis assistance neural network model which includes a common portion, first individual portion, and second individual portion, by using first training data set which includes an eye image and first label (corresponding to first parameter) assigned to the eye image, and second training data set which includes an eye image and second label (corresponding to second parameter) assigned to the eye image. Alternatively, the training method of the diagnosis assistance neural network model may include training a diagnosis assistance neural network model which includes a common portion, first individual portion, and second individual portion, by using a parameter training data set which includes an eye image and first label (corresponding to first parameter) and second label (corresponding to second parameter) assigned to the eye image.

In a specific example, the training method of the diagnosis assistance neural network model may include training a diagnosis assistance neural network model which includes a common portion configured to obtain first feature set based on an eye image, first individual portion configured to obtain first diagnosis assistance information indicating a hematocrit level of a subject based on the first feature set, and second individual portion configured to obtain second diagnosis assistance information indicating a red blood cell count of the subject based on the first feature set, by using first training data set which includes an eye image and first label (corresponding to first parameter) indicating a hematocrit level of the subject, and second training data set which includes an eye image and second label (corresponding to second parameter) indicating a red blood cell count of the subject.

In addition, for example, the training method of the diagnosis assistance neural network model may include training a diagnosis assistance neural network model which includes first common portion, second common portion, first individual portion, second individual portion, and third common portion, by using first training data set which includes an eye image and first label (corresponding to first parameter) assigned to the eye image, second training data set which includes an eye image and second label (corresponding to second parameter) assigned to the eye image, and third training data set which includes an eye image and third label (corresponding to third parameter) assigned to the eye image. Alternatively, the training method of the diagnosis assistance neural network model may include training a diagnosis assistance neural network model including first common portion, second common portion, first individual portion, second individual portion, and third individual portion, by using a parameter training data set which includes an eye image and first label (corresponding to first parameter), second label (corresponding to second parameter), and third label (corresponding to third parameter) assigned to the eye image.

The first parameter and the second parameter may be parameters that corelate with each other. For example, the first parameter may be a hematocrit level of the subject and the second parameter may be a hemoglobin level of the subject. The third parameter may be a parameter that has a low correlation with the first or second parameter. For example, the third parameter may be gender or age of the subject. The first parameter and the second parameter may be parameters belonging to the same parameter group. The third parameter may be a parameter that belongs to a parameter group different from the first parameter and the second parameter. For example, the first parameter and the second parameter may be parameters related to blood of the subject. The third parameter may be a parameter related to body information of the subject, for example, gender, height, age of the subject.

According to another embodiment, there may be provided a method for training a neural network model which includes at least one serially connected sub-model and obtains at least one parameter.

For example, the method for training the diagnosis assistance neural network model according to an embodiment may include training first sub-model configured to obtain first diagnosis assistance information indicating first parameter based on an eye image, by using first parameter training data set which includes an eye image and first label corresponding to the first parameter.

The training method of the diagnosis assistance neural network model may include training second sub-model configured to obtain second diagnosis assistance information indicating second parameter based on the second parameter, by using second parameter training data set which includes second label corresponding to the first parameter and the second parameter.

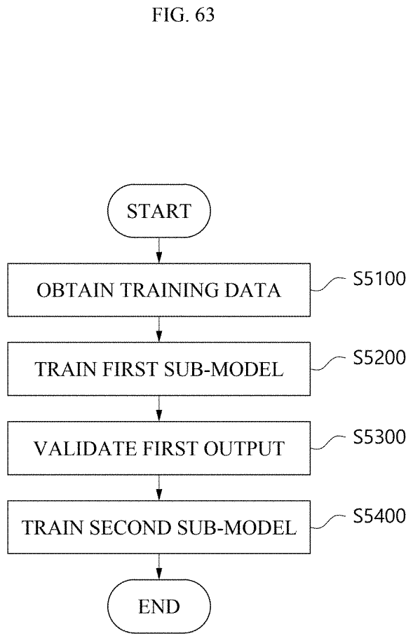

FIG. 63 is a view for describing a training method of a diagnosis assistance neural network model according to an embodiment. Referring to FIG. 63, the training method of the diagnosis assistance neural network model according to an embodiment may include obtaining training data (S5100), training first sub-model (S5200), validating first output (S5300), and training second sub-model (S5400).

The obtaining of the training data (S5100) may include obtaining a parameter training data set which includes an eye image and a plurality of parameter labels corresponding to the eye image. According to an embodiment, the parameter training data set may include an eye image and first to fifth labels corresponding to the eye image. The first label may indicate a hematocrit level of a subject. The second label may indicate a hemoglobin level of the subject. The third label may indicate age of the subject. The fourth label may indicate gender of the subject. The fifth label may indicate a BMI of the subject. Sixth label may indicate a degree of risk of a target cerebral cardiovascular disease of the subject. Seventh label may indicate a numerical value related to the target cerebral cardiovascular disease of the subject, for example, a coronary artery calcium score. The first to seventh labels may indicate information other than the above-described examples.

The training of the first sub-model (S5200) may include training the first sub-model to obtain first output based on the eye image, based on the parameter training data set.

For example, the parameter training set may include the first to seventh labels and the eye image, and the training of the first sub-model (S5200) may include training the first sub-model to obtain first output that includes first to fifth diagnosis assistance information corresponding to the first to fifth labels, respectively, based on the eye image. The training of the first sub-model (S5200) may include updating a parameter of the first sub-model by comparing the first to fifth diagnosis assistance information obtained through the first sub-model based on the eye image, and the first to fifth labels, respectively.

In a more specific example, the parameter training set may include the first label indicating the hematocrit level of the subject, the second label indicating the hemoglobin level of the subject, the third label indicating the age of the subject, the fourth label indicating the gender of the subject, the fifth label indicating the BMI of the subject, the sixth label indicating the degree of risk of the target cerebral cardiovascular disease of the subject, and the seventh label indicating the coronary artery calcium score of the subject, and the training of the first sub-model (S5200) may include training the first sub-model to obtain the first output that includes first diagnosis assistance information indicating the hematocrit level of the subject, second diagnosis assistance information indicating the hemoglobin level of the subject, third diagnosis assistance information indicating the age of the subject, fourth diagnosis assistance information indicating the gender of the subject, and fifth diagnosis assistance information indicating the BMI of the subject, based on the eye image.

The validating of the first output (S5300) may include validating the first output which is obtained by the first sub-model based on the parameter training data set. The validating of the first output (S5300) may include selecting diagnosis assistance information that has accuracy of a predetermined level or higher or lower, from the pieces of diagnosis assistance information included in the first output obtained by the first sub-model, based on the parameter training data set.

For example, the validating of the first output (S5300) may include validating the first output by comparing the first to fifth diagnosis assistance information which are learned by the first sub-model and the first to fifth labels included in the parameter training data set, respectively. The validating of the first output (S5300) may include selecting diagnosis assistance information that has accuracy of the predetermined level or higher or lower, by comparing the first to fifth diagnosis assistance information learned by the first sub-model and the first to fifth labels included in the parameter training data set, respectively.

In a more specific example, the parameter training set may include the first label indicating the hematocrit level of the subject, the second label indicating the hemoglobin level of the subject, the third label indicating the age of the subject, the fourth label indicating the gender of the subject, the fifth label indicating the BMI of the subject, the sixth label indicating the degree of risk of the target cerebral cardiovascular disease of the subject, and the seventh label indicating the coronary artery calcium score of the subject, and the validating of the first output (S5300) may include determining diagnosis assistance information that shows a degree of match of a predetermined level or higher, by comparing the first output including the first diagnosis assistance information indicating the hematocrit level of the subject, the second diagnosis assistance information indicating the hemoglobin level of the subject, the third diagnosis assistance information indicating the age of the subject, the fourth diagnosis assistance information indicating the gender of the subject, and the fifth diagnosis assistance information indicating the BMI of the subject, which are obtained by the first sub-model, with the first to fifth labels.

The training of the second sub-model (S5400) may be performed by using the parameter training data set. The training of the second sub-model (S5400) may include training the second sub-model to obtain second output based on at least part of the diagnosis assistance information, by using the parameter training data set. The training of the second sub-model (S5400) may include training the second sub-model to obtain second output based on the diagnosis assistance information selected by the output validation unit, by using the parameter training data set.

For example, the training of the second sub-model (S5400) may include obtaining, through the second sub-model, sixth and seventh diagnosis assistance information, based on the first to third diagnosis assistance information which are selected as having accuracy of the predetermined level or higher or lower from the first to fifth diagnosis assistance information learned by the first sub-model. The training of the second sub-model (S5400) may include updating the second sub-model by comparing the sixth and seventh diagnosis assistance information with the sixth label and the seventh label which are included in the parameter training data set and correspond to the sixth and seventh diagnosis assistance information.

In a more specific example, the training of the second sub-model (S5400) may include updating a parameter of the second sub-model at least in part, by comparing the sixth diagnosis assistance information indicating the degree of risk of the target cerebral cardiovascular disease of the subject and the seventh diagnosis assistance information indicating the coronary artery calcium score, which are obtained by the second sub-model, with the sixth label and the seventh label included in the parameter training data set, based on the first diagnosis assistance information indicating the hematocrit level of the subject and the second diagnosis assistance information indicating the hemoglobin level of the subject, which are selected by the output validation unit from the first output obtained by the first sub-model.

### 5.3 Parameter Obtaining Diagnosis Assistance

According to an embodiment, there may be provided a method for obtaining at least one parameter through the above-described diagnosis assistance neural network model. According to an embodiment, there may be provided a method for assisting a diagnosis of a target disease based on at least one parameter which is obtained through the above-described diagnosis assistance neural network model.

According to an embodiment, a diagnosis assistance method using a parameter obtaining neural network model may include obtaining a target eye image, and obtaining at least one parameter according to the target eye image.

The target eye image may be an image that is obtained by capturing various shapes of eyes described above. The obtaining of the target eye image may include obtaining one or more eye images, for example, a left-eye image and a right-eye image. The obtaining of the target eye image may include obtaining a medical image that is obtained by capturing a body part other than eyes. The obtaining of the eye image may include obtaining non-visual medical data, for example, body information of a subject, life habit information, or information related to a target disease.

The obtaining of the at least one parameter according to the target eye image may include obtaining the at least one parameter through the above-described parameter obtaining neural network model. The obtaining of the at least one parameter according to the target eye image may include obtaining the at least one parameter and diagnosis assistance information used for a diagnosis of the target disease through the above-described parameter obtaining neural network model.

The obtaining of the at least one parameter according to the target eye image may include obtaining the at least one parameter by using the above-described diagnosis assistance neural network model which includes the plurality of sub-neural network models connected in parallel and obtains a plurality of parameters, the above-described diagnosis assistance neural network model which includes the plurality of sub-neural network models connected in series and obtains a plurality of parameters, the above-described neural network model which includes the common portion and the individual portion and obtains a plurality of parameters, or the above-described neural network model which has the output validation unit.

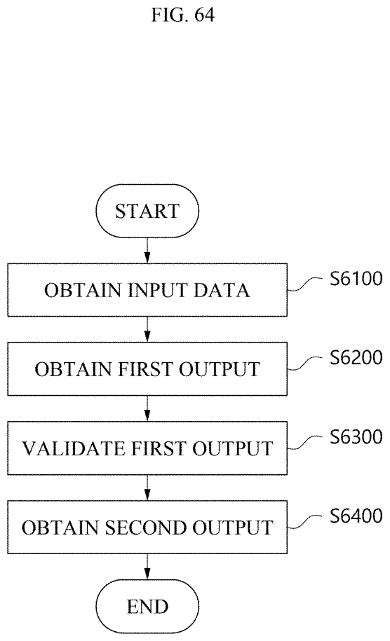

FIG. 64 is a view for describing a diagnosis assistance method according to an embodiment.

FIG. 64 is a view for describing a diagnosis assistance method according to an embodiment. Referring to FIG. 64, the diagnosis assistance method according to an embodiment may include obtaining input data (S6100), obtaining first output (S6200), validating the first output (S6300), and obtaining second output (S6400). Hereinafter, the diagnosis assistance method will be described with reference to the neural network model described in relation with FIG. 62.

The obtaining of the input data (S6100) may include obtaining a target eye image and/or a target medical image obtained by capturing a body part other than eyes and/or non-visual medical data related to a subject. For example, the input data may include body information of the subject, for example, medical data indicating gender, age, height, etc.

The obtaining of the first output (S6200) may include obtaining the first output based on first input included in the input data. The first output may include diagnosis assistance information related to at least one parameter and/or a target disease. For example, the first output may include diagnosis assistance information indicating any one of height, age, or gender of the subject.

The validating of the first output (S6300) may include validating at least part of the diagnosis assistance information included in the first output based on the input data. For example, the validating of the first output (S6300) may include validating the first output by comparing diagnosis assistance information included in the first output and indicating any one of the height, age, or gender of the subject, with real height, age, or gender of the subject included in the input data.

The validating of the first output (S6300) may include selecting at least part of the diagnosis assistance information included in the first output. The validating of the first output (S6300) may include selecting diagnosis assistance information that shows accuracy of a predetermined level or higher (or lower), by comparing at least part of the diagnosis assistance information included in the first output with the input data.

For example, the validating of the first output (S6300) may include selecting, from the diagnosis assistance information included in the first output, first diagnosis assistance information indicating (estimated) age of the subject and second diagnosis assistance information indicating (estimated) gender of the subject, which match real age and real gender of the subject included in the input data by the predetermined level or higher (or lower).

The obtaining of the second output (S6400) may include obtaining the second output based on at least part of the first output. The obtaining of the second output (S6400) may include obtaining the second output based at least in part on second input included in the input data.

For example, the obtaining of the second output (S6400) may obtain the second output based on the first diagnosis assistance information indicating (estimated) age of the subject and the second diagnosis assistance information indicating (estimated) gender of the subject, which match the real age and the real gender of the subject included in the input data by the predetermined level or higher (or lower), from the diagnosis assistance information included in the first output.

The obtaining of the second output (S6400) may include obtaining the second output based on the diagnosis assistance information included in the first output and/or the eye image, the medical image other than the eye image or the non-visual medical data of the subject which is included in the input data.

The second output may include diagnosis assistance information indicating (estimated) medical data or a parameter related to the subject, or diagnosis assistance information related to the target disease of the subject.

According to an embodiment, the diagnosis assistance method may further include obtaining diagnosis assistance information (for example, secondary information) regarding the target disease based on the obtained parameter.

The diagnosis assistance method may obtain at least one parameter through the diagnosis assistance neural network model, and may obtain secondary information for the diagnosis of the target disease, for example, secondary diagnosis assistance information indicating the presence of the target disease of the subject, a degree of risk of the target disease of the subject, and a numerical prediction value related to the target disease of the subject.

In a specific example, the diagnosis assistance method may include obtaining diagnosis assistance information indicating a hematocrit level of the subject through the above-described diagnosis assistance neural network model. In addition, the diagnosis assistance method may include obtaining the secondary diagnosis assistance information indicating the presence of a target cerebral cardiovascular disease of the subject, based on the diagnosis assistance information indicating the hematocrit level of the subject, which is obtained through the diagnosis assistance neural network model.

For example, the diagnosis assistance method may include, when the secondary diagnosis assistance information obtained through the diagnosis assistance neural network model indicates that the hematocrit level of the subject is lower than first value, obtaining secondary diagnosis assistance information indicating that the risk of anemia, a kidney disease, a blood loss, uremide of the subject is high. For example, the diagnosis assistance method may include, when the secondary diagnosis assistance information obtained through the diagnosis assistance neural network model indicates that the hematocrit level of the subject is higher than the first value, obtaining secondary diagnosis assistance information indicating that a probability of the subject having jaundice, a cardiac disorder, polycythaemia, hypoxia, blood doping, or dehydration is high.

According to an embodiment, the diagnosis assistance method may include determining whether a parameter indicated by the obtained diagnosis assistance information is included within a reference range. The diagnosis assistance method may include obtaining diagnosis assistance information indicating that a target parameter is out of the reference range, and obtaining secondary diagnosis assistance information corresponding to the obtained diagnosis assistance information.

For example, the diagnosis assistance method may include obtaining diagnosis assistance information indicating a hematocrit level of the subject through the diagnosis assistance neural network model, and determining whether a hematocrit level of the subject is included in a pre-defined numerical range, based on the obtained diagnosis assistance information. The diagnosis assistance method may include, when the hematocrit level of the subject obtained through the diagnosis assistance neural network model is out of the pre-defined numerical range, obtaining secondary diagnosis assistance information according to the obtained hematocrit level of the subject.

For example, the diagnosis assistance method may include: when the hematocrit level of the subject obtained through the diagnosis assistance neural network model exceeds the pre-defined numerical range, obtaining secondary diagnosis assistance information indicating that a probability of the subject having jaundice, a cardiac disorder, polycythaemia, hypoxia, blood doping, or dehydration is high; when the hematocrit level of the subject obtained through the diagnosis assistance neural network model does not reach the pre-defined numerical range, obtaining secondary diagnosis assistance information indicating that a probability of the subject having jaundice, a cardiac disorder, polycythaemia, hypoxia, blood doping, or dehydration is high; and obtaining secondary diagnosis assistance information indicating that the risk of anemia, a kidney disease, a blood loss, uremide of the subject is high.

The pre-defined range of the hematocrit level may be 40 to 43. The pre-defined range of the hematocrit level may be determined differently according to the gender of the subject. For example, the diagnosis assistance method may obtain diagnosis assistance information indicating a hematocrit level of the subject through the diagnosis assistance neural network model, and may determine whether the hematocrit level of the subject is included with the pre-defined numerical range, based on the obtained diagnosis assistance information, and the pre-defined numerical range may be first numerical range if the gender of the subject is female gender, and may be second numerical range if the gender of the subject is male gender. The first numerical range may be 38 to 42% and the second numerical range may be 42 to 42%.

The diagnosis assistance method may further include obtaining the gender of the subject. The obtaining of the gender of the subject may include receiving an input of the gender of the subject from the user and/or obtaining the gender of the subject based on the eye image of the subject by using the diagnosis assistance neural network model.

The diagnosis assistance method may include determining whether the hematocrit level of the subject obtained through the neural network model is included within a numerical range that is determined according to whether the subject has other diseases. For example, the diagnosis assistance method may include obtaining object information indicating that the subject is a dialysis subject, and determining whether the hematocrit level of the subject obtained through the neural network model is included in third numerical range which is lower than the above-described first numerical range or second numerical range, for example, 33-36%.

The above-described embodiment may be similarly applied to other parameters than the hematocrit level. When the parameter is changed, the pre-defined numerical range and/or the secondary diagnosis assistance information may be changed.

For example, when the parameter obtained by the diagnosis assistance neural network model is a hemoglobin level, the pre-defined numerical range may be 12 to 17 (g/dL). When the parameter obtained by the diagnosis assistance neural network model is a hemoglobin level, the pre-defined numerical range may be 13 to 17 (g/dL) if the subject is a male, and may be 12 to 15 (g/dL) if the subject is a female.

In addition, for example, when the parameter obtained by the diagnosis assistance neural network model is a red blood cell count, the pre-defined numerical range may be 3.8 to 5.6 (10\hat{ }6/μl). When the parameter obtained by the diagnosis assistance neural network model is the red blood cell count, the pre-defined range may be 4.2 to 5.6 (10\hat{ }6/μl) if the subject is a male, and may be 3.8 to 5.1 (10\hat{ }6/μl) if the subject is a female.

In addition, for example, when the parameter obtained by the diagnosis assistance neural network model is a creatinine level, the pre-defined numerical range may be 0.50 to 1.4 (mg/dL).

The diagnosis assistance neural network model according to an embodiment may be provided to classify the target eye image according to the reference range of the parameter described above. For example, the diagnosis assistance neural network model may be provided to obtain first diagnosis assistance information indicating a parameter value obtained based on the eye image, and/or second diagnosis assistance information indicating whether the parameter obtained based on the eye image is smaller than a normal numerical range, within the normal numerical range, or lager than the normal numerical range.

According to an embodiment, the diagnosis assistance method may further include obtaining secondary diagnosis assistance information indicating measures required for the subject based on the obtained parameter diagnosis assistance information.

For example, the diagnosis assistance method may further include obtaining diagnosis assistance information indicating a hematocrit level of the subject by using the diagnosis assistance neural network model, and, when the obtained diagnosis assistance information indicates that the hematocrit level of the subject is out of a pre-defined numerical range, obtaining secondary diagnosis assistance information indicating that administration of a medicine for adjusting the hematocrit level, such as anticoagulant, etc., is recommended to the subject.

In addition, for example, the diagnosis assistance method may further include obtaining diagnosis assistance information indicating a creatinine level of the subject by using the diagnosis assistance neural network model, and, when the obtained diagnosis assistance information indicates that the creatinine level of the subject is out of a pre-defined numerical range, obtaining secondary diagnosis assistance information indicating that administration of a medicine for adjusting the creatinine level, such as steroid, hypotensor, iron preparations, a phosphate binder, a diuretic, antithrombotic, etc., is recommended to the subject.

The diagnosis assistance method according to an embodiment may include obtaining diagnosis assistance information indicating a value (or range) of a parameter through the diagnosis assistance neural network model at first time, and at second time after measures for responding thereto are taken, re-obtaining the diagnosis assistance information indicating the parameter value.

The diagnosis assistance method according to an embodiment may include obtaining diagnosis assistance information indicating a value (or range) of a parameter through the diagnosis assistance neural network model at the first time, and, when the diagnosis assistance information indicates that the value of the parameter is out of a normal numerical range, re-obtaining the diagnosis assistance information indicating the value of the parameter at the second time after measures for normalizing the corresponding parameter value, such as drug prescription, are taken.

The diagnosis assistance method according to an embodiment may include obtaining first diagnosis assistance information indicating a value (or range) of a parameter through the diagnosis assistance neural network model based on an eye image obtained at the first time, and obtaining second diagnosis assistance information indicating a value of a parameter through the diagnosis assistance neural network model based on an eye image obtained at the second time after measure for responding thereto are taken. The diagnosis assistance method may further include obtaining third diagnosis assistance information indicating a change of a corresponding parameter regarding the subject and/or necessity for measures in relation thereto, based on the first diagnosis assistance method and the second diagnosis assistance method.

### 6. Domain Adaptation

According to an embodiment of the invention described in the present specification, there may be provided a method for changing a format (or domain) of a medical image such as an eye image, etc. The changing of the format of the image, which will be described below, may be performed by an information processing device, a control unit of the information processing device or a server device. There may be provided a neural network model trained to change a format of a medical image. The changing of the format of the medical image may be performed by a neural network model which is stored in an information processing device, a control unit of the information processing device, or a server device.

The changing of the image format may be performed by obtaining a target image, determining a format of the target image, and changing the format of the target image. The format of the image may refer to a format that is determined according to a device which captures an image. The format of the image may refer to a type of medical image.

According to an embodiment, there may be provided a method for converting a medical image of first format into a medical image of second format.

The medical image of the first format and the medical image of the second format may refer to different kinds of medical images regarding the same body part. In a specific example, the medical image of the first format may be a fundus image, and the medical image of the second format may be an OCT image.

Alternatively, the medical image of the first format and the medical image of the second format may be images that are obtained by capturing the same kind of medical image by different kinds of devices. In a specific example, the medical image of the first format may be a fundus image that is obtained by capturing by first type device, and the medical image of the second format may be a fundus image that is obtained by capturing by second type device.

According to an embodiment, there may be provided a training method of a conversion neural network model which converts first format image into second format image. The training method of the conversion neural network model may include obtaining image conversion training data and updating a parameter of the conversion neural network model by using the same.

The image conversion training data may include first format image and second format image. The image conversion training data may include unit training data including first format image and second format image corresponding to the first format image.

The training of the conversion neural network model may include obtaining an image that is converted through the conversion neural network model based on the first format image, comparing the second format image corresponding to the first format image and included in the image conversion training data with the converted image, and updating the parameter of the conversion neural network model based on a result of comparing.

For example, the image conversion training data may include first image which is captured by first type fundus camera device, and second image which corresponds to the first image (is obtained from the same fundus as the first image) and is captured by second type fundus camera device.

In addition, for example, the image conversion training data may include first image which is an OCT image obtained through an OCT device, and second image which is a fundus image corresponding to the first image (obtained from the same eye as the first image) and captured by a fundus camera device.

In this case, the training of the conversion image model may include updating the parameter of the conversion neural network model based on a difference between the conversion image obtained with the first image as an input image, and the second image.

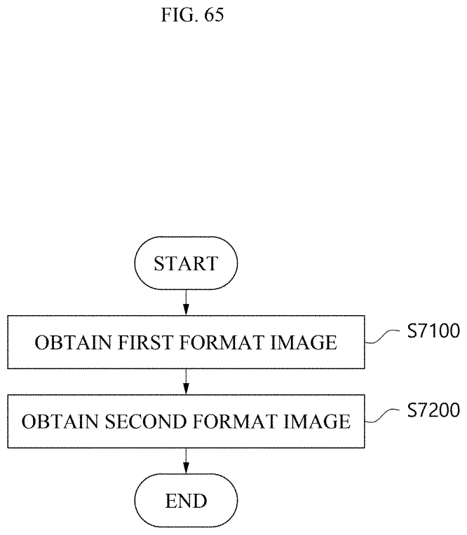

FIG. 65 is a view for describing a method for changing an image format according to an embodiment. Referring to FIG. 65, the method for changing the image format according to an embodiment may include obtaining first format image (S7100) and obtaining second format image (S7200).

The obtaining of the first format image (S7100) may include obtaining an image and determining a format of a target image. The determining of the format of the target image may include determining the format of the target image based on a tag or metadata included in target image data. For example, the obtaining of the first format image (S7100) may include obtaining fundus image data and obtaining fundus image capturing device information included in the fundus image data.

The obtaining of the second format image (S7200) may include obtaining the second format image through the conversion neural network model for converting an image format with the first format image as input data.

According to an embodiment, the conversion neural network model may be trained and provided to convert first format fundus image captured by first type fundus camera device into second format fundus image captured by second type fundus camera device. Alternatively, the conversion neural network model may be trained and provided to convert first format image, which is an OCT image obtained through an OCT device, into second format image which is a fundus image captured by a fundus camera device.

According to an embodiment, the conversion neural network model may be a style transfer network model. For example, the conversion neural network model may be provided to convert first format image into second format image by using a generative adversarial network (GAN) model.

According to an embodiment, the conversion neural network model may obtain images of a plurality of formats based on one image. In this case, the conversion neural network model may use the above-described plurality of information obtaining model types.

### 7. Eye Image Classification

According to an embodiment, there may be provided a method for classifying an eye image. Specifically, there may be provided a method for classifying an eye image into a left eye image obtained by capturing the left eye of a subject and a right eye image obtained by capturing the right eye of the subject.

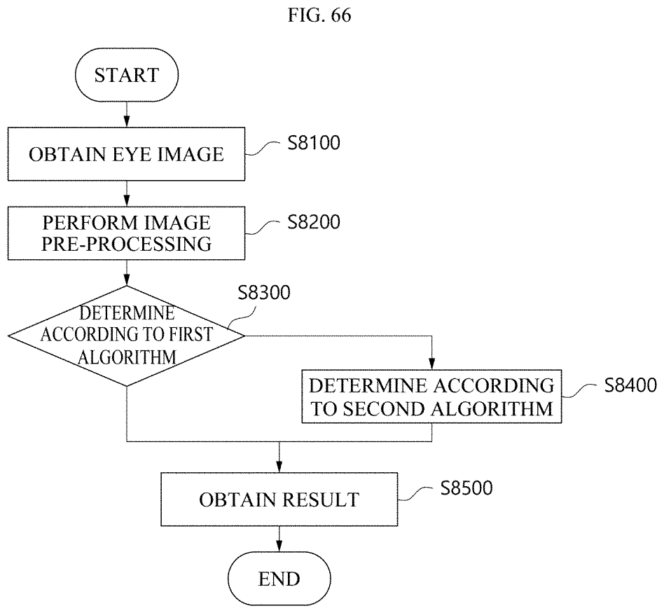

FIG. 66 is a view for describing a method for classifying an eye image according to an embodiment. Referring to FIG. 65, the method for classifying the eye image may include obtaining an eye image (S8100), performing pre-processing with respect to the eye image (S8200), performing first determination with respect to the pre-processed image based on first algorithm (S8300), determining second determination based on second algorithm (S8400), and obtaining a result of determining (S8500).

The obtaining of the eye image (S8100) may include obtaining any one of images obtained by capturing eyes of the subject, for example, an OCT image, a fundus image, an extraocular image, or an iris image.

The performing of the pre-processing with respect to the eye image (S8200) may include performing pre-processing for making it easy to identify elements included in the target eye image with respect to the target eye image. For example, the performing of the pre-processing with respect to the eye image may include performing pre-processing of emphasizing a blood vessel included in the target eye image or emphasizing a specific color included in the target eye image. Alternatively, the performing of the pre-processing with respect to the eye image may include extracting a specific element (for example, an optic disc, a blood vessel or macula) included in the target eye image.

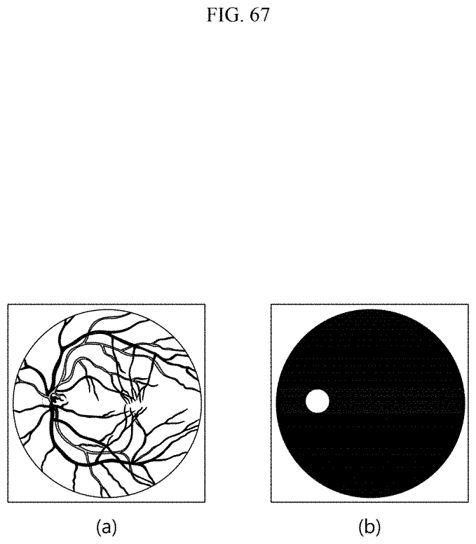

FIG. 67 is a view for describing image pre-processing according to an embodiment. Referring to FIG. 67, the method for classifying the eye image according to an embodiment may include extracting an area where an optic disc is positioned from a fundus image (a). For example, the method for classifying the eye image according to an embodiment may include extracting a position of the optic disc included in the fundus image, and obtaining a binarized image as shown in view (b) of FIG. 67.

Referring back to FIG. 66, the performing of the first determination with respect to the pre-processed image based on the first algorithm may include determining whether the target eye image satisfies a predetermined criterion based on the pre-processed image. The performing of the first determination (S8300) may include determining whether the element extracted from the target eye image is positioned within a pre-defined area of the eye image. Alternatively, the performing of the first determination (S8300) may include determining whether the element extracted from the target eye image exceeds a predetermined ratio of the entire area of the eye image.

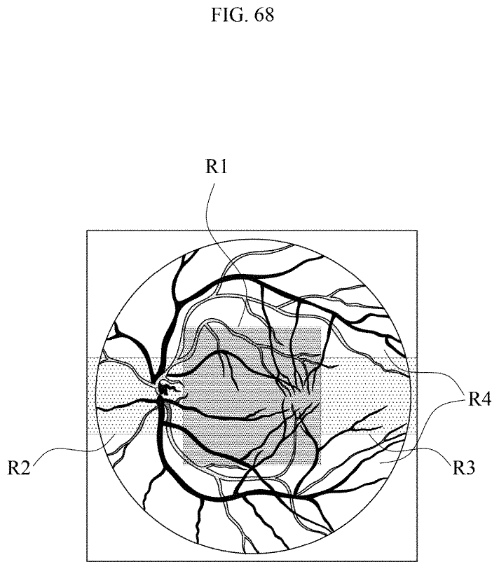

FIG. 68 is a view for describing an embodiment for determining whether an eye image satisfies a predetermined criterion. Referring to FIG. 68, the performing of the first determination based on the first algorithm (S8300) may include determining an area that overlaps an area of the optic disc obtained from the pre-processed image among first area R1, second area R2, third area R3, and fourth area R4 of the eye image. The determining of the area that overlaps the area of the optic disc may include determining an area that has the largest portion overlapping the area of the optic disc among the first to fourth areas.

The performing of the first determination based on the first algorithm (S8300) may include, when the area of the optic disc overlaps the first area R1, determining that the target eye image is a left eye image.

The performing of the first determination based on the first algorithm (S8300) may include, when the area of the optic disc overlaps the second area R2, determining that the target eye image is a right eye image.

The performing of the first determination based on the first algorithm (S8300) may include, when the area of the optic disc overlaps the third area R3, determining that the second determination based on the second algorithm is required for the target eye image.

The performing of the first determination based on the first algorithm (S8300) may include, when the area of the optic disc overlaps the fourth area R4, determining that the target eye image is other images.

The performing of the second determination based on the second algorithm (S8400) may include classifying the target eye image into a right eye image or a left eye image through a classification neural network model which is trained to classify an eye image into a left eye image or a right eye image.

The classification neural network model may include at least one convolution neural network layer and/or at least one fully connected layer. The classification neural network model may be provided as a machine learning model of a classifier type, which extracts a feature set based on an eye image and classifies the eye image into a both-eye image or a right eye image based on the extracted feature set.

The classification neural network model may be trained and provided to obtain both-eye information indicating whether an input eye image is a left eye image or a right eye image. The classification neural network model may be trained based on a both-eye training data set including an eye image and a both-eye label matching the eye image and indicating whether a corresponding eye image is a both-eye image or a right-eye image.

The obtaining of the result of determining (S8500) may include obtaining a result of the first determination based on the first algorithm or the second determination based on the second algorithm.

For example, the obtaining of the result of determining (S8500) may include, when it is determined that the area of the optic disc is positioned in the second area as a result of the first determination based on the first algorithm, obtaining a result of determination indicating that the target eye image is the left eye image, and, when the area of the optic disc is positioned in the third area as a result of the first determination based on the first algorithm, obtaining a result of determining indicating that the target eye image is the right eye image.

In addition, for example, the obtaining of the result of determining (S8500) may include, when it is determined that the area of the optic disc is positioned in the first area as a result of the first determination based on the first algorithm, and a result of classifying indicating that the target eye image is the right-eye image is obtained as a result of the second determination based on the classification neural network model, obtaining a result of determining indicating that the target eye image is the right eye image. In addition, the obtaining of the result of determining (S8500) may include, when it is determined that the area of the optic disc is positioned in the first area as ae result of the first determination based on the first algorithm, and a result of classifying indicating that the target eye image is the left eye image is obtained as a result of the second determination based on the classification neural network model, obtaining a result of determining indicating that the target eye image is the left eye image.

In addition, for example, the obtaining of the result of determining (S8500) may include, when it is determined that the area of the optic disc is positioned in the fourth area as a result of the first determination based on the first algorithm, obtaining a result of failing-to-determine indicating that it is impossible to determine the target eye image as left/right eye images. Alternatively, the obtaining of the result of determining (S8500) may include, when the area of the optic disc is positioned in the fourth area as a result of the first determination based on the first algorithm, obtaining both-eye information of the target eye image through the classification neural network model.

The obtaining of the result of determining (S8500) may include, when the area of the optic disc is the first area, obtaining both-eye information of the target eye image through first classification neural network model, and, when the area of the optic disc is the fourth area, obtaining both-eye information of the target eye image through second neural network model which is different from the first classification neural network model at least in part.

According to an embodiment, there may be provided a method for obtaining an eye image data set based on the above-described eye image classification method. For example, the method for obtaining the data set may include classifying an input eye image based on the above-described eye image classification method, and labeling the input eye image with a result of classifying. The method for obtaining the data set may include obtaining an eye image data set including an eye image and a classification label assigned to the eye image, by using the above-described classification method.

In the above-described embodiments, the case in which a method for determining quality of an eye image or determining suitability is applied to establishment of a database, training of a neural network model, and operating of the neural network model has been described, but the contents of the invention disclosed in the present specification are not limited thereto. Even in the case of an image other than an eye image, a defect may occur in a predetermined area of the image, and, when predetermined information is obtained based on the image, the contents of the invention disclosed in the present specification may be analogically applied.

Although the embodiments have been described with reference to specified embodiments and drawings as described above, various modifications and changes may be made by a person skilled in the art. For example, even if the above-described technologies are performed in an order different from the methods described above, and/or components described above such as systems, structures, device, circuits, etc. are coupled or combined in forms different from the methods described above, or are replaced or substituted with other components or equivalents, appropriate results may be achieved.

Accordingly, other implementations, other embodiments and equivalents to the claims belong to the scope of the claims described below.

What is claimed is:

1. A diagnosis assistance apparatus which uses a neural network model comprising at least one neural network layer and is configured to obtain diagnosis assistance information based on an eye image, the diagnosis assistance apparatus comprising:
an eye image obtaining unit configured to obtain a target eye image which is obtained from eyes of a subject; and
a processing unit configured to use a neural network model trained to obtain diagnosis assistance information based on the eye image, and obtain the diagnosis assistance information based on the target eye image,
wherein the neural network model comprises: first diagnosis assistance neural network model configured to obtain first diagnosis assistance information based on the target eye image; and second diagnosis assistance neural network model configured to obtain second diagnosis assistance information which is different from the first diagnosis assistance information, based on the target eye image,
wherein the first diagnosis assistance neural network model comprises: first common portion configured to obtain first feature set based on the target eye image; and first individual portion configured to obtain the first diagnosis assistance information based on the first feature set,
wherein the second diagnosis assistance neural network model comprises: the first common portion configured to obtain the first feature set based on the target eye image; and second individual portion configured to obtain the second diagnosis assistance information based on the first feature set,
wherein the first individual portion is trained based on first training data, and the first individual portion is trained based on second training data which is different from the first training data at least in part.

2. The diagnosis assistance apparatus of claim 1, wherein the first feature set comprises a plurality of feature values which are associated with the first diagnosis assistance information and the second diagnosis assistance information,
wherein the first individual portion is configured to obtain the first diagnosis assistance information based on at least one feature value included in the first feature set, and
wherein the second individual portion is configured to obtain the second diagnosis assistance information based on at least one feature value included in the first feature set.

3. The diagnosis assistance apparatus of claim 2, wherein the first diagnosis assistance information comprises first information and second information, and
wherein the first individual portion comprises:
second common portion configured to obtain second feature set which comprises a plurality of feature values associated with the first information and the second information, based at least in part on the first feature set;
first sub-portion configured to obtain the first information based at least in part on the second feature set; and
second sub-portion configured to obtain the second information based at least in part on the second feature set.

4. The diagnosis assistance apparatus of claim 1, wherein the first diagnosis assistance information comprises at least one piece of diagnosis assistance information related to an eye disease, and the second diagnosis assistance information comprises at least one piece of diagnosis assistance information related to a cerebral cardiovascular disease.

5. The diagnosis assistance apparatus of claim 1, wherein the first diagnosis assistance information comprises at least one piece of diagnosis assistance information related first eye disease, and the second diagnosis assistance information comprises at least one piece of diagnosis assistance information related to second eye disease which is different from the first eye disease.

6. The diagnosis assistance apparatus of claim 1, wherein the first diagnosis assistance information comprises diagnosis assistance information related to glaucoma, and the second diagnosis assistance information comprises diagnosis assistance information related to a coronary artery disease.

7. The diagnosis assistance apparatus of claim 1, wherein the processing unit further comprises a pre-processing unit configured to perform pre-processing for emphasizing a blood vessel included in the target eye image and to obtain a blood vessel-emphasized eye image, and
wherein the first common portion is configured to obtain the first feature set based on the blood vessel-emphasized eye image.

8. The diagnosis assistance apparatus of claim 3, wherein the first information and the second information are diagnosis assistance information related to a disease related to first part of a human body, and the second diagnosis assistance information is diagnosis assistance information related to a disease related to second part of the human body, the second part being different from the first part.

9. The diagnosis assistance apparatus of claim 3, wherein the first information is diagnosis assistance information indicating whether the eyes of the subject correspond to glaucoma, and the second information is diagnosis assistance information indicating whether the eyes of the subject correspond to diabetic retinopathy, and
wherein the second diagnosis assistance information is diagnosis assistance information indicating a degree of calcification of a coronary artery of the subject.

10. The diagnosis assistance apparatus of claim 1, wherein the first feature set comprises at least one feature map.

11. The diagnosis assistance apparatus of claim 3, wherein the first feature set comprises at least one feature map, and the second feature set comprises at least one feature value.

12. A method for assisting a diagnosis by using a diagnosis assistance apparatus, the diagnosis assistance apparatus comprising an eye image obtaining unit configured to obtain an eye image, and a processing unit configured to obtain diagnosis assistance information based on the eye image by using a neural network model, the neural network model comprising at least one neural network layer and being trained to obtain the diagnosis assistance information based on the eye image,
wherein the neural network model comprises: first diagnosis assistance neural network model configured to obtain first diagnosis assistance information based on the eye image; and second diagnosis assistance neural network model configured to obtain second diagnosis assistance information based on the eye image,
wherein the first diagnosis assistance neural network model comprises first common portion and first individual portion, and the second diagnosis assistance neural network model comprises the first common portion and second individual portion,
wherein the diagnosis assistance method comprises:
obtaining, by the eye image obtaining unit, a target eye image which is obtained from eyes of a subject;
obtaining, by the processing unit, a first feature set based on the target eye image through the first common portion;
obtaining, by the processing unit, the first diagnosis assistance information based at least in part on the first feature set through the first individual portion; and
obtaining, by the processing unit, the second diagnosis assistance information based at least in part on the first feature set through the second individual portion,
wherein the first individual portion is trained based on first training data, and the second individual portion is trained based on second training data which is different from the first training data at least in part.

13. The diagnosis assistance method of claim 12, wherein the first diagnosis assistance information comprises first information and second information, and the first individual portion comprises second common portion, first sub-portion and second sub-portion,
wherein obtaining the first diagnosis assistance information comprises:
obtaining, by the second common portion, second feature set which is associated with the first information and the second information, based at least in part on the first feature set;
obtaining, by the first sub-portion, the first information based at least in part on the second feature set; and
obtaining, by the second sub-portion, the second information based at least in part on the second feature set.

14. The diagnosis assistance method of claim 13, wherein the first feature set comprises at least one feature map, and the second feature set comprises at least one feature value.

15. The diagnosis assistance method of claim 13, wherein the first information and the second information are diagnosis assistance information related to a disease related to first part of a human body, and the second diagnosis assistance information is diagnosis assistance information related to a disease related to second part of the human body, the second part being different from the first part.

16. The diagnosis assistance method of claim 12, wherein the first diagnosis assistance information comprises at least one piece of diagnosis assistance information related first eye disease, and the second diagnosis assistance information comprises at least one piece of diagnosis assistance information related to second eye disease which is different from the first eye disease.

17. The diagnosis assistance method of claim 12, wherein the first feature set comprises at least one feature map.

18. The diagnosis assistance method of claim 12, wherein the processing unit further comprises a pre-processing unit configured to perform pre-processing for emphasizing a blood vessel included in the target eye image and to obtain a blood vessel-emphasized eye image, and
wherein obtaining the first feature set comprises obtaining the first feature set based on the blood vessel-emphasized eye image through the first common portion.

19. The diagnosis assistance method of claim 12, wherein the first diagnosis assistance information comprises at least one piece of diagnosis assistance information related to an eye disease, and the second diagnosis assistance information comprises at least one piece of diagnosis assistance information related to a cerebral cardiovascular disease.

20. A computer-readable recording medium having a program recorded thereon to perform the method according to claim 12.
